SEQUENCE LISTING (141 sequences)

SEQ ID NO: 1 - protein, 125 aa, Homo sapiens
  sequence of CXCL9
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 2 - protein, 98 aa, Homo sapiens
  sequence of CXCL10
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 3 - protein, 93 aa, Homo sapiens
  sequence of CXCL11
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 4 - protein, 93 aa, Homo sapiens; identical to SEQ ID NO: 3
  sequence of CXCL12

SEQ ID NO: 5 - protein, 109 aa, Homo sapiens
  sequence of CXCL13
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 6 - protein, 368 aa, Homo sapiens
  sequence of CXCR3-1
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 7 - protein, 415 aa, Homo sapiens
  sequence of CXCR3-2
MELRKYGPGRLAGTVIGGAAQSKSQTKSDSITKEFLPGLYTAPSSPFPPSQVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 8 - protein, 372 aa, Homo sapiens
  sequence of CXCR5-1
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 9 - protein, 327 aa, Homo sapiens
  sequence of CXCR5-2
MASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 10 - protein, 107 aa, Homo sapiens
  sequence of CXCL1
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 11 - protein, 107 aa, Homo sapiens
  sequence of CXCL2
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 12 - protein, 107 aa, Homo sapiens
  sequence of CXCL3
MAHATLSAAPSNPRLLRVALLLLLLVAASRRAAGASVVTELRCQCLQTLQGIHLKNIQSVNVRSPGPHCAQTEVIATLKNGKKACLNPASPMVQKIIEKILNKGSTN

SEQ ID NO: 13 - protein, 101 aa, Homo sapiens
  sequence of CXCL4
MSSAAGFCASRPGLLFLGLLLLPLVVAFASAEAEEDGDLQCLCVKTTSQVRPRHITSLEVIKAGPHCPTAQLIATLKNGRKICLDLQAPLYKKIIKKLLES

SEQ ID NO: 14 - protein, 114 aa, Homo sapiens
  sequence of CXCL5
MSLLSSRAARVPGPSSSLCALLVLLLLLTQPGPIASAGPAAAVLRELRCVCLQTTQGVHPKMISNLQVFAIGPQCSKVEVVASLKNGKEICLDPEAPFLKKVIQKILDGGNKEN

SEQ ID NO: 15 - protein, 114 aa, Homo sapiens
  sequence of CXCL6
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLKKVIQKILDSGNKKN

SEQ ID NO: 16 - protein, 128 aa, Homo sapiens
  sequence of CXCL7
MSLRLDTTPSCNSARPLHALQVLLLLSLLLTALASSTKGQTKRNLAKGKEESLDSDLYAELRCMCIKTTSGIHPKNIQSLEVIGKGTHCNQVEVIATLKDGRKICLDPDAPRIKKIVQKKLAGDESAD

SEQ ID NO: 17 - protein, 99 aa, Homo sapiens
  sequence of CXCL8/IL-8
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 18 - protein, 273 aa, Homo sapiens
  sequence of CXCL16
MSGSQSEVAPSPQSPRSPEMGRDLRPGSRVLLLLLLLLLVYLTQPGNGNEGSVTGSCYCGKRISSDSPPSVQFMNRLRKHLRAYHRCLYYTRFQLLSWSVCGGNKDPWVQELMSCLDLKECGHAYSGIVAHQKHLLPTSPPISQASEGASSDIHTPAQMLLSTLQSTQRPTLPVGSLSSDKELTRPNETTIHTAGHSLAAGPEAGENQKQPEKNAGPTARTSATVPVLCLLAIIFILTAALSYVLCKRRRGQSPQSSPDLPVHYIPVAPDSNT

SEQ ID NO: 19 - protein, 350 aa, Homo sapiens
  sequence of CXCR1
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 20 - protein, 360 aa, Homo sapiens
  sequence of CXCR2
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 21 - protein, 356 aa, Homo sapiens
  sequence of CXCR4a
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 22 - protein, 352 aa, Homo sapiens
  sequence of CXCR4b
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 23 - protein, 342 aa, Homo sapiens
  sequence of CXCR6
MAEHDYHEDYGFSSFNDSSQEEHQDFLQFSKVFLPCMYLVVFVCGLVGNSLVLVISIFYHKLQSLTDVFLVNLPLADLVFVCTLPFWAYAGIHEWVFGQVMCKSLLGIYTINFYTSMLILTCITVDRFIVVVKATKAYNQQAKRMTWGKVTSLLIWVISLLVSLPQIIYGNVFNLDKLICGYHDEAISTVVLATQMTLGFFLPLLTMIVCYSVIIKTLLHAGGFQKHRSLKIIFLVMAVFLLTQMPFNLMKFIRSTHWEYYAMTSFHYTIMVTEAIAYLRACLNPVLYAFVSLKFRKNFWKLVKDIGCLPYLGVSHQWKSSEDNSKTFSASHNVEATSMFQL

SEQ ID NO: 24 - protein, 96 aa, Homo sapiens
  sequence of CCL1
MQIITTALVCLLLAGMWPEDVDSKSMQVPFSRCCFSFAEQEIPLRAILCYRNTSSICSNEGLIFKLKRGKEACALDTVGWVQRHRKMLRHCPSKRK

SEQ ID NO: 25 - protein, 99 aa, Homo sapiens
  sequence of CCL2
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 26 - protein, 92 aa, Homo sapiens
  sequence of CCL3
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 27 - protein, 92 aa, Homo sapiens
  sequence of CCL4-1
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 28 - protein, 92 aa, Homo sapiens
  sequence of CCL4L1
MKLCVTVLSLLVLVAAFCSLALSAPMGSDPPTACCFSYTARKLPRNFVVDYYETSSLCSQPAVVFQTKRGKQVCADPSESWVQEYVYDLELN

SEQ ID NO: 29 - protein, 91 aa, Homo sapiens
  sequence of CCL5
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 30 - protein, 99 aa, Homo sapiens
  sequence of CCL7
MKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 31 - protein, 99 aa, Homo sapiens
  sequence of CCL8
MKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

SEQ ID NO: 32 - protein, 97 aa, Homo sapiens
  sequence of CCL11
MKVSAALLWLLLIAAAFSPQGLAGPASVPTTCCFNLANRKIPLQRLESYRRITSGKCPQKAVIFKTKLAKDICADPKKKWVQDSMKYLDQKSPTPKP

SEQ ID NO: 33 - protein, 98 aa, Homo sapiens
  sequence of CCL13
MKVSAVLLCLLLMTAAFNPQGLAQPDALNVPSTCCFTFSSKKISLQRLKSYVITTSRCPQKAVIFRTKLGKEICADPKEKWVQNYMKHLGRKAHTLKT

SEQ ID NO: 34 - protein, 93 aa, Homo sapiens
  sequence of CCL14-1
MKISVAAIPFFLLITIALGTKTESSSRGPYHPSECCFTYTTYKIPRQRIMDYYETNSQCSKPGIVFITKRGHSVCTNPSDKWVQDYIKDMKEN

SEQ ID NO: 35 - protein, 109 aa, Homo sapiens
  sequence of CCL14-2
MKISVAAIPFFLLITIALGTKTESSSQTGGKPKVVKIQLKLVGGPYHPSECCFTYTTYKIPRQRIMDYYETNSQCSKPGIVFITKRGHSVCTNPSDKWVQDYIKDMKEN

SEQ ID NO: 36 - protein, 113 aa, Homo sapiens
  sequence of CCL15
MKVSVAALSCLMLVAVLGSQAQFINDAETELMMSKLPLENPVVLNSFHFAADCCTSYISQSIPCSLMKSYFETSSECSKPGVIFLTKKGRQVCAKPSGPGVQDCMKKLKPYSI

SEQ ID NO: 37 - protein, 120 aa, Homo sapiens
  sequence of CCL16
MKVSEAALSLLVLILIITSASRSQPKVPEWVNTPSTCCLKYYEKVLPRRLVVGYRKALNCHLPAIIFVTKRNREVCTNPNDDWVQEYIKDPNLPLLPTRNLSTVKIITAKNGQPQLLNSQ

SEQ ID NO: 38 - protein, 94 aa, Homo sapiens
  sequence of CCL17
MAPLKMLALVTLLLGASLQHIHAARGTNVGRECCLEYFKGAIPLRKLKTWYQTSEDCSRDAIVFVTVQGRAICSDPNNKRVKNAVKYLQSLERS

SEQ ID NO: 39 - protein, 89 aa, Homo sapiens
  sequence of CCL18
MKGLAAALLVLVCTMALCSCAQVGTNKELCCLVYTSWQIPQKFIVDYSETSPQCPKPGVILLTKRGRQICADPNKKWVQKYISDLKLNA

SEQ ID NO: 40 - protein, 98 aa, Homo sapiens
  sequence of CCL19
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 41 - protein, 96 aa, Homo sapiens
  sequence of CCL20-1
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 42 - protein, 95 aa, Homo sapiens
  sequence of CCL20-2
MCCTKSLLLAALMSVLLLHLCGESEASNFDCCLGYTDRILHPKFIVGFTRQLANEGCDINAIIFHTKKKLSVCANPKQTWVKYIVRLLSKKVKNM

SEQ ID NO: 43 - protein, 93 aa, Homo sapiens
  sequence of CCL22
MDRLQTALLVVLVLLAVALQATEAGPYGANMEDSVCCRDYVRYRLPLRVVKHFYWTSDSCPRPGVVLLTFRDKEICADPRVPWVKMILNKLSQ

SEQ ID NO: 44 - protein, 120 aa, Homo sapiens
  sequence of CCL23-CKbeta8
MKVSVAALSCLMLVTALGSQARVTKDAETEFMMSKLPLENPVLLDRFHATSADCCISYTPRSIPCSLLESYFETNSECSKPGVIFLTKKGRRFCANPSDKQVQVCVRMLKLDTRIKTRKN

SEQ ID NO: 45 - protein, 137 aa, Homo sapiens
  sequence of CCL23-CKbeta8-1
MKVSVAALSCLMLVTALGSQARVTKDAETEFMMSKLPLENPVLLDMLWRRKIGPQMTLSHAAGFHATSADCCISYTPRSIPCSLLESYFETNSECSKPGVIFLTKKGRRFCANPSDKQVQVCVRMLKLDTRIKTRKN

SEQ ID NO: 46 - protein, 119 aa, Homo sapiens
  sequence of CCL24
MAGLMTIVTSLLFLGVCAHHIIPTGSVVIPSPCCMFFVSKRIPENRVVSYQLSSRSTCLKAGVIFTTKKGQQFCGDPKQEWVQRYMKNLDAKQKKASPRARAVAVKGPVQRYPGNQTTC

SEQ ID NO: 47 - protein, 150 aa, Homo sapiens
  sequence of CCL25-1
MNLWLLACLVAGFLGAWAPAVHTQGVFEDCCLAYHYPIGWAVLRRAWTYRIQEVSGSCNLPAAIFYLPKRHRKVCGNPKSREVQRAMKLLDARNKVFAKLHHNTQTFQAGPHAVKKLSSGNSKLSSSKFSNPISSSKRNVSLLISANSGL

SEQ ID NO: 48 - protein, 149 aa, Homo sapiens
  sequence of CCL25-2
MNLWLLACLVAGFLGAWAPAVHTQGVFEDCCLAYHYPIGWAVLRRAWTYRIQEVSGSCNLPAAIFYLPKRHRKVCGNPKSREVQRAMKLLDARNKVFAKLHHNTQTFQGPHAVKKLSSGNSKLSSSKFSNPISSSKRNVSLLISANSGL

SEQ ID NO: 49 - protein, 84 aa, Homo sapiens
  sequence of CCL25-CRA_a
MNLWLLACLVAGFLGAWAPAVHTQGVFEDCCLAYHYPIGWAVLRRAWTYRIQEVSGSCNLPAAIRPSCCKEVEFWKLQVIIIQV

SEQ ID NO: 50 - protein, 94 aa, Homo sapiens
  sequence of CCL26
MMGLSLASAVLLASLLSLHLGTATRGSDISKTCCFQYSHKPLPWTWVRSYEFTSNSCSQRAVIFTTKRGKKVCTHPRKKWVQKYISLLKTPKQL

SEQ ID NO: 51 - protein, 112 aa, Homo sapiens
  sequence of CCL27
MKGPPTFCSLLLLSLLLSPDPTAAFLLPPSTACCTQLYRKPLSDKLLRKVIQVELQEADGDCHLQAFVLHLAQRSICIHPQNPSLSQWFEHQERKLHGTLPKLNFGMLRKMG

SEQ ID NO: 52 - protein, 374 aa, Homo sapiens
  sequence of CCR2-A
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 53 - protein, 360 aa, Homo sapiens
  sequence of CCR2-B
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 54 - protein, 355 aa, Homo sapiens
  sequence of CCR3-1
MTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 55 - protein, 376 aa, Homo sapiens
  sequence of CCR3-2
MPFGIRMLLRAHKPGSSRRSEMTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 56 - protein, 373 aa, Homo sapiens
  sequence of CCR3-3
MPFGIRMLLRAHKPGRSEMTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 57 - protein, 360 aa, Homo sapiens
  sequence of CCR4
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 58 - protein, 352 aa, Homo sapiens [sequence omitted]

SEQ ID NO: 59 - protein, 374 aa, Homo sapiens
  sequence of CCR6
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 60 - protein, 355 aa, Homo sapiens
  sequence of CCR8
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 61 - protein, 369 aa, Homo sapiens
  sequence of CCR9A
MTPTDFTSPIPNMADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 62 - protein, 357 aa, Homo sapiens
  sequence of CCR9B
MADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 63 - protein, 362 aa, Homo sapiens
  sequence of CCR10
MGTEATEQVSWGHYSGDEEDAYSAEPLPELCYKADVQAFSRAFQPSVSLTVAALGLAGNGLVLATHLAARRAARSPTSAHLLQLALADLLLALTLPFAAAGALQGWSLGSATCRTISGLYSASFHAGFLFLACISADRYVAIARALPAGPRPSTPGRAHLVSVIVWLLSLLLALPALLFSQDGQREGQRRCRLIFPEGLTQTVKGASAVAQVALGFALPLGVMVACYALLGRTLLAARGPERRRALRVVVALVAAFVVLQLPYSLALLLDTADLLAARERSCPASKRKDVALLVTSGLALARCGLNPVLYAFLGLRFRQDLRRLLRGGSCPSGPQPRRGCPRRPRLSSCSAPTETHSLSWDN

SEQ ID NO: 64 - protein, 350 aa, Homo sapiens
  sequence of CCR11
MALEQNQSTDYYYEENEMNGTYDYSQYELICIKEDVREFAKVFLPVFLTIVFVIGLAGNSMVVAIYAYYKKQRTKTDVYILNLAVADLLLLFTLPFWAVNAVHGWVLGKIMCKITSALYTLNFVSGMQFLACISIDRYVAVTKVPSQSGVGKPCWIICFCVWMAAILLSIPQLVFYTVNDNARCIPIFPRYLGTSMKALIQMLEICIGFVVPFLIMGVCYFITARTLMKMPNIKISRPLKVLLTVVIVFIVTQLPYNIVKFCRAIDIIYSLITSCNMSKRMDIAIQVTESIALFHSCLNPILYVFMGASFKNYVMKVAKKYGSWRRQRQSVEEFPFDSEGPTEPTSTFSI

SEQ ID NO: 65 - protein, 344 aa, Homo sapiens
  sequence of CCRL2-1
MANYTLAPEDEYDVLIEGELESDEAEQCDKYDAQALSAQLVPSLCSAVFVIGVLDNLLVVLILVKYKGLKRVENIYLLNLAVSNLCFLLTLPFWAHAGGDPMCKILIGLYFVGLYSETFFNCLLTVQRYLVFLHKGNFFSARRRVPCGIITSVLAWVTAILATLPEFVVYKPQMEDQKYKCAFSRTPFLPADETFWKHFLTLKMNISVLVLPLFIFTFLYVQMRKTLRFREQRYSLFKLVFAIMVVFLLMWAPYNIAFFLSTFKEHFSLSDCKSSYNLDKSVHITKLIATTHCCINPLLYAFLDGTFSKYLCRCFHLRSNTPLQPRGQSAQGTSREEPDHSTEV

SEQ ID NO: 66 - protein, 356 aa, Homo sapiens
  sequence of CCRL2-2
MIYTRFLKGSLKMANYTLAPEDEYDVLIEGELESDEAEQCDKYDAQALSAQLVPSLCSAVFVIGVLDNLLVVLILVKYKGLKRVENIYLLNLAVSNLCFLLTLPFWAHAGGDPMCKILIGLYFVGLYSETFFNCLLTVQRYLVFLHKGNFFSARRRVPCGIITSVLAWVTAILATLPEFVVYKPQMEDQKYKCAFSRTPFLPADETFWKHFLTLKMNISVLVLPLFIFTFLYVQMRKTLRFREQRYSLFKLVFAIMVVFLLMWAPYNIAFFLSTFKEHFSLSDCKSSYNLDKSVHITKLIATTHCCINPLLYAFLDGTFSKYLCRCFHLRSNTPLQPRGQSAQGTSREEPDHSTEV

SEQ ID NO: 67 - protein, 114 aa, Homo sapiens
  sequence of XCL1
MRLLILALLGICSLTAYIVEGVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 68 - protein, 333 aa, Homo sapiens
  sequence of XCR1
MESSGNPESTTFFYYDLQSQPCENQAWVFATLATTVLYCLVFLLSLVGNSLVLWVLVKYESLESLTNIFILNLCLSDLVFACLLPVWISPYHWGWVLGDFLCKLLNMIFSISLYSSIFFLTIMTIHRYLSVVSPLSTLRVPTLRCRVLVTMAVWVASILSSILDTIFHKVLSSGCDYSELTWYLTSVYQHNLFFLLSLGIILFCYVEILRTLFRSRSKRRHRTVKLIFAIVVAYFLSWGPYNFTLFLQTLFRTQIIRSCEAKQQLEYALLICRNLAFSHCCFNPVLYVFVGVKFRTHLKHVLRQFWFCRLQAPSPASIPHSPGAFAYEGASFY

SEQ ID NO: 69 - protein, 387 aa, Homo sapiens
  sequence of CX3CR1a
MREPLEAFKLADLDFRKSSLASGWRMASGAFTMDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 70 - protein, 355 aa, Homo sapiens
  sequence of CX3CR1b
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 71 - protein, 397 aa, Homo sapiens
MAPISLSWLLRLATFCHLTVLLAGQHHGVTKCNITCSKMTSKIPVALLIHYQQNQASCGKRAIILETRQHRLFCADPKEQWVKDAMQHLDRQAAALTRNGGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQAVGLLAFLGLLFCLGVAMFTYQSLQGCPRKMAGEMAEGLRYIPRSCGSNSYVLVPV

SEQ ID NO: 72 - DNA, 2545 bp, Homo sapiens
  sequence of CXCL9
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 73 - DNA, 1227 bp, Homo sapiens
  sequence of CXCL10
ctttgcagataaatatggcacactagccccacgttttctgagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaagaaaaaaaaaaaa

SEQ ID NO: 74 - DNA, 1610 bp, Homo sapiens
  sequence of CXCL11
agagaacaaaacagaaactcttggaagcaggaaaggtgcatgactcaaagagggaaattcctgtgccataaaaggattgctggtgtataaaatgctctatatatgccaattatcaatttcctttcatgttcagcatttctactccttccaagaagagcagcaaagctgaagtagcagcagcagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagagcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccaacaattaaatggatttctaggaaaagctaccttaagaaaggctggttaccatcggagtttacaaagtgctttcacgttcttacttgttgcattatacattcatgcatttctaggctagagaaccttctagatttgatgcttacaactattctgttgtgactatgagaacatttctgtctctagaagtcatctgtctgtattgatctttatgctatattactatctgtggttacggtggagacattgacattattactggagtcaagcccttataagtcaaaagcatctatgtgtcgtaaaacattcctcaaacattttttcatgcaaatacacacttctttccccaaacatcatgtagcacatcaatatgtagggagacattcttatgcatcatttggtttgttttataaccaattcattaaatgtaattcataaaatgtactatgaaaaaaattatacgctatgggatactggcaaaagtgcacatatttcataaccaaattagtagcaccagtcttaatttgatgtttttcaacttttattcattgagatgttttgaagcaattaggatatgtgtgtttactgtactttttgttttgatccgtttgtataaatgatagcaatatcttggacacatctgaaatacaaaatgtttttgtctaccaaagaaaaatgttgaaaaataagcaaatgtatacctagcaatcacttttactttttgtaattctgtctcttagaaaaatacataatctaatcaatttctttgttcatgcctatatactgtaaaatttaggtatactcaagactagtttaaagaatcaaagtcatttttttctctaataaactaccacaacctttcttttttaaaaaaaaaaa

SEQ ID NO: 75 - DNA, 3545 bp, Homo sapiens
  sequence of CXCL12b
gccgcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgtgagagggtcagacgcctgaggaacccttacagtaggagcccagctctgaaaccagtgttagggaagggcctgccacagcctcccctgccagggcagggccccaggcattgccaagggctttgttttgcacactttgccatattttcaccatttgattatgtagcaaaatacatgacatttatttttcatttagtttgattattcagtgtcactggcgacacgtagcagcttagactaaggccattattgtacttgccttattagagtgtctttccacggagccactcctctgactcagggctcctgggttttgtattctctgagctgtgcaggtggggagactgggctgagggagcctggccccatggtcagccctagggtggagagccaccaagagggacgcctgggggtgccaggaccagtcaacctgggcaaagcctagtgaaggcttctctctgtgggatgggatggtggagggccacatgggaggctcacccccttctccatccacatgggagccgggtctgcctcttctgggagggcagcagggctaccctgagctgaggcagcagtgtgaggccagggcagagtgagacccagccctcatcccgagcacctccacatcctccacgttctgctcatcattctctgtctcatccatcatcatgtgtgtccacgactgtctccatggccccgcaaaaggactctcaggaccaaagctttcatgtaaactgtgcaccaagcaggaaatgaaaatgtcttgtgttacctgaaaacactgtgcacatctgtgtcttgtttggaatattgtccattgtccaatcctatgtttttgttcaaagccagcgtcctcctctgtgaccaatgtcttgatgcatgcactgttccccctgtgcagccgctgagcgaggagatgctccttgggccctttgagtgcagtcctgatcagagccgtggtcctttggggtgaactaccttggttcccccactgatcacaaaaacatggtgggtccatgggcagagcccaagggaattcggtgtgcaccagggttgaccccagaggattgctgccccatcagtgctccctcacatgtcagtaccttcaaactagggccaagcccagcactgcttgaggaaaacaagcattcacaacttgtttttggtttttaaaacccagtccacaaaataaccaatcctggacatgaagattctttcccaattcacatctaacctcatcttcttcaccatttggcaatgccatcatctcctgccttcctcctgggccctctctgctctgcgtgtcacctgtgcttcgggcccttcccacaggacatttctctaagagaacaatgtgctatgtgaagagtaagtcaacctgcctgacatttggagtgttccccttccactgagggcagtcgatagagctgtattaagccacttaaaatgttcacttttgacaaaggcaagcacttgtgggtttttgttttgtttttcattcagtcttacgaatacttttgccctttgattaaagactccagttaaaaaaaattttaatgaagaaagtggaaaacaaggaagtcaaagcaaggaaactatgtaacatgtaggaagtaggaagtaaattatagtgatgtaatcttgaattgtaactgttcttgaatttaataatctgtagggtaattagtaacatgtgttaagtattttcataagtatttcaaattggagcttcatggcagaaggcaaacccatcaacaaaaattgtcccttaaacaaaaattaaaatcctcaatccagctatgttatattgaaaaaatagagcctgagggatctttactagttataaagatacagaactctttcaaaaccttttgaaattaacctctcactataccagtataattgagttttcagtggggcagtcattatccaggtaatccaagatattttaaaatctgtcacgtagaacttggatgtacctgcccccaatccatgaaccaagaccattgaattcttggttgaggaaacaaacatgaccctaaatcttgactacagtcaggaaaggaatcatttctatttctcctccatgggagaaaatagataagagtagaaactgcagggaaaattatttgcataacaattcctctactaacaatcagctccttcctggagactgcccagctaaagcaatatgcatttaaatacagtcttccatttgcaagggaaaagtctcttgtaatccgaatctctttttgctttcgaactgctagtcaagtgcgtccacgagctgtttactagggatccctcatctgtccctccgggacctggtgctgcctctacctgacactcccttgggctccctgtaacctcttcagaggccctcgctgccagctctgtatcaggacccagaggaaggggccagaggctcgttgactggctgtgtgttgggattgagtctgtgccacgtgtttgtgctgtggtgtgtccccctctgtccaggcactgagataccagcgaggaggctccagagggcactctgcttgttattagagattacctcctgagaaaaaaggttccgcttggagcagaggggctgaatagcagaaggttgcacctcccccaaccttagatgttctaagtctttccattggatctcattggacccttccatggtgtgatcgtctgactggtgttatcaccgtgggctccctgactgggagttgatcgcctttcccaggtgctacacccttttccagctggatgagaatttgagtgctctgatccctctacagagcttccctgactcattctgaaggagccccattcctgggaaatattccctagaaacttccaaatcccctaagcagaccactgataaaaccatgtagaaaatttgttattttgcaacctcgctggactctcagtctctgagcagtgaatgattcagtgttaaatgtgatgaatactgtattttgtattgtttcaattgcatctcccagataatgtgaaaatggtccaggagaaggccaattcctatacgcagcgtgctttaaaaaataaataagaaacaactctttgagaaacaacaatttctactttgaagtcataccaatgaaaaaatgtatatgcacttataattttcctaataaagttctgtactcaaatgtagccaccaa

SEQ ID NO: 76 - DNA, 1219 bp, Homo sapiens
  sequence of CXCL13
gagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccaggaaaaagaacttccccatacaaataagcatgagactatgtaaaaataaccttgcagaagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtggggggcggggccggggggactctggtatctaattctttaatgattcctataaatctaatgacattcaataaagttgagcaaacattttacttaaaaaaaaaaaaaaaaaa

SEQ ID NO: 77 - DNA, 1670 bp, Homo sapiens
  sequence of CXCR3-1
ccaaccacaagcaccaaagcagaggggcaggcagcacaccacccagcagccagagcaccagcccagccatggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaggccggaatccgggctcccctttcgcccacagtctgacttccccgcattccaggctcctccctccctctgccggctctggctctccccaatatcctcgctcccgggactcactggcagccccagcaccaccaggtctcccgggaagccaccctcccagctctgaggactgcaccattgctgctccttagctgccaagccccatcctgccgcccgaggtggctgcctggagccccactgcccttctcatttggaaactaaaacttcatcttccccaagtgcggggagtacaaggcatggcgtagagggtgctgccccatgaagccacagcccaggcctccagctcagcagtgactgtggccatggtccccaagacctctatatttgctcttttatttttatgtctaaaatcctgcttaaaacttttcaataaacaagatcgtcaggaccaaaaaaaaaaaaaaaaaaaaaaaaaaaaaaaaaaaaaaaaaaaaaaa

SEQ ID NO: 78 - DNA, 1914 bp, Homo sapiens
  sequence of CXCR3-2
ccaaccacaagcaccaaagcagaggggcaggcagcacaccacccagcagccagagcaccagcccagccatggtccttgaggggtccctgggccgatgggatcacgcagaagaatgcgagagaagcagcctttgagaagggaagtcactatcccagagcccaggctgagcggatggagttgaggaagtacggccctggaagactggcggggacagttataggaggagctgctcagagtaaatcacagactaaatcagactcaatcacaaaagagttcctgccaggcctttacacagccccttcctccccgttcccgccctcacaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaggccggaatccgggctcccctttcgcccacagtctgacttccccgcattccaggctcctccctccctctgccggctctggctctccccaatatcctcgctcccgggactcactggcagccccagcaccaccaggtctcccgggaagccaccctcccagctctgaggactgcaccattgctgctccttagctgccaagccccatcctgccgcccgaggtggctgcctggagccccactgcccttctcatttggaaactaaaacttcatcttccccaagtgcggggagtacaaggcatggcgtagagggtgctgccccatgaagccacagcccaggcctccagctcagcagtgactgtggccatggtccccaagacctctatatttgctcttttatttttatgtctaaaatcctgcttaaaacttttcaataaacaagatcgtcaggaccaaaaaaaaaaaaaaaaaaaaaaaaaaaaaaaaaaaaaaaaaaaaaaa

SEQ ID NO: 79 - DNA, 2919 bp, Homo sapiens
  sequence of CXCR5-1
aaaaaaaaaaagtgatgagttgtgaggcaggtcgcggccctactgcctcaggagacgatgcgcagctcatttgcttaaatttgcagctgacggctgccacctctctagaggcacctggcggggagcctctcaacataagacagtgaccagtctggtgactcacagccggcacagccatgaactacccgctaacgctggaaatggacctcgagaacctggaggacctgttctgggaactggacagattggacaactataacgacacctccctggtggaaaatcatctctgccctgccacagaggggcccctcatggcctccttcaaggccgtgttcgtgcccgtggcctacagcctcatcttcctcctgggcgtgatcggcaacgtcctggtgctggtgatcctggagcggcaccggcagacacgcagttccacggagaccttcctgttccacctggccgtggccgacctcctgctggtcttcatcttgccctttgccgtggccgagggctctgtgggctgggtcctggggaccttcctctgcaaaactgtgattgccctgcacaaagtcaacttctactgcagcagcctgctcctggcctgcatcgccgtggaccgctacctggccattgtccacgccgtccatgcctaccgccaccgccgcctcctctccatccacatcacctgtgggaccatctggctggtgggcttcctccttgccttgccagagattctcttcgccaaagtcagccaaggccatcacaacaactccctgccacgttgcaccttctcccaagagaaccaagcagaaacgcatgcctggttcacctcccgattcctctaccatgtggcgggattcctgctgcccatgctggtgatgggctggtgctacgtgggggtagtgcacaggttgcgccaggcccagcggcgccctcagcggcagaaggcagtcagggtggccatcctggtgacaagcatcttcttcctctgctggtcaccctaccacatcgtcatcttcctggacaccctggcgaggctgaaggccgtggacaatacctgcaagctgaatggctctctccccgtggccatcaccatgtgtgagttcctgggcctggcccactgctgcctcaaccccatgctctacactttcgccggcgtgaagttccgcagtgacctgtcgcggctcctgacgaagctgggctgtaccggccctgcctccctgtgccagctcttccctagctggcgcaggagcagtctctctgagtcagagaatgccacctctctcaccacgttctaggtcccagtgtccccttttattgctgcttttccttggggcaggcagtgatgctggatgctccttccaacaggagctgggatcctaagggctcaccgtggctaagagtgtcctaggagtatcctcatttggggtagctagaggaaccaacccccatttctagaacatccctgccagctcttctgccggccctggggctaggctggagcccagggagcggaaagcagctcaaaggcacagtgaaggctgtccttacccatctgcacccccctgggctgagagaacctcacgcacctcccatcctaatcatccaatgctcaagaaacaacttctacttctgcccttgccaacggagagcgcctgcccctcccagaacacactccatcagcttaggggctgctgacctccacagcttcccctctctcctcctgcccacctgtcaaacaaagccagaagctgagcaccaggggatgagtggaggttaaggctgaggaaaggccagctggcagcagagtgtggccttcggacaactcagtccctaaaaacacagacattctgccaggcccccaagcctgcagtcatcttgaccaagcaggaagctcagactggttgagttcaggtagctgcccctggctctgaccgaaacagcgctgggtccaccccatgtcaccggatcctgggtggtctgcaggcagggctgactctaggtgcccttggaggccagccagtgacctgaggaagcgtgaaggccgagaagcaagaaagaaacccgacagagggaagaaaagagctttcttcccgaaccccaaggagggagatggatcaatcaaacccggcggtcccctccgccaggcgagatggggtggggtggagaactcctagggtggctgggtccaggggatgggaggttgtgggcattgatggggaaggaggctggcttgtcccctcctcactcccttcccataagctatagacccgaggaaactcagagtcggaacggagaaaggtggactggaaggggcccgtgggagtcatctcaaccatcccctccgtggcatcaccttaggcagggaagtgtaagaaacacactgaggcagggaagtccccaggccccaggaagccgtgccctgcccccgtgaggatgtcactcagatggaaccgcaggaagctgctccgtgcttgtttgctcacctggggtgtgggaggcccgtccggcagttctgggtgctccctaccacctccccagcctttgatcaggtggggagtcagggacccctgcccttgtcccactcaagccaagcagccaagctccttgggaggccccactggggaaataacagctgtggctcacgtgagagtgtcttcacggcaggacaacgaggaagccctaagacgtcccttttttctctgagtatctcctcgcaagctgggtaatcgatgggggagtctgaagcagatgcaaagaggcaagaggctggattttgaattttctttttaataaaaaggcacctataaaacaggtcaatacagtacaggcagcacagagacccccggaacaagcctaaaaattgtttcaaaataaaaaccaagaagatgtcttcacatattgtaaaaaaaaaaaaaaaaa

SEQ ID NO: 80 - DNA, 2896 bp, Homo sapiens
  sequence of CXCR5-2
ccactctaaggaatgcggtccctttgacaggcgaaaaactgaagttggaaaagacaaagtgatttgttcaaaattgaaatttgaaacttgacatttggtcagtgggccctatgtaggaaaaaacctccaagagagctagggttcctctcagagaggaaagacaggtccttaggtcctcaccctcccgtctccttgcccttgcagttctgggaactggacagattggacaactataacgacacctccctggtggaaaatcatctctgccctgccacagaggggcccctcatggcctccttcaaggccgtgttcgtgcccgtggcctacagcctcatcttcctcctgggcgtgatcggcaacgtcctggtgctggtgatcctggagcggcaccggcagacacgcagttccacggagaccttcctgttccacctggccgtggccgacctcctgctggtcttcatcttgccctttgccgtggccgagggctctgtgggctgggtcctggggaccttcctctgcaaaactgtgattgccctgcacaaagtcaacttctactgcagcagcctgctcctggcctgcatcgccgtggaccgctacctggccattgtccacgccgtccatgcctaccgccaccgccgcctcctctccatccacatcacctgtgggaccatctggctggtgggcttcctccttgccttgccagagattctcttcgccaaagtcagccaaggccatcacaacaactccctgccacgttgcaccttctcccaagagaaccaagcagaaacgcatgcctggttcacctcccgattcctctaccatgtggcgggattcctgctgcccatgctggtgatgggctggtgctacgtgggggtagtgcacaggttgcgccaggcccagcggcgccctcagcggcagaaggcagtcagggtggccatcctggtgacaagcatcttcttcctctgctggtcaccctaccacatcgtcatcttcctggacaccctggcgaggctgaaggccgtggacaatacctgcaagctgaatggctctctccccgtggccatcaccatgtgtgagttcctgggcctggcccactgctgcctcaaccccatgctctacactttcgccggcgtgaagttccgcagtgacctgtcgcggctcctgacgaagctgggctgtaccggccctgcctccctgtgccagctcttccctagctggcgcaggagcagtctctctgagtcagagaatgccacctctctcaccacgttctaggtcccagtgtccccttttattgctgcttttccttggggcaggcagtgatgctggatgctccttccaacaggagctgggatcctaagggctcaccgtggctaagagtgtcctaggagtatcctcatttggggtagctagaggaaccaacccccatttctagaacatccctgccagctcttctgccggccctggggctaggctggagcccagggagcggaaagcagctcaaaggcacagtgaaggctgtccttacccatctgcacccccctgggctgagagaacctcacgcacctcccatcctaatcatccaatgctcaagaaacaacttctacttctgcccttgccaacggagagcgcctgcccctcccagaacacactccatcagcttaggggctgctgacctccacagcttcccctctctcctcctgcccacctgtcaaacaaagccagaagctgagcaccaggggatgagtggaggttaaggctgaggaaaggccagctggcagcagagtgtggccttcggacaactcagtccctaaaaacacagacattctgccaggcccccaagcctgcagtcatcttgaccaagcaggaagctcagactggttgagttcaggtagctgcccctggctctgaccgaaacagcgctgggtccaccccatgtcaccggatcctgggtggtctgcaggcagggctgactctaggtgcccttggaggccagccagtgacctgaggaagcgtgaaggccgagaagcaagaaagaaacccgacagagggaagaaaagagctttcttcccgaaccccaaggagggagatggatcaatcaaacccggcggtcccctccgccaggcgagatggggtggggtggagaactcctagggtggctgggtccaggggatgggaggttgtgggcattgatggggaaggaggctggcttgtcccctcctcactcccttcccataagctatagacccgaggaaactcagagtcggaacggagaaaggtggactggaaggggcccgtgggagtcatctcaaccatcccctccgtggcatcaccttaggcagggaagtgtaagaaacacactgaggcagggaagtccccaggccccaggaagccgtgccctgcccccgtgaggatgtcactcagatggaaccgcaggaagctgctccgtgcttgtttgctcacctggggtgtgggaggcccgtccggcagttctgggtgctccctaccacctccccagcctttgatcaggtggggagtcagggacccctgcccttgtcccactcaagccaagcagccaagctccttgggaggccccactggggaaataacagctgtggctcacgtgagagtgtcttcacggcaggacaacgaggaagccctaagacgtcccttttttctctgagtatctcctcgcaagctgggtaatcgatgggggagtctgaagcagatgcaaagaggcaagaggctggattttgaattttctttttaataaaaaggcacctataaaacaggtcaatacagtacaggcagcacagagacccccggaacaagcctaaaaattgtttcaaaataaaaaccaagaagatgtcttcacatattgtaaaaaaaaaaaaaaaaa

SEQ ID NO: 81 - DNA, 1119 bp, Homo sapiens
  sequence of CXCL1
cacagagcccgggccgcaggcacctcctcgccagctcttccgctcctctcacagccgccagacccgcctgctgagccccatggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgaccagaagggaggaggaagctcactggtggctgttcctgaaggaggccctgcccttataggaacagaagaggaaagagagacacagctgcagaggccacctggattgtgcctaatgtgtttgagcatcgcttaggagaagtcttctatttatttatttattcattagttttgaagattctatgttaatattttaggtgtaaaataattaagggtatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggcttaattattgatgttttcatagagaatataaaaataaagcacttatagaaaaaaaaaaaaaaaa

SEQ ID NO: 82 - DNA, 1234 bp, Homo sapiens
  sequence of CXCL2
gagctccgggaatttccctggcccgggactccgggctttccagccccaaccatgcataaaaggggttcgccgttctcggagagccacagagcccgggccacaggcagctccttgccagctctcctcctcgcacagccgctcgaaccgcctgctgagccccatggcccgcgccacgctctccgccgcccccagcaatccccggctcctgcgggtggcgctgctgctcctgctcctggtggccgccagccggcgcgcagcaggagcgcccctggccactgaactgcgctgccagtgcttgcagaccctgcagggaattcacctcaagaacatccaaagtgtgaaggtgaagtcccccggaccccactgcgcccaaaccgaagtcatagccacactcaagaatgggcagaaagcttgtctcaaccccgcatcgcccatggttaagaaaatcatcgaaaagatgctgaaaaatggcaaatccaactgaccagaaggaaggaggaagcttattggtggctgttcctgaaggaggccctgcccttacaggaacagaagaggaaagagagacacagctgcagaggccacctggattgcgcctaatgtgtttgagcatcacttaggagaagtcttctatttatttatttatttatttatttgtttgttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtgagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggacattttatgtctttcttgtaaggcatactgccttgtttaatgttaattatgcagtgtttccctctgtgttagagcagagaggtttcgatatttattgatgttttcacaaagaacaggaaaataaaatatttaaaaatataaaaaaaaaaaaaaaaaaaaaaaaaaaaaaaaa

SEQ ID NO: 83 - DNA, 1166 bp, Homo sapiens
  sequence of CXCL3
gctccgggaatttccctggcccggccgctccgggctttccagtctcaaccatgcataaaaagggttcgccgatcttggggagccacacagcccgggtcgcaggcacctccccgccagctctcccgcttctcgcacagcttcccgacgcgtctgctgagccccatggcccacgccacgctctccgccgcccccagcaatccccggctcctgcgggtggcgctgctgctcctgctcctggtggccgccagccggcgcgcagcaggagcgtccgtggtcactgaactgcgctgccagtgcttgcagacactgcagggaattcacctcaagaacatccaaagtgtgaatgtaaggtcccccggaccccactgcgcccaaaccgaagtcatagccacactcaagaatgggaagaaagcttgtctcaaccccgcatcccccatggttcagaaaatcatcgaaaagatactgaacaaggggagcaccaactgacaggagagaagtaagaagcttatcagcgtatcattgacacttcctgcagggtggtccctgcccttaccagagctgaaaatgaaaaagagaacagcagctttctagggacagctggaaaggacttaatgtgtttgactatttcttacgagggttctacttatttatgtatttatttttgaaagcttgtattttaatattttacatgctgttatttaaagatgtgagtgtgtttcatcaaacatagctcagtcctgattatttaattggaatatgatgggttttaaatgtgtcattaaactaatatttagtgggagaccataatgtgtcagccaccttgataaatgacagggtggggaactggagggtggggggattgaaatgcaagcaattagtggatcactgttagggtaagggaatgtatgtacacatctattttttatactttttttttaaaaaaagaatgtcagttgttatttattcaaattatctcacattatgtgttcaacatttttatgctgaagtttcccttagacattttatgtcttgcttgtagggcataatgccttgtttaatgtccattctgcagcgtttctctttcccttggaaaagagaatttatcattactgttacatttgtacaaatgacatgataataaaagttttatgaaaaaaaaaaaaaaa

SEQ ID NO: 84 - DNA, 476 bp, Homo sapiens
  sequence of CXCL4
ccatcgcactgagcactgagatcctgctggaagctctgccgcagcatgagctccgcagccgggttctgcgcctcacgccccgggctgctgttcctggggttgctgctcctgccacttgtggtcgccttcgccagcgctgaagctgaagaagatggggacctgcagtgcctgtgtgtgaagaccacctcccaggtccgtcccaggcacatcaccagcctggaggtgatcaaggccggaccccactgccccactgcccaactgatagccacgctgaagaatggaaggaaaatttgcttggacctgcaagccccgctgtacaagaaaataattaagaaacttttggagagttagctactagctgcctacgtgtgtgcatttgctatatagcatacttcttttttccagtttcaatctaactgtgaaagaacttctgatatttgtgttatccttatgattttaaataaacaaaataaatc

SEQ ID NO: 85 - DNA, 2475 bp, Homo sapiens
  sequence of CXCL5
gtgcagaaggcacgaggaagccacagtgctccggatcctccaatcttcgctcctccaatctccgctcctccacccagttcaggaacccgcgaccgctcgcagcgctctcttgaccactatgagcctcctgtccagccgcgcggcccgtgtccccggtccttcgagctccttgtgcgcgctgttggtgctgctgctgctgctgacgcagccagggcccatcgccagcgctggtcctgccgctgctgtgttgagagagctgcgttgcgtttgtttacagaccacgcaaggagttcatcccaaaatgatcagtaatctgcaagtgttcgccataggcccacagtgctccaaggtggaagtggtagcctccctgaagaacgggaaggaaatttgtcttgatccagaagccccttttctaaagaaagtcatccagaaaattttggacggtggaaacaaggaaaactgattaagagaaatgagcacgcatggaaaagtttcccagtcttcagcagagaagttttctggaggtctctgaacccagggaagacaagaaggaaagattttgttgttgtttgtttatttgtttttccagtagttagctttcttcctggattcctcactttgaagagtgtgaggaaaacctatgtttgccgcttaagctttcagctcagctaatgaagtgtttagcatagtacctctgctatttgctgttattttatctgctatgctattgaagttttggcaattgactatagtgtgagccaggaatcactggctgttaatctttcaaagtgtcttgaattgtaggtgactattatatttccaagaaatattccttaagatattaactgagaaggctgtggatttaatgtggaaatgatgtttcataagaattctgttgatggaaatacactgttatcttcacttttataagaaataggaaatattttaatgtttcttggggaatatgttagagaatttccttactcttgattgtgggatactatttaattatttcactttagaaagctgagtgtttcacaccttatctatgtagaatatatttccttattcagaatttctaaaagtttaagttctatgagggctaatatcttatcttcctataattttagacattctttatctttttagtatggcaaactgccatcatttacttttaaactttgattttatatgctatttattaagtattttattaggagtaccataattctggtagctaaatatatattttagatagatgaagaagctagaaaacaggcaaattcctgactgctagtttatatagaaatgtattcttttagtttttaaagtaaaggcaaacttaacaatgacttgtactctgaaagttttggaaacgtattcaaacaatttgaatataaatttatcatttagttataaaaatatatagcgacatcctcgaggccctagcatttctccttggataggggaccagagagagcttggaatgttaaaaacaaaacaaaacaaaaaaaaacaaggagaagttgtccaagggatgtcaattttttatccctctgtatgggttagattttccaaaatcataatttgaagaaggccagcatttatggtagaatatataattatatataaggtggccacgctggggcaagttccctccccactcacagctttggcccctttcacagagtagaacctgggttagaggattgcagaagacgagcggcagcggggagggcagggaagatgcctgtcgggtttttagcacagttcatttcactgggattttgaagcatttctgtctgaatgtaaagcctgttctagtcctggtgggacacactggggttgggggtgggggaagatgcggtaatgaaaccggttagtcagtgttgtcttaatatccttgataatgctgtaaagtttatttttacaaatatttctgtttaagctatttcacctttgtttggaaatccttcccttttaaagagaaaatgtgacacttgtgaaaaggcttgtaggaaagctcctccctttttttctttaaacctttaaatgacaaacctaggtaattaatggttgtgaatttctatttttgctttgtttttaatgaacatttgtctttcagaataggattctgtgataatatttaaatggcaaaaacaaaacataattttgtgcaattaacaaagctactgcaagaaaaataaaacatttcttggtaaaaacgtatgtatttatatattatatatttatatataatatatattatatatttagcattgctgagctttttagatgcctattgtgtatcttttaaaggttttgaccattttgttatgagtaattacatatatattacattcactatattaaaattgtacttttttactatgtgtctcattggttcatagtctttattttgtcctttgaataaacattaaaagatttctaaacttcaaaaaaaaaaaaaaaaaa

SEQ ID NO: 86 - DNA, 1677 bp, Homo sapiens
  sequence of CXCL6
accccttctttccacactgccccctgagttcagggaatttccccagcatcccaaagcttgagtttcctgccagtcgggagggatgaatgcagataaagggagtgcagaaggcacgaggaaaccaaagtgctctgtatcctccagtctccgcgcctccacccagctcaggaacccgcgaaccctctcttgaccactatgagcctcccgtccagccgcgcggcccgtgtcccgggtccttcgggctccttgtgcgcgctgctcgcgctgctgctcctgctgacgccgccggggcccctcgccagcgctggtcctgtctctgctgtgctgacagagctgcgttgcacttgtttacgcgttacgctgagagtaaaccccaaaacgattggtaaactgcaggtgttccccgcaggcccgcagtgctccaaggtggaagtggtagcctccctgaagaacgggaagcaagtttgtctggacccggaagccccttttctaaagaaagtcatccagaaaattttggacagtggaaacaagaaaaactgagtaacaaaaaagaccatgcatcataaaattgcccagtcttcagcggagcagttttctggagatccctggacccagtaagaataagaaggaagggttggtttttttccattttctacatggattccctactttgaagagtgtgggggaaagcctacgcttctccctgaagtttacagctcagctaatgaagtactaatatagtatttccactatttactgttattttacctgataagttattgaaccctttggcaattgaccatattgtgagcaaagaatcactggttattagtctttcaatgaatattgaattgaagataactattgtatttctatcatacattccttaaagtcttaccgaaaaggctgtggatttcgtatggaaataatgttttattagtgtgctgttgagggaggtatcctgttgttcttactcactcttctcataaaataggaaatattttagttctgtttcttggggaatatgttactctttaccctaggatgctatttaagttgtactgtattagaacactgggtgtgtcataccgttatctgtgcagaatatatttccttattcagaatttctaaaaatttaagttctgtaagggctaatatattctcttcctatggttttagacgtttgatgtcttcttagtatggcataatgtcatgatttactcattaaactttgattttgtatgctattttttcactataggatgactataattctggtcactaaatatacactttagatagatgaagaagcccaaaaacagataaattcctgattgctaatttacatagaaatgtattctcttggttttttaaataaaagcaaaattaacaatgatctgtgctctgaaagttttgaaaatatatttgaacaatttgaatataaattcatcatttagtcctcaaaatatatatagcattgctaagattttcagatatctattgtggatcttttaaaggttttgaccattttgttatgaggaattatacatgtatcacattcactatattaaaattgcacttttattttttcctgtgtgtcatgttggtttttggtacttgtattgtcatttggagaaacaataaaagatttctaaaccaaaaaaaaaaaaaaaaaaa

SEQ ID NO: 87 - DNA, 1307 bp, Homo sapiens
  sequence of CXCL7
acttatctgcagacttgtaggcagcaactcaccctcactcagaggtcttctggttctggaaacaactctagctcagccttctccaccatgagcctcagacttgataccaccccttcctgtaacagtgcgagaccacttcatgccttgcaggtgctgctgcttctgtcattgctgctgactgctctggcttcctccaccaaaggacaaactaagagaaacttggcgaaaggcaaagaggaaagtctagacagtgacttgtatgctgaactccgctgcatgtgtataaagacaacctctggaattcatcccaaaaacatccaaagtttggaagtgatcgggaaaggaacccattgcaaccaagtcgaagtgatagccacactgaaggatgggaggaaaatctgcctggacccagatgctcccagaatcaagaaaattgtacagaaaaaattggcaggtgatgaatctgctgattaatttgttctgtttctgccaaacttctttaactcccaggaagggtagaattttgaaaccttgattttctagagttctcatttattcaggatacctattcttactgtattaaaatttggatatgtgtttcattctgtctcaaaaatcacattttattctgagaaggttggttaaaagatggcagaaagaagatgaaaataaataagcctggtttcaaccctctaattcttgcctaaacattggactgtactttgcatttttttctttaaaaatttctattctaacacaacttggttgatttttcctggtctactttatggttattagacatactcatgggtattattagatttcataatggtcaatgataataggaattacatggagcccaacagagaatatttgctcaatacatttttgttaatatatttaggaacttaatggagtctctcagtgtcttagtcctaggatgtcttatttaaaatactccctgaaagtttattctgatgtttattttagccatcaaacactaaaataataaattggtgaatatgaatcttataaactgtggttagctggtttaaagtgaatatatttgccactagtagaacaaaaatagatgatgaaaatgaattaacatatctacatagttataattctatcattagaatgagccttataaataagtacaatataggacttcaaccttactagactcctaattctaaattctacttttttcatcaacagaactttcattcattttttaaaccctaaaacttatacccacactattcttacaaaaatattcacatgaaataaaaatttgctattga

SEQ ID NO: 88 - DNA, 1718 bp, Homo sapiens
  sequence of CXCL8
gagggtgcataagttctctagtagggtgatgatataaaaagccaccggagcactccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaaa

SEQ ID NO: 89 - DNA, 2344 bp, Homo sapiens
  sequence of CXCL16
ggtgcgtccgcgggtggctgccccgcaggtgcgcgcggccggggctggcggcgactctctccaccgggccgcccgggaggctcatgcagcgcggctgggtcccgcggcgcccggatcggggaagtgaaagtgcctcggaggaggagggccggtccggcagtgcagccgcctcacaggtcggcggacgggccaggcgggcggcctcctgaaccgaaccgaatcggctcctcgggccgtcgtcctcccgcccctcctcgcccgccgccggagttttctttcggtttcttccaagattcctggccttccctcgacggagccgggcccagtgcgggggcgcagggcgcgggagctccacctcctcggctttccctgcgtccagaggctggcatggcgcgggccgagtactgagcgcacggtcggggcacagcagggccggggggtgcagctggctcgcgcctcctctccggccgccgtctcctccggtccccggcgaaagccattgagacaccagctggacgtcacgcgccggagcatgtctgggagtcagagcgaggtggctccatccccgcagagtccgcggagccccgagatgggacgggacttgcggcccgggtcccgcgtgctcctgctcctgcttctgctcctgctggtgtacctgactcagccaggcaatggcaacgagggcagcgtcactggaagttgttattgtggtaaaagaatttcttccgactccccgccatcggttcagttcatgaatcgtctccggaaacacctgagagcttaccatcggtgtctatactacacgaggttccagctcctttcctggagcgtgtgtgggggcaacaaggacccatgggttcaggaattgatgagctgtcttgatctcaaagaatgtggacatgcttactcggggattgtggcccaccagaagcatttacttcctaccagccccccaatttctcaggcctcagagggggcatcttcagatatccacacccctgcccagatgctcctgtccaccttgcagtccactcagcgccccaccctcccagtaggatcactgtcctcggacaaagagctcactcgtcccaatgaaaccaccattcacactgcgggccacagtctggcagctgggcctgaggctggggagaaccagaagcagccggaaaaaaatgctggtcccacagccaggacatcagccacagtgccagtcctgtgcctcctggccatcatcttcatcctcaccgcagccctttcctatgtgctgtgcaagaggaggagggggcagtcaccgcagtcctctccagatctgccggttcattatatacctgtggcacctgactctaatacctgagccaagaatggaagcttgtgaggagacggactctatgttgcccaggctgttatggaactcctgagtcaagtgatcctcccaccttggcctctgaaggtgcgaggattataggcgtcacctaccacatccagcctacacgtatttgttaatatctaacataggactaaccagccactgccctctcttaggcccctcatttaaaaacggttatactataaaatctgcttttcacactgggtgataataacttggacaaattctatgtgtattttgttttgttttgctttgctttgttttgagacggagtctcgctctgtcatccaggctggagtgcagtggcatgatctcggctcactgcaacccccatctcccaggttcaagcgattctcctgcctcctcctgagtagctgggactacaggtgctcaccaccacacccggctaattttttgtatttttagtagagacggggtttcaccatgttgaccaggctggtctcgaactcctgacctggtgatctgcccacccaggcctcccaaagtgctgggattaaaggtgtgagccaccatgcctggccctatgtgtgttttttaactactaaaaattatttttgtaatgattgagtcttctttatggaaacaactggcctcagcccttgcgcccttactgtgattcctggcttcattttttgctgatggttccccctcgtcccaaatctctctcccagtacaccagttgttcctcccccacctcagccctctcctgcatcctcctgtacccgcaacgaaggcctgggctttcccaccctccctccttagcaggtgccgtgctgggacaccatacgggttggtttcacctcctcagtcccttgcctaccccagtgagagtctgatcttgtttttattgttattgcttttattattattgcttttattatcattaaaactctagttcttgttttgtctctccgaaaaaaaaaaaaaaaaaaaaaa

SEQ ID NO: 90 - DNA, 2502 bp, Homo sapiens
  sequence of CXCR1
tattcatcaagtgccctctagctgttaagtcactctgatctctgactgcagctcctactgttggacacacctggccggtgcttcagttagatcaaaccattgctgaaactgaagaggacatgtcaaatattacagatccacagatgtgggattttgatgatctaaatttcactggcatgccacctgcagatgaagattacagcccctgtatgctagaaactgagacactcaacaagtatgttgtgatcatcgcctatgccctagtgttcctgctgagcctgctgggaaactccctggtgatgctggtcatcttatacagcagggtcggccgctccgtcactgatgtctacctgctgaacctggccttggccgacctactctttgccctgaccttgcccatctgggccgcctccaaggtgaatggctggatttttggcacattcctgtgcaaggtggtctcactcctgaaggaagtcaacttctacagtggcatcctgctgttggcctgcatcagtgtggaccgttacctggccattgtccatgccacacgcacactgacccagaagcgtcacttggtcaagtttgtttgtcttggctgctggggactgtctatgaatctgtccctgcccttcttccttttccgccaggcttaccatccaaacaattccagtccagtttgctatgaggtcctgggaaatgacacagcaaaatggcggatggtgttgcggatcctgcctcacacctttggcttcatcgtgccgctgtttgtcatgctgttctgctatggattcaccctgcgtacactgtttaaggcccacatggggcagaagcaccgagccatgagggtcatctttgctgtcgtcctcatcttcctgctttgctggctgccctacaacctggtcctgctggcagacaccctcatgaggacccaggtgatccaggagagctgtgagcgccgcaacaacatcggccgggccctggatgccactgagattctgggatttctccatagctgcctcaaccccatcatctacgccttcatcggccaaaattttcgccatggattcctcaagatcctggctatgcatggcctggtcagcaaggagttcttggcacgtcatcgtgttacctcctacacttcttcgtctgtcaatgtctcttccaacctctgaaaaccatcgatgaaggaatatctcttctcagaaggaaagaataaccaacaccctgaggttgtgtgtggaaggtgatctggctctggacaggcactatctgggttttggggggacgctataggatgtggggaagttaggaactggtgtcttcaggggccacaccaaccttctgaggagctgttgaggtacctccaaggaccggcctttgcacctccatggaaacgaagcaccatcattcccgttgaacgtcacatctttaacccactaactggctaattagcatggccacatctgagccccgaatctgacattagatgagagaacagggctgaagctgtgtcctcatgagggctggatgctctcgttgaccctcacaggagcatctcctcaactctgagtgttaagcgttgagccaccaagctggtggctctgtgtgctctgatccgagctcaggggggtggttttcccatctcaggtgtgttgcagtgtctgctggagacattgaggcaggcactgccaaaacatcaacctgccagctggccttgtgaggagctggaaacacatgttccccttgggggtggtggatgaacaaagagaaagagggtttggaagccagatctatgccacaagaaccccctttacccccatgaccaacatcgcagacacatgtgctggccacctgctgagccccaagtggaacgagacaagcagcccttagcccttcccctctgcagcttccaggctggcgtgcagcatcagcatccctagaaagccatgtgcagccaccagtccattgggcaggcagatgttcctaataaagcttctgttccgtgcttgtccctgtggaagtatcttggttgtgacagagtcaagggtgtgtgcagcattgttggctgttcctgcagtagaatgggggcagcacctcctaagaaggcacctctctgggttgaagggcagtgttccctggggctttaactcctgctagaacagtctcttgaggcacagaaactcctgttcatgcccatacccctggccaaggaagatccctttgtccacaagtaaaaggaaatgctcctccagggagtctcagcttcaccctgaggtgagcatcatcttctgggttaggccttgcctaggcatagccctgcctcaagctatgtgagctcaccagtccctccccaaatgctttccatgagttgcagttttttcctagtctgttttccctccttggagacagggccctgtcggtttattcactgtatgtccttggtgcctggagcctactaaatgctcaataaataatgatcacaggaaaaaaaaaaaaaaaaaa

SEQ ID NO: 91 - DNA, 2880 bp, Homo sapiens
  sequence of CXCR2
aggttcaaaacattcagagacagaaggtggatagacaaatctccaccttcagactggtaggctcctccagaagccatcagacaggaagatgtgaaaatccccagcactcatcccagaatcactaagtggcacctgtcctgggccaaagtcccaggacagacctcattgttcctctgtgggaatacctccccaggagggcatcctggatttcccccttgcaacccaggtcagaagtttcatcgtcaaggttgtttcatcttttttttcctgtctaacagctctgactaccacccaaccttgaggcacagtgaagacatcggtggccactccaataacagcaggtcacagctgctcttctggaggtgtcctacaggtgaaaagcccagcgacccagtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagtagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacggggtgaaaccctgtctctactaaaaatacaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggtgtgaccactgcagaagacagtatggcagctttcctcaaaacttcagacatagaattaacacatgatcctgcaattccacttataggaattgacccacaagaaatgaaagcagggacttgaacccatatttgtacaccaatattcatagcagcttattcacaagacccaaaaggcagaagcaacccaaatgttcatcaatgaatgaatgaatggctaagcaaaatgtgatatgtacctaacgaagtatccttcagcctgaaagaggaatgaagtactcatacatgttacaacacggacgaaccttgaaaactttatgctaagtgaaataagccagacatcaacagataaatagtttatgattccacctacatgaggtactgagagtgaacaaatttacagagacagaaagcagaacagtgattaccagggactgaggggaggggagcatgggaagtgacggtttaatgggcacagggtttatgtttaggatgttgaaaaagttctgcagataaacagtagtgatagttgtaccgcaatgtgacttaatgccactaaattgacacttaaaaatggtttaaatggtcaattttgttatgtatattttatatcaatttaaaaaaaaacctgagccccaaaaggtattttaatcaccaaggctgattaaaccaaggctagaaccacctgcctatattttttgttaaatgatttcattcaatatcttttttttaataaaccatttttacttgggtgtttataaaaaaaaaaa

SEQ ID NO: 92 - DNA, 1912 bp, Homo sapiens
  sequence of CXCR4a
ttttttttcttccctctagtgggcggggcagaggagttagccaagatgtgactttgaaaccctcagcgtctcagtgcccttttgttctaaacaaagaattttgtaattggttctaccaaagaaggatataatgaagtcactatgggaaaagatggggaggagagttgtaggattctacattaattctcttgtgcccttagcccactacttcagaatttcctgaagaaagcaagcctgaattggttttttaaattgctttaaaaattttttttaactgggttaatgcttgctgaattggaagtgaatgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 93 - DNA, 1691 bp, Homo sapiens
  sequence of CXCR4b
aacttcagtttgttggctgcggcagcaggtagcaaagtgacgccgagggcctgagtgctccagtagccaccgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 94 - DNA, 1953 bp, Homo sapiens
  sequence of CXCR6
gcagaccttgcttcatgagcaagctcatctctggaacaaactggcaaagcatctctgctggtgttcatcagaacagacaccatggcagagcatgattaccatgaagactatgggttcagcagtttcaatgacagcagccaggaggagcatcaagacttcctgcagttcagcaaggtctttctgccctgcatgtacctggtggtgtttgtctgtggtctggtggggaactctctggtgctggtcatatccatcttctaccataagttgcagagcctgacggatgtgttcctggtgaacctacccctggctgacctggtgtttgtctgcactctgcccttctgggcctatgcaggcatccatgaatgggtgtttggccaggtcatgtgcaagagcctactgggcatctacactattaacttctacacgtccatgctcatcctcacctgcatcactgtggatcgtttcattgtagtggttaaggccaccaaggcctacaaccagcaagccaagaggatgacctggggcaaggtcaccagcttgctcatctgggtgatatccctgctggtttccttgccccaaattatctatggcaatgtctttaatctcgacaagctcatatgtggttaccatgacgaggcaatttccactgtggttcttgccacccagatgacactggggttcttcttgccactgctcaccatgattgtctgctattcagtcataatcaaaacactgcttcatgctggaggcttccagaagcacagatctctaaagatcatcttcctggtgatggctgtgttcctgctgacccagatgcccttcaacctcatgaagttcatccgcagcacacactgggaatactatgccatgaccagctttcactacaccatcatggtgacagaggccatcgcatacctgagggcctgccttaaccctgtgctctatgcctttgtcagcctgaagtttcgaaagaacttctggaaacttgtgaaggacattggttgcctcccttaccttggggtctcacatcaatggaaatcttctgaggacaattccaagactttttctgcctcccacaatgtggaggccaccagcatgttccagttataggccttgccagggtttcgagaagctgctctggaatttgcaagtcatggctgtgccctcttgatgtggtgaggcaggctttgtttatagcttgcgcattctcatggagaagttatcagacactctggctggtttggaatgcttcttctcaggcatgaacatgtactgttctcttcttgaacactcatgctgaaagcccaagtagggggtctaaaatttttaaggactttccttcctccatctccaagaatgctgaaaccaagggggatgacatgtgactcctatgatctcaggttctccttgattgggactggggctgaaggttgaagaggtgagcacggccaacaaagctgttgatggtaggtggcacactgggtgcccaagctcagaaggctcttctgactactgggcaaagagtgtagatcagagcagcagtgaaaacaagtgctggcaccaccaggcacctcacagaaatgagatcaggctctgcctcaccttggggcttgacttttgtataggtagatgttcagattgctttgattaatccagaataactagcaccagggactatgaatgggcaaaactgaattataagaggctgataattccagtggtccatggaatgcttgaaaaatgtgcaaaacagcgtttaagactgtaatgaatctaagcagcatttctgaagtggactctttggtggctttgcattttaaaaatgaaattttccaatgtctgccacacaaacgtatgtaaatgtatatacccacacacatacacacatatgtcatatattactagcatatgagtttcatagctaagaaataaaactgttaaagtctccaaact

SEQ ID NO: 95 - DNA, 542 bp, Homo sapiens
  sequence of CCL1
acagtggtgagctcttagcttcaccaggctcatcaaagctgctccaggaaggcccaagccagaccagaagacatgcagatcatcaccacagccctggtgtgcttgctgctagctgggatgtggccggaagatgtggacagcaagagcatgcaggtacccttctccagatgttgcttctcatttgcggagcaagagattcccctgagggcaatcctgtgttacagaaataccagctccatctgctccaatgagggcttaatattcaagctgaagagaggcaaagaggcctgcgccttggacacagttggatgggttcagaggcacagaaaaatgctgaggcactgcccgtcaaaaagaaaatgagcagatttctttccattgtgggctctggaaaccacatggcttcacctgtccccgaaactaccagccctacaccattccttctgccctgcttttgctaggtcacagaggatctgcttggtcttgataagctatgttgttgcactttaaacatttaaattatacaatcatcaacccccaac

SEQ ID NO: 96 - DNA, 760 bp, Homo sapiens
  sequence of CCL2
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaaaaaaaaaaaaa

SEQ ID NO: 97 - DNA, 813 bp, Homo sapiens
  sequence of CCL3
agctggtttcagacttcagaaggacacgggcagcagacagtggtcagtcctttcttggctctgctgacactcgagcccacattccgtcacctgctcagaatcatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcctgaggggtccagaagcttcgaggcccagcgacctcggtgggcccagtggggaggagcaggagcctgagccttgggaacatgcgtgtgacctccacagctacctcttctatggactggttgttgccaaacagccacactgtgggactcttcttaacttaaattttaatttatttatactatttagtttttgtaatttattttcgatttcacagtgtgtttgtgattgtttgctctgagagttcccctgtcccctcccccttccctcacaccgcgtctggtgacaaccgagtggctgtcatcagcctgtgtaggcagtcatggcaccaaagccaccagactgacaaatgtgtatcggatgcttttgttcagggctgtgatcggcctggggaaataataaagatgctcttttaaaaggtaaaaaaaaaaaaaaaaaaa

SEQ ID NO: 98 - DNA, 667 bp, Homo sapiens
  sequence of CCL4
agcacaggacacagctgggttctgaagcttctgagttctgcagcctcacctctgagaaaacctctttgccaccaataccatgaagctctgcgtgactgtcctgtctctcctcatgctagtagctgccttctgctctccagcgctctcagcaccaatgggctcagaccctcccaccgcctgctgcttttcttacactgcgaggaagcttcctcgcaactttgtggtagattactatgagaccagcagcctctgctcccagccagctgtggtattccaaaccaaaagaagcaagcaagtctgtgctgatcccagtgaatcctgggtccaggagtacgtgtatgacctggaactgaactgagctgctcagagacaggaagtcttcagggaaggtcacctgagcccggatgcttctccatgagacacatctcctccatactcaggactcctctccgcagttcctgtcccttctcttaatttaatcttttttatgtgccgtgttattgtattaggtgtcatttccattatttatattagtttagccaaaggataagtgtcccctatggggatggtccactgtcactgtttctctgctgttgcaaatacatggataacacatttgattctgtgtgttttcataataaaactttaaaataaaatgcagacagtt

SEQ ID NO: 99 - DNA, 1488 bp, Homo sapiens
  sequence of CCL4L1
cctcacctctgagaaaacctctttgccaccaataccatgaagctctgcgtgactgtcctgtctctcctcgtgctagtagctgccttctgctctctagcactctcagcaccaagtaagtctacttttgcagctgctatttcgagtcaaggtgtaggcagagtccttttttctagtcatggctggcaaacagtgggatctggggatgggacaaaaggcagctaggaagattgccatgtagtctgctgctaaatgtagagtctagtagatattcagtaacattcaagttcctattttcttaagaattagcaaccagcagaggaaaacgatgggctggaagtcagactgttgaattggctctgcctttaattatttgttcaagcaagcccctgtccctctctgtgccttggtttccccatctgtcatatgaagggagtgcgatgtgttctgagactgaatccagttccaatcttctagatttctttctcgttcttctctgaagatccactattcagaataagactcctgctcatgttaggtgggaatggatacaagggaccatatttggggttctggtagctccacagggatgctcaatgaagatgcaaaattagaagtcaaaataaacagctcccatgggcagtgttgatctcaccctggcctttcctttcagtgggctcagaccctcccaccgcctgctgcttttcttacaccgcgaggaagcttcctcgcaactttgtggtagattactatgagaccagcagcctctgctcccagccagctgtggtgtgagtatcaacccctggctgccctgggaggcaagggtgagggctggatttttaaagggggcctgttttggggagggggtgatgagcgctggggaggcagctctcagggctgaagccttccctgacagcagtgaggtcacaggtcatgaactcacttttcaagtgctgaaggcggctgagtggcagccgagacagaagggggttcctggggaggaagttattcagaggacagggaagcaggggaaggcagacaggtcccatgagatatggaccaattccttaaaccatgctagaaaaacatgtggaaaagtcactaccaggctggcagggaatggggcaatctattcatactgattgcaatgcccactggttcctaatctgggcaacccctggggcccacagctaaatccagtgagtggaagttacagggagtctgcttccagtgctgctcgaggaaggatcccatccaccagagctgccccacatggaccatggtcaggcagaggaagatgcctaccacaggcaagggataaagccagatgacctcaaaggtcccatgggattctaatctgtctgctccttgttctacagattccaaaccaaaagaggcaagcaagtctgcgctgaccccagtgagtcctgggtccaggagtacgtgtatgacctggaactgaactgagctgctcagagacaggaagtcttc

SEQ ID NO: 100 - DNA, 1237 bp, Homo sapiens
  sequence of CCL5
gctgcagaggattcctgcagaggatcaagacagcacgtggacctcgcacagcctctcccacaggtaccatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctaccacacagcagcagttacaaaaaccttccccaggctggacgtggtggctcacgcctgtaatcccagcactttgggaggccaaggtgggtggatcacttgaggtcaggagttcgagaccagcctggccaacatgatgaaaccccatctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaatacaaaaattagccgggcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgccagggctctgtgaccaggaaggaagtcagcatgcctctagaggcaaggaggggaggaacactgcactcttaagcttccgccgtctcaacccctcacaggagcttactggcaaacatgaaaaatcggcttaccattaaagttctcaatgcaaccataaaaaaaaaa

SEQ ID NO: 101 - DNA, 810 bp, Homo sapiens
  sequence of CCL7
agcagaggggctgagaccaaaccagaaacctccaattctcatgtggaagcccatgccctcaccctccaacatgaaagcctctgcagcacttctgtgtctgctgctcacagcagctgctttcagcccccaggggcttgctcagccagttgggattaatacttcaactacctgctgctacagatttatcaataagaaaatccctaagcagaggctggagagctacagaaggaccaccagtagccactgtccccgggaagctgtaatcttcaagaccaaactggacaaggagatctgtgctgaccccacacagaagtgggtccaggactttatgaagcacctggacaagaaaacccaaactccaaagctttgaacattcatgactgaactaaaaacaagccatgacttgagaaacaaataatttgtataccctgtcctttctcagagtggttctgagattattttaatctaattctaaggaatatgagctttatgtaataatgtgaatcatggtttttcttagtagattttaaaagttattaatattttaatttaatcttccatggattttggtgggttttgaacataaagccttggatgtatatgtcatctcagtgctgtaaaaactgtgggatgctcctcccttctctacctcatgggggtattgtataagtccttgcaagaatcagtgcaaagatttgctttaattgttaagatatgatgtccctatggaagcatattgttattatataattacatatttgcatatgtatgactcccaaattttcacataaaatagatttttgtaaaaaa

SEQ ID NO: 102 - DNA, 1351 bp, Homo sapiens
  sequence of CCL8
gtgatggagagcaccagcaaagccttagggcccatccctggcctcctgttacccacagaggggtaggcccttggctctcttccactatgacgtcagcttccattcttcctttcttatagacaattttccatttcaaggaaatcagagcccttaatagttcagtgaggtcactttgctgagcacaatcccatacccttcagcctctgctccacagagcctaagcaaaagatagaaactcacaacttccttgttttgttatctggaaattatcccaggatctggtgcttactcagcatattcaaggaaggtcttacttcattcttccttgattgtgaccatgcccaggctctctgctccctataaaaggcaggcagagccaccgaggagcagagaggttgagaacaacccagaaaccttcacctctcatgctgaagctcacacccttgccctccaagatgaaggtttctgcagcgcttctgtgcctgctgctcatggcagccactttcagccctcagggacttgctcagccagattcagtttccattccaatcacctgctgctttaacgtgatcaataggaaaattcctatccagaggctggagagctacacaagaatcaccaacatccaatgtcccaaggaagctgtgatcttcaagaccaaacggggcaaggaggtctgtgctgaccccaaggagagatgggtcagggattccatgaagcatctggaccaaatatttcaaaatctgaagccatgagccttcatacatggactgagagtcagagcttgaagaaaagcttatttattttccccaacctcccccaggtgcagtgtgacattattttattataacatccacaaagagattatttttaaataatttaaagcataatatttcttaaaaagtatttaattatatttaagttgttgatgttttaactctatctgtcatacatcctagtgaatgtaaaatgcaaaatcctggtgatgtgttttttgtttttgttttcctgtgagctcaactaagttcacggcaaaatgtcattgttctccctcctacctgtctgtagtgttgtggggtcctcccatggatcatcaaggtgaaacactttggtattctttggcaatcagtgctcctgtaagtcaaatgtgtgctttgtactgctgttgttgaaattgatgttactgtatataactatggaattttgaaaaaaaatttcaaaaagaaaaaaatatatataatttaaaactaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 103 - DNA, 925 bp, Homo sapiens
  sequence of CCL11
atgggcaaaggcttccctggaatctcccacactgtctgctccctataaaaggcaggcagatgggccagaggagcagagaggctgagaccaacccagaaaccaccacctctcacgccaaagctcacaccttcagcctccaacatgaaggtctccgcagcacttctgtggctgctgctcatagcagctgccttcagcccccaggggctcgctgggccagcttctgtcccaaccacctgctgctttaacctggccaataggaagataccccttcagcgactagagagctacaggagaatcaccagtggcaaatgtccccagaaagctgtgatcttcaagaccaaactggccaaggatatctgtgccgaccccaagaagaagtgggtgcaggattccatgaagtatctggaccaaaaatctccaactccaaagccataaataatcaccatttttgaaaccaaaccagagcctgagtgttgcctaatttgttttcccttcttacaatgcattctgaggtaacctcattatcagtccaaagggcatgggttttattatatatatatatttttttttttaaaaaaaaaacgtattgcatttaatttattgaggctttaaaacttatcctccatgaatatcagttatttttaaactgtaaagctttgtgcagattctttaccccctgggagccccaattcgatcccctgtcacgtgtgggcaatgttccccctctcctctcttcctccctggaatcttgtaaaggtcctggcaaagatgatcagtatgaaaatgtcattgttcttgtgaacccaaagtgtgactcattaaatggaagtaaatgttgttttaggaatacataaagtatgtgcatattttattatagtcactagttgtaatttttttgtgggaaatccacactgagctgaggggg

SEQ ID NO: 104 - DNA, 861 bp, Homo sapiens
  sequence of CCL13
aaaaggccggcggaacagccagaggagcagagaggcaaagaaacattgtgaaatctccaactcttaaccttcaacatgaaagtctctgcagtgcttctgtgcctgctgctcatgacagcagctttcaacccccagggacttgctcagccagatgcactcaacgtcccatctacttgctgcttcacatttagcagtaagaagatctccttgcagaggctgaagagctatgtgatcaccaccagcaggtgtccccagaaggctgtcatcttcagaaccaaactgggcaaggagatctgtgctgacccaaaggagaagtgggtccagaattatatgaaacacctgggccggaaagctcacaccctgaagacttgaactctgctacccctactgaaatcaagctggagtacgtgaaatgacttttccattctcctctggcctcctcttctatgctttggaatacttctaccataattttcaaataggatgcattcggttttgtgattcaaaatgtactatgtgttaagtaatattggctattatttgacttgttgctggtttggagtttatttgagtattgctgatcttttctaaagcaaggccttgagcaagtaggttgctgtctctaagcccccttcccttccactatgagctgctggcagtgggtttgtattcggttcccaggggttgagagcatgcctgtgggagtcatggacatgaagggatgctgcaatgtaggaaggagagctctttgtgaatgtgaggtgttgctaaatatgttattgtggaaagatgaatgcaatagtaggactgctgacattttgcagaaaatacattttatttaaaatctcctaaaaaaaaaaa

SEQ ID NO: 105 - DNA, 506 bp, Homo sapiens
  sequence of CCL14-1
tttctcaacaattcctcaccgcaggagcctctgaagctcccaccaggccagctctcctcccacaacagcttcccacagcatgaagatctccgtggctgccattcccttcttcctcctcatcaccatcgccctagggaccaagactgaatcctcctcacggggaccttaccacccctcagagtgctgcttcacctacactacctacaagatcccgcgtcagcggattatggattactatgagaccaacagccagtgctccaagcccggaattgtcttcatcaccaaaaggggccattccgtctgtaccaaccccagtgacaagtgggtccaggactatatcaaggacatgaaggagaactgagtgacccagaaggggtggcgaaggcacagctcagagacataaagagaagatgccaaggccccctcctccacccaccgctaactctcagccccagtcaccctcttggagcttccctgctttgaattaaagaccactcatgctcttc

SEQ ID NO: 106 - DNA, 579 bp, Homo sapiens
  sequence of CCL14-2
tttctcaacaattcctcaccgcaggagcctctgaagctcccaccaggccagctctcctcccacaacagcttcccacagcatgaagatctccgtggctgccattcccttcttcctcctcatcaccatcgccctagggaccaagactgaatcctcctcacaaactggggggaaaccgaaggttgttaaaatacagctaaagttggtggggggaccttaccacccctcagagtgctgcttcacctacactacctacaagatcccgcgtcagcggattatggattactatgagaccaacagccagtgctccaagcccggaattgtcttcatcaccaaaaggggccattccgtctgtaccaaccccagtgacaagtgggtccaggactatatcaaggacatgaaggagaactgagtgacccagaaggggtggcgaaggcacagctcagagacataaagagaagatgccaaggccccctcctccacccaccgctaactctcagccccagtcaccctcttggagcttccctgctttgaattaaagaccactcatgctcttcaaaaaaaaaaaaaaaaaaaaaaaaa

SEQ ID NO: 107 - DNA, 1080 bp, Homo sapiens
  sequence of CCL15
tctcggtctctcactctgccttatacccctcagttgaattatttcttctgaggaggcaagaactgaggctgctgcagactgatatggattcaccactgctaacacctcctggttggaactacaggaatagaactggaaagggaaaaaaggcagcattcaccacatcccaatcctgaatccaagagtctaagatagtcccccactcctatctcaggcttagaggattagattaatctcctggagggaagactcttccttgaaacatttttttttatctgcctgtagctattgggataattcgggaaatccacagggacagttcaagtcatctttgtcctctactttctgttgcactctcagccttgttctctttttagaaactgcatggtaactattatatagctaaagaagagcattctgacctctgccctctgccctgggacttcctggatcctcctcttcttataaatacaagggcagagctggtatcccggggagccaggaagcagtgagcccaggagtcctcggccagccctgcctgcccaccaggaggatgaaggtctccgtggctgccctctcctgcctcatgcttgttgctgtccttggatcccaggcccagttcataaatgatgcagagacagagttaatgatgtcaaagcttccactggaaaatccagtagttctgaacagctttcactttgctgctgactgctgcacctcctacatctcacaaagcatcccgtgttcactcatgaaaagttattttgaaacgagcagcgagtgctccaagccaggtgtcatattcctcaccaagaaggggcggcaagtctgtgccaaacccagtggtccgggagttcaggattgcatgaaaaagctgaagccctactcaatataataataaagagacaaaagaggccagccacccacctccaacacctcctgtgagtttcttggtctgaaatacttaaaaaatatatatattgttgtgtctggtaatgaaagtaatgcatctaataaagagtattcaattttttaactttgcttgagttttaagaggaaataaactaatataaaactgaa

SEQ ID NO: 108 - DNA, 1497 bp, Homo sapiens
  sequence of CCL16
cggaccaccagcaacagacaacatcttcattcggctctccctgaagctgtactgcctcgctgagaggatgaaggtctccgaggctgccctgtctctccttgtcctcatccttatcattacttcggcttctcgcagccagccaaaagttcctgagtgggtgaacaccccatccacctgctgcctgaagtattatgagaaagtgttgccaaggagactagtggtgggatacagaaaggccctcaactgtcacctgccagcaatcatcttcgtcaccaagaggaaccgagaagtctgcaccaaccccaatgacgactgggtccaagagtacatcaaggatcccaacctacctttgctgcctaccaggaacttgtccacggttaaaattattacagcaaagaatggtcaaccccagctcctcaactcccagtgatgaccaggctttagtggaagcccttgtttacagaagagaggggtaaacctatgaaaacaggggaagccttattaggctgaaactagccagtcacattgagagaagcagaacaatgatcaaaataaaggagaagtatttcgaatattttctcaatcttaggaggaaataccaaagttaagggacgtgggcagaggtacgctcttttatttttatatttatatttttatttttttgagatagggtcttactctgtcacccaggctggagtgcagtggtgtgatcttggctcacttgatcttggctcactgtaacctccacctcccaggctcaagtgatcctcccaccccagcctcctgagtagctgggactacaggcttgcgccaccacacctggctaatttttgtatttttggtagagacgggattctaccatgttgcccaggctggtctcaaactcgtgtgcccaagcaatccacctgcctcagccttccaaaagtgctgggattacaggcgtgagccaccacatccggccagtgcactcttaatacacagaaaaatatatttcacatccttctcctgctctctttcaattcctcacttcacaccagtacacaagccattctaaatacttagccagtttccagccttccagatgatctttgccctctgggtcttgacccattaagagccccatagaactcttgatttttcctgtccatctttatggatttttctggatctatattttcttcaattattctttcattttataatgcaactttttcataggaagtccggatgggaatattcacattaatcatttttgcagagactttgctagatcctctcatattttgtcttcctcagggtggcaggggtacagagagtgcctgattggaaaaaaaaaaaaaagagagagagagagaagaagaagaagaagagacacaaatctctacctcccatgttaagctttgcaggacagggaaagaaagggtatgagacacggctaggggtaaactcttagtccaaaacccaagcatgcaataaataaaactcccttatttgaca

SEQ ID NO: 109 - DNA, 615 bp, Homo sapiens
  sequence of CCL17
gctcagagagaagtgactttgagctcacagtgtcaccgcctgctgatgggagagctgaattcaaaaccagggtgtctccctgagcagagggacctgcacacagagactccctcctgggctcctggcaccatggccccactgaagatgctggccctggtcaccctcctcctgggggcttctctgcagcacatccacgcagctcgagggaccaatgtgggccgggagtgctgcctggagtacttcaagggagccattccccttagaaagctgaagacgtggtaccagacatctgaggactgctccagggatgccatcgtttttgtaactgtgcagggcagggccatctgttcggaccccaacaacaagagagtgaagaatgcagttaaatacctgcaaagccttgagaggtcttgaagcctcctcaccccagactcctgactgtctcccgggactacctgggacctccaccgttggtgttcaccgcccccaccctgagcgcctgggtccaggggaggccttccagggacgaagaagagccacagtgagggagatcccatccccttgtctgaactggagccatgggcacaaagggcccagattaaagtctttatcctc

SEQ ID NO: 110 - DNA, 793 bp, Homo sapiens
  sequence of CCL18
aggagttgtgagtttccaagccccagctcactctgaccacttctctgcctgcccagcatcatgaagggccttgcagctgccctccttgtcctcgtctgcaccatggccctctgctcctgtgcacaagttggtaccaacaaagagctctgctgcctcgtctatacctcctggcagattccacaaaagttcatagttgactattctgaaaccagcccccagtgccccaagccaggtgtcatcctcctaaccaagagaggccggcagatctgtgctgaccccaataagaagtgggtccagaaatacatcagcgacctgaagctgaatgcctgaggggcctggaagctgcgagggcccagtgaacttggtgggcccaggagggaacaggagcctgagccagggcaatggccctgccaccctggaggccacctcttctaagagtcccatctgctatgcccagccacattaactaactttaatcttagtttatgcatcatatttcattttgaaattgatttctattgttgagctgcattatgaaattagtattttctctgacatctcatgacattgtctttatcatcctttcccctttcccttcaactcttcgtacattcaatgcatggatcaatcagtgtgattagctttctcagcagacattgtgccatatgtatcaaatgacaaatctttattgaatggttttgctcagcaccaccttttaatatattggcagtacttattatataaaaggtaaaccagcattctcactgtgaaaaaaaaaaaaaaaaaaaaaaaa

SEQ ID NO: 111 - DNA, 684 bp, Homo sapiens
  sequence of CCL19
cattcccagcctcacatcactcacaccttgcatttcacccctgcatcccagtcgccctgcagcctcacacagatcctgcacacacccagacagctggcgctcacacattcaccgttggcctgcctctgttcaccctccatggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagttaacctatgaccgtgcagagggagcccggagtccgagtcaagcattgtgaattattacctaacctggggaaccgaggaccagaaggaaggaccaggcttccagctcctctgcaccagacctgaccagccaggacagggcctggggtgtgtgtgagtgtgagtgtgagcgagagggtgagtgtggtcagagtaaagctgctccacccccagattgcaatgctaccaataaagccgcctggtgtttacaactaa

SEQ ID NO: 112 - DNA, 851 bp, Homo sapiens
  sequence of CCL20-1
agaatataacagcactcccaaagaactgggtactcaacactgagcagatctgttctttgagctaaaaaccatgtgctgtaccaagagtttgctcctggctgctttgatgtcagtgctgctactccacctctgcggcgaatcagaagcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggcttttctggaatggaattggacatagcccaagaacagaaagaaccttgctggggttggaggtttcacttgcacatcatggagggtttagtgcttatctaatttgtgcctcactggacttgtccaattaatgaagttgattcatattgcatcatagtttgctttgtttaagcatcacattaaagttaaactgtattttatgttatttatagctgtaggttttctgtgtttagctatttaatactaattttccataagctattttggtttagtgcaaagtataaaattatatttgggggggaataagattatatggactttcttgcaagcaacaagctattttttaaaaaaaactatttaacattcttttgtttatattgttttgtctcctaaattgttgtaattgcattataaaataagaaaaatattaataagacaaatattgaaaataaagaaacaaaaagttcttctgttaaaaaaaaa

SEQ ID NO: 113 - DNA, 848 bp, Homo sapiens
  sequence of CCL20-2
agaatataacagcactcccaaagaactgggtactcaacactgagcagatctgttctttgagctaaaaaccatgtgctgtaccaagagtttgctcctggctgctttgatgtcagtgctgctactccacctctgcggcgaatcagaagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggcttttctggaatggaattggacatagcccaagaacagaaagaaccttgctggggttggaggtttcacttgcacatcatggagggtttagtgcttatctaatttgtgcctcactggacttgtccaattaatgaagttgattcatattgcatcatagtttgctttgtttaagcatcacattaaagttaaactgtattttatgttatttatagctgtaggttttctgtgtttagctatttaatactaattttccataagctattttggtttagtgcaaagtataaaattatatttgggggggaataagattatatggactttcttgcaagcaacaagctattttttaaaaaaaactatttaacattcttttgtttatattgttttgtctcctaaattgttgtaattgcattataaaataagaaaaatattaataagacaaatattgaaaataaagaaacaaaaagttcttctgttaaaaaaaaa

SEQ ID NO: 114 - DNA, 2933 bp, Homo sapiens
  sequence of CCL22
gcagacacctgggctgagacatacaggacagagcatggatcgcctacagactgcactcctggttgtcctcgtcctccttgctgtggcgcttcaagcaactgaggcaggcccctacggcgccaacatggaagacagcgtctgctgccgtgattacgtccgttaccgtctgcccctgcgcgtggtgaaacacttctactggacctcagactcctgcccgaggcctggcgtggtgttgctaaccttcagggataaggagatctgtgccgatcccagagtgccctgggtgaagatgattctcaataagctgagccaatgaagagcctactctgatgaccgtggccttggctcctccaggaaggctcaggagccctacctccctgccattatagctgctccccgccagaagcctgtgccaactctctgcattccctgatctccatccctgtggctgtcacccttggtcacctccgtgctgtcactgccatctcccccctgacccctctaacccatcctctgcctccctccctgcagtcagagggtcctgttcccatcagcgattcccctgcttaaacccttccatgactccccactgccctaagctgaggtcagtctcccaagcctggcatgtggccctctggatctgggttccatctctgtctccagcctgcccacttcccttcatgaatgttgggttctagctccctgttctccaaacccatactacacatcccacttctgggtctttgcctgggatgttgctgacacccagaaagtcccaccacctgcacatgtgtagccccaccagccctccaaggcattgctcgcccaagcagctggtaattccatttcatgtattagatgtcccctggccctctgtcccctcttaataaccctagtcacagtctccgcagattcttgggatttgggggttttctcccccacctctccactagttggaccaaggtttctagctaagttactctagtctccaagcctctagcatagagcactgcagacaggccctggctcagaatcagagcccagaaagtggctgcagacaaaatcaataaaactaatgtccctcccctctccctgccaaaaggcagttacatatcaatacagagactcaaggtcactagaaatgggccagctgggtcaatgtgaagccccaaatttgcccagattcacctttcttcccccactccctttttttttttttttttgagatggagtttcgctcttgtcacccacgctggagtgcaatggtgtggtcttggcttattgaagcctctgcctcctgggttcaagtgattctcttgcctcagcctcctgagtagctgggattacaggttcctgctaccacgcccagctaatttttgtatttttagtagagacgaggcttcaccatgttggccaggctggtctcgaactcctgtcctcaggtaatccgcccacctcagcctcccaaagtgctgggattacaggcgtgagccacagtgcctggcctcttccctctccccaccccccccccaacttttttttttttttatggcagggtctcactctgtcgcccaggctggagtgcagtggcgtgatctcggctcactacaacctcgacctcctgggttcaagcgattctcccaccccagcctcccaagtagctgggattacaggtgtgtgccactacggctggctaatttttgtatttttagtagagacaggtttcaccatattggccaggctggtcttgaactcctgacctcaagtgatccaccttccttgtgctcccaaagtgctgagattacaggcgtgagctatcacacccagcctccccctttttttcctaataggagactcctgtacctttcttcgttttacctatgtgtcgtgtctgcttacatttccttctcccctcaggctttttttgggtggtcctccaacctccaatacccaggcctggcctcttcagagtaccccccattccactttccctgcctccttccttaaatagctgacaatcaaattcatgctatggtgtgaaagactacctttgacttggtattataagctggagttatatatgtatttgaaaacagagtaaatacttaagaggccaaatagatgaatggaagaattttaggaactgtgagagggggacaaggtggagctttcctggccctgggaggaagctggctgtggtagcgtagcgctctctctctctgtctgtggcaggaggcaaagagtagggtgtaattgagtgaaggaatcctgggtagagaccattctcaggtggttgggccaggctaaagactgggatttgggtctatctatgcctttctggctgatttttgtagagacggggttttgccatgttacccaggctggtctcaaactcctgggctcaagcgatcctcctggctcagcctcccaaagtgctgggattacaggcgtgagtcactgcgcctggcttcctcttcctcttgagaaatattcttttcatacagcaagtatgggacagcagtgtcccaggtaaaggacataaatgttacaagtgtctggtcctttctgagggaggctggtgccgctctgcagggtatttgaacctgtggaattggaggaggccatttcactccctgaacccagcctgacaaatcacagtgagaatgttcaccttataggcttgctgtggggctcaggttgaaagtgtggggagtgacactgcctaggcatccagctcagtgtcatccagggcctgtgtccctcccgaacccagggtcaacctgcctaccacaggcactagaaggacgaatctgcctactgcccatgaacggggccctcaagcgtcctgggatctccttctccctcctgtcctgtccttgcccctcaggactgctggaaaataaatcctttaaaatagtaaaaaaaaaaaaaaa

SEQ ID NO: 115 - DNA, 603 bp, Homo sapiens
  sequence of CCL23-CKbeta8
ctggcatcccgagaagccaggaagcagtgagcccaggagtcctcggccagccctgcctgcccaccaggaggatgaaggtctccgtggctgccctctcctgcctcatgcttgttactgcccttggatcccaggcccgggtcacaaaagatgcagagacagagttcatgatgtcaaagcttccattggaaaatccagtacttctggacagattccatgctactagtgctgactgctgcatctcctacaccccacgaagcatcccgtgttcactcctggagagttactttgaaacgaacagcgagtgctccaagccgggtgtcatcttcctcaccaagaaggggcgacgtttctgtgccaaccccagtgataagcaagttcaggtttgcgtgagaatgctgaagctggacacacggatcaagaccaggaagaattgaacttgtcaaggtgaagggacacaagttgccagccaccaactttcttgcctcaactaccttcctgaattattttttaaagaagcatttattcttgtgttctggatttagagcaattcatctaataaacagtttctcactttaaaaaaaaaaaaaaaaaaaaaaaaaaaaa

SEQ ID NO: 116 - DNA, 641 bp, Homo sapiens
  sequence of CCL23-CKbeta8-1
ctggcatcccgagaagccaggaagcagtgagcccaggagtcctcggccagccctgcctgcccaccaggaggatgaaggtctccgtggctgccctctcctgcctcatgcttgttactgcccttggatcccaggcccgggtcacaaaagatgcagagacagagttcatgatgtcaaagcttccattggaaaatccagtacttctggacatgctctggaggagaaagattggtcctcagatgaccctttctcatgctgcaggattccatgctactagtgctgactgctgcatctcctacaccccacgaagcatcccgtgttcactcctggagagttactttgaaacgaacagcgagtgctccaagccgggtgtcatcttcctcaccaagaaggggcgacgtttctgtgccaaccccagtgataagcaagttcaggtttgcgtgagaatgctgaagctggacacacggatcaagaccaggaagaattgaacttgtcaaggtgaagggacacaagttgccagccaccaactttcttgcctcaactaccttcctgaattattttttaaagaagcatttattcttgtgttctggatttagagcaattcatctaataaacagtttctcactttaaaaaaaaaaaaaaaa

SEQ ID NO: 117 - DNA, 360 bp, Homo sapiens
  sequence of CCL24
atggcaggcctgatgaccatagtaaccagccttctgttccttggtgtctgtgcccaccacatcatccctacgggctctgtggtcatcccctctccctgctgcatgttctttgtttccaagagaattcctgagaaccgagtggtcagctaccagctgtccagcaggagcacatgcctcaaggcaggagtgatcttcaccaccaagaagggccagcagttctgtggcgaccccaagcaggagtgggtccagaggtacatgaagaacctggacgccaagcagaagaaggcttcccctagggccagggcagtggctgtcaagggccctgtccagagatatcctggcaaccaaaccacctgctaa

SEQ ID NO: 118 - DNA, 1002 bp, Homo sapiens
  sequence of CCL25-1
agatgggacagcttggcctacagcccggcgggcatcagctcccttgacccagtggatatcggtggccccgttattcgtccaggtgcccagggaggaggacccgcctgcagcatgaacctgtggctcctggcctgcctggtggccggcttcctgggagcctgggcccccgctgtccacacccaaggtgtctttgaggactgctgcctggcctaccactaccccattgggtgggctgtgctccggcgcgcctggacttaccggatccaggaggtgagcgggagctgcaatctgcctgctgcgatattctacctccccaagagacacaggaaggtgtgtgggaaccccaaaagcagggaggtgcagagagccatgaagctcctggatgctcgaaataaggtttttgcaaagctccaccacaacacgcagaccttccaagcaggccctcatgctgtaaagaagttgagttctggaaactccaagttatcatcgtccaagtttagcaatcccatcagcagcagtaagaggaatgtctccctcctgatatcagctaattcaggactgtgagccggctcatttctgggctccatcggcacaggaggggccggatctttctccgataaaaccgtcgccctacagacccagctgtccccacgcctctgtcttttgggtcaagtcttaatccctgcacctgagttggtcctccctctgcacccccaccacctcctgcccgtctggcaactggaaagagggagttggcctgattttaagccttttgccgctccggggaccagcagcaatcctgggcagccagtggctcttgtagagaagacttaggatacctctctcactttctgtttcttgccgtccaccccgggccatgccagtgtgtccctctgggtccctccaaaactctggtcagttcaaggatgcccctcccaggctatgcttttctataacttttaaataaaccttggggggtgatggagtcattcctgcctgtta

SEQ ID NO: 119 - DNA, 999 bp, Homo sapiens
  sequence of CCL25-2
agatgggacagcttggcctacagcccggcgggcatcagctcccttgacccagtggatatcggtggccccgttattcgtccaggtgcccagggaggaggacccgcctgcagcatgaacctgtggctcctggcctgcctggtggccggcttcctgggagcctgggcccccgctgtccacacccaaggtgtctttgaggactgctgcctggcctaccactaccccattgggtgggctgtgctccggcgcgcctggacttaccggatccaggaggtgagcgggagctgcaatctgcctgctgcgatattctacctccccaagagacacaggaaggtgtgtgggaaccccaaaagcagggaggtgcagagagccatgaagctcctggatgctcgaaataaggtttttgcaaagctccaccacaacacgcagaccttccaaggccctcatgctgtaaagaagttgagttctggaaactccaagttatcatcgtccaagtttagcaatcccatcagcagcagtaagaggaatgtctccctcctgatatcagctaattcaggactgtgagccggctcatttctgggctccatcggcacaggaggggccggatctttctccgataaaaccgtcgccctacagacccagctgtccccacgcctctgtcttttgggtcaagtcttaatccctgcacctgagttggtcctccctctgcacccccaccacctcctgcccgtctggcaactggaaagagggagttggcctgattttaagccttttgccgctccggggaccagcagcaatcctgggcagccagtggctcttgtagagaagacttaggatacctctctcactttctgtttcttgccgtccaccccgggccatgccagtgtgtccctctgggtccctccaaaactctggtcagttcaaggatgcccctcccaggctatgcttttctataacttttaaataaaccttggggggtgatggagtcattcctgcctgtta

SEQ ID NO: 120 - DNA, 562 bp, Homo sapiens
  sequence of CCL26
ctggaattgaggctgagccaaagaccccagggccgtctcagtctcataaaaggggatcaggcaggaggagtttgggagaaacctgagaagggcctgatttgcagcatcatgatgggcctctccttggcctctgctgtgctcctggcctccctcctgagtctccaccttggaactgccacacgtgggagtgacatatccaagacctgctgcttccaatacagccacaagccccttccctggacctgggtgcgaagctatgaattcaccagtaacagctgctcccagcgggctgtgatattcactaccaaaagaggcaagaaagtctgtacccatccaaggaaaaaatgggtgcaaaaatacatttctttactgaaaactccgaaacaattgtgactcagctgaattttcatccgaggacgcttggaccccgctcttggctctgcagccctctggggagcctgcggaatcttttctgaaggctacatggacccgctggggaggagagggtgtttcctcccagagttactttaataaaggttgttcatagagttgacttgttcat

SEQ ID NO: 121 - DNA, 469 bp, Homo sapiens
  sequence of CCL27
cccagataaaaggtaggggaggaggagagagagagaaggaagagtctaggctgagcaacatgaaggggcccccaaccttctgcagcctcctgctgctgtcattgctcctgagcccagaccctacagcagcattcctactgccacccagcactgcctgctgtactcagctctaccgaaagccactctcagacaagctactgaggaaggtcatccaggtggaactgcaggaggctgacggggactgtcacctccaggctttcgtgcttcacctggctcaacgcagcatctgcatccacccccagaaccccagcctgtcacagtggtttgagcaccaagagagaaagctccatgggactctgcccaagctgaattttgggatgctaaggaaaatgggctgaagcccccaatagccaaataataaagcagcattggataataatttctgaaaaaaaaaaaaaaaaaaaaaaaa

SEQ ID NO: 122 - DNA, 2689 bp, Homo sapiens
  sequence of CCR2-A
tttattctctggaacatgaaacattctgttgtgctcatatcatgcaaattatcactagtaggagagcagagagtggaaatgttccaggtataaagacccacaagataaagaagctcagagtcgttagaaacaggagcagatgtacagggtttgcctgactcacactcaaggttgcataagcaagatttcaaaattaatcctattctggagacctcaacccaatgtacaatgttcctgactggaaaagaagaactatatttttctgattttttttttcaaatctttaccattagttgccctgtatctccgccttcactttctgcaggaaactttatttcctacttctgcatgccaagtttctacctctagatctgtttggttcagttgctgagaagcctgacataccaggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctagagacagaaatgacagatctctgctttggaaatcacacgtctggcttcacagatgtgtgattcacagtgtgaatcttggtgtctacgttaccaggcaggaaggctgagaggagagagactccagctgggttggaaaacagtattttccaaactaccttccagttcctcatttttgaatacaggcatagagttcagactttttttaaatagtaaaaataaaattaaagctgaaaactgcaacttgtaaatgtggtaaagagttagtttgagttactatcatgtcaaacgtgaaaatgctgtattagtcacagagataattctagctttgagcttaagaattttgagcaggtggtatgtttgggagactgctgagtcaacccaatagttgttgattggcaggagttggaagtgtgtgatctgtgggcacattagcctatgtgcatgcagcatctaagtaatgatgtcgtttgaatcacagtatacgctccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctattttgccagtgggaactcctaaatcaagttggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcatatatttgtatgatcctaatgaatgcataaaatgttaagttgatggtgatgaaatgtaaatactgtttttaacaactatgatttggaaaataaatcaatgctataactatgttgaaaaaaaaaaaaaaaaaa

SEQ ID NO: 123 - DNA, 2335 bp, Homo sapiens
  sequence of CCR2-B
tttattctctggaacatgaaacattctgttgtgctcatatcatgcaaattatcactagtaggagagcagagagtggaaatgttccaggtataaagacccacaagataaagaagctcagagtcgttagaaacaggagcagatgtacagggtttgcctgactcacactcaaggttgcataagcaagatttcaaaattaatcctattctggagacctcaacccaatgtacaatgttcctgactggaaaagaagaactatatttttctgattttttttttcaaatctttaccattagttgccctgtatctccgccttcactttctgcaggaaactttatttcctacttctgcatgccaagtttctacctctagatctgtttggttcagttgctgagaagcctgacataccaggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaaggtatctctcggtgttcttccgaaagcacatcaccaagcgcttctgcaaacaatgtccagttttctacagggagacagtggatggagtgacttcaacaaacacgccttccactggggagcaggaagtctcggctggtttataaaacgaggagcagtttgattgttgtttataaagggagataacaatctgtatataacaacaaacttcaagggtttgttgaacaatagaaacctgtaaagcaggtgcccaggaacctcagggctgtgtgtactaatacagactatgtcacccaatgcatatccaacatgtgctcagggaataatccagaaaaactgtgggtagagactttgactctccagaaagctcatctcagctcctgaaaaatgcctcattaccttgtgctaatcctctttttctagtcttcataatttcttcactcaatctctgattctgtcaatgtcttgaaatcaagggccagctggaggtgaagaagagaatgtgacaggcacagatgaatgggagtgagggatagtggggtcagggctgagaggagaaggagggagacatgagcatggctgagcctggacaaagacaaaggtgagcaaagggctcacgcattcagccaggagatgatactggtccttagccccatctgccacgtgtatttaaccttgaagggttcaccaggtcagggagagtttgggaactgcaataacctgggagttttggtggagtccgatgattctcttttgcataagtgcatgacatatttttgctttattacagtttatctatggcacccatgcaccttacatttgaaatctatgaaatatcatgctccattgttcagatgcttcttaggccacatccccctgtctaaaaattcagaaaatttttgtttataaaaga

SEQ ID NO: 124 - DNA, 1796 bp, Homo sapiens
  sequence of CCR3-1
ctgatggtatctctgtttcaggagtggtgacgcctaagctatcactggacatatcaaggacttcactaaattagcaggtaccactggtcttcttgtgcttatccgggcaagaacttatcgaaatacaatagaagtttttacttagaagagattttcagcagatgagaagctggtaacagagaccaaaatagtttggagactaaagaatcattgcacatttcactgctgagttgtattggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaaca

SEQ ID NO: 125 - DNA, 1786 bp, Homo sapiens
  sequence of CCR3-2
ctgatggtatctctgtttcaggagtggtgacgcctaagctatcactggacatatcaaggacttcactaaattagcaggtaccactggtcttcttgtgcttatccgggcaagaacttatcgaaatacaatagaagtttttacttagaagagattttcagctgctgtggattggattatgccatttggaataagaatgctgttaagagcacacaagccaggttcctcaaggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaaca

SEQ ID NO: 126 - DNA, 1777 bp, Homo sapiens
  sequence of CCR3-3
ctgatggtatctctgtttcaggagtggtgacgcctaagctatcactggacatatcaaggacttcactaaattagcaggtaccactggtcttcttgtgcttatccgggcaagaacttatcgaaatacaatagaagtttttacttagaagagattttcagctgctgtggattggattatgccatttggaataagaatgctgttaagagcacacaagccagggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaaca

SEQ ID NO: 127 - DNA, 1015 bp, Homo sapiens
  sequence of CCR4
cctgaagggcctccaagggagacgcctagaagatggaggcccagattcttgagacgtttctctttctgatctagcaggagggacaaagagctcctccactccctcattccccaagaaggcccccagcctacccagtttccgtgaccattccgccctgggaaagcggcttcccagacctccttatctatttttcttgaatcatgagagatgaaattgcaacaacagttttctttgtcacaagattggtgaaaaaacatgataaactaagtaaacagcaaatagaagactttgcagaaaagctgatgacgatcttgtttgaaacatacagaagtcactggcactctgattgcccttctaaagggcaagccttcaggtgcatcaggataaacaacaatcagaataaagatcccattctagaaagggcatgtgtggaaagtaatgtagatttttctcacctgggacttccgaaggagatgaccatatgggtagatccctttgaagtatgctgtaggtatggtgagaaaaaccatccatttacagttgcttcttttaaaggcagatgggaggaatgggaactatatcaacaaatcagttatgccgttagtagagcctcatcagacgtttcctctggcacttcctgcgatgaagaaagttgtagcaaggaacctcgtgtcattcctaaagtcagcaatccgaagagtatttatcaggttgaaaacttgaaacagccctttcaatcttggttacaaatcccccgcaaaaagaatgtggtggacggccgtgttggcctcctgggaaacacttaccatggctcgcagaagcatcctaagtgttacaggcctgctatgcaccggctggacagaattttataacccacatctgggaatgaatttgcagcacctggtagaagaaggcaccttggaaggcactgccttgggcttccatggcaggaagatgagaagaaatcttcagggtgatttctggagcctgaaaagaataaaaaacaaaaccaaaa

SEQ ID NO: 128 - DNA, 3686 bp, Homo sapiens
  sequence of CCR5
cttcagatagattatatctggagtgaagaatcctgccacctatgtatctggcatagtattctgtgtagtgggatgagcagagaacaaaaacaaaataatccagtgagaaaagcccgtaaataaaccttcagaccagagatctattctctagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataaaaaaaaaaaaaaaaaaaaaaaaaaa

SEQ ID NO: 129 - DNA, 3693 bp, Homo sapiens
  sequence of CCR6
aactgtagtgcattttgccttctttccttcttagagtcacctctactttcctgctaccgctgcctgtgagctgaaggggctgaaccatacactcctttttctacaaccagcttgcattttttctgcccacaatgagcggggtaagatttttatttttggcaaggggtataatttgggttcactgtggctacttgaacactacactgcagctaactctatctttgtttcctttccaggaatcaatgaatttcagcgatgttttcgactccagtgaagattattttgtgtcagtcaatacttcatattactcagttgattctgagatgttactgtgctccttgcaggaggtcaggcagttctccaggctatttgtaccgattgcctactccttgatctgtgtctttggcctcctggggaatattctggtggtgatcacctttgctttttataagaaggccaggtctatgacagacgtctatctcttgaacatggccattgcagacatcctctttgttcttactctcccattctgggcagtgagtcatgccactggtgcgtgggttttcagcaatgccacgtgcaagttgctaaaaggcatctatgccatcaactttaactgcgggatgctgctcctgacttgcattagcatggaccggtacatcgccattgtacaggcgactaagtcattccggctccgatccagaacactaccgcgcagcaaaatcatctgccttgttgtgtgggggctgtcagtcatcatctccagctcaacttttgtcttcaaccaaaaatacaacacccaaggcagcgatgtctgtgaacccaagtaccagactgtctcggagcccatcaggtggaagctgctgatgttggggcttgagctactctttggtttctttatccctttgatgttcatgatattttgttacacgttcattgtcaaaaccttggtgcaagctcagaattctaaaaggcacaaagccatccgtgtaatcatagctgtggtgcttgtgtttctggcttgtcagattcctcataacatggtcctgcttgtgacggctgcaaatttgggtaaaatgaaccgatcctgccagagcgaaaagctaattggctatacgaaaactgtcacagaagtcctggctttcctgcactgctgcctgaaccctgtgctctacgcttttattgggcagaagttcagaaactactttctgaagatcttgaaggacctgtggtgtgtgagaaggaagtacaagtcctcaggcttctcctgtgccgggaggtactcagaaaacatttctcggcagaccagtgagaccgcagataacgacaatgcgtcgtccttcactatgtgatagaaagctgagtctccctaaggcatgtgtgaaacatactcatagatgttatgcaaaaaaaagtctatggccaggtatgcatggaaaatgtgggaattaagcaaaatcaagcaagcctctctcctgcgggacttaacgtgctcatgggctgtgtgatctcttcagggtggggtggtctctgataggtagcattttccagcactttgcaaggaatgttttgtagctctagggtatatatccgcctggcatttcacaaaacagcctttgggaaatgctgaattaaagtgaattgttgacaaatgtaaacattttcagaaatattcatgaagcggtcacagatcacagtgtcttttggttacagcacaaaatgatggcagtggtttgaaaaactaaaacagaaaaaaaaatggaagccaacacatcactcattttaggcaaatgtttaaacatttttatctatcagaatgtttattgttgctggttataagcagcaggattggccggctagtgtttcctctcatttccctttgatacagtcaacaagcctgaccctgtaaaatggaggtggaaagacaagctcaagtgttcacaacctggaagtgcttcggaaagaaggggacaatggcagaacaggtgttggtgacaattgtcaccaattggataaagcagctcaggttgtagtgggccattaggaaactgtcggtttgctttgatttccctgggagctgttctctgtcgtgagtgtctcttgtctaaacgtccattaagctgagagtgctatgaagacaggatctagaataatcttgctcacagctgtgctctgagtgcctagcggagttccagcaaacaaaatggactcaagagagatttgattaatgaatcgtaatgaagttggggtttattgtacagtttaaaatgttagatgtttttaattttttaaataaatggaatacttttttttttttttaaagaaagcaactttactgagacaatgtagaaagaagttttgttccgtttctttaatgtggttgaagagcaatgtgtggctgaagacttttgttatgaggagctgcagattagctaggggacagctggaattatgctggcttctgataattattttaaaggggtctgaaatttgtgatggaatcagattttaacagctctcttcaatgacatagaaagttcatggaactcatgtttttaaagggctatgtaaatatatgaacattagaaaaatagcaacttgtgttacaaaaatacaaacacatgttaggaaggtactgtcatgggctaggcatggtggctcacacctgtaatcccagcattttgggaagctaagatgggtggatcacttgaggtcaggagtttgagaccagcctggccaacatggcgaaacccctctctactaaaaatacaaaaatttgccaggcgtggtggcgggtgcctgtaatcccagctacttgggaggctgaggcaagagaatcgcttgaacccaggaggcagaggttgcagtgagccgagatcgtgccattgcactccagcctgggtgacaaagcgagactccatctcaaaaaaaaaaaaaaaaaaaaaaggaaagaactgtcatgtaaacataccgacatgtttaaacctgacaatggtgttatttgaaactttatattgttcttgtaagctttaactatatctctctttaaaatgcaaaataatgtcttaagattcaaagtctgtatttttaaagcatggctttggctttgcaaaataaaaaatgtgttttgtacatgaagtaggaatcgtatttcagcttcaaggttcagattgaggggcccactgtttggagaggatggtattcaggctttctcatgtccttcaaatctgttagcgtttgactctagaaatcaaagcaaaggagtggttacccagacacttcttttggtgtgatcaatgcgctgatgtgatctatgaagatgattcatgcttgaaaactagcacagaaacatcttgcttatttgccaaagctgggagatgagcttctctgcataatttaaatgttcagataaatgaagctgacttatttaagcaataaccttttaaacattttagctaagatgtataaaaatgtttccaaaatataccacatactttatttcttcttaaatgtagtacattaggttacatcatttttcttgctgtcttgggcatcaaaacaggtgccatggtaacctgacactctcaggagacattaagatagaaggggctgttcttcagtggttcccattgattctccccatatctttttgctctcaggctctggccgtctcttcctgagccttaactgtgt

SEQ ID NO: 130 - DNA, 1487 bp, Homo sapiens
  sequence of CCR8
tttgtagtgggaggatacctccagagaggctgctgctcattgagctgcactcacatgaggatacagactttgtgaagaaggaattggcaacactgaaacctccagaacaaaggctgtcactaaggtcccgctgccttgatggattatacacttgacctcagtgtgacaacagtgaccgactactactaccctgatatcttctcaagcccctgtgatgcggaacttattcagacaaatggcaagttgctccttgctgtcttttattgcctcctgtttgtattcagtcttctgggaaacagcctggtcatcctggtccttgtggtctgcaagaagctgaggagcatcacagatgtatacctcttgaacctggccctgtctgacctgctttttgtcttctccttcccctttcagacctactatctgctggaccagtgggtgtttgggactgtaatgtgcaaagtggtgtctggcttttattacattggcttctacagcagcatgtttttcatcaccctcatgagtgtggacaggtacctggctgttgtccatgccgtgtatgccctaaaggtgaggacgatcaggatgggcacaacgctgtgcctggcagtatggctaaccgccattatggctaccatcccattgctagtgttttaccaagtggcctctgaagatggtgttctacagtgttattcattttacaatcaacagactttgaagtggaagatcttcaccaacttcaaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagccaacagctgacttatgccacccatgtcacagaaatcatttcctttactcactgctgtgtgaaccctgttatctatgcttttgttggggagaagttcaagaaacacctctcagaaatatttcagaaaagttgcagccaaatcttcaactacctaggaagacaaatgcctagggagagctgtgaaaagtcatcatcctgccagcagcactcctcccgttcctccagcgtagactacattttgtgaggatcaatgaagactaaatataaaaaacattttcttgaatggcatgctagtagcagtgagcaaaggtgtgggtgtgaaaggtttccaaaaaaagttcagcatgaaggatgccatatatgttgttgccaacacttggaacacaatgactaaagacatagttgtgcatgcctggcacaacatcaagcctgtgattgtgtttattgatgatgttgaacaagtggtaactttaaaggattctgtatgccaagtgaaaaaaaaagatgtctgacctccttacatat

SEQ ID NO: 131 - DNA, 2544 bp, Homo sapiens
  sequence of CCR9A
gggtagctgcctgctcagaacccacaaagcctgcccctcatcccaggcagagagcaacccagctctttccccagacactgagagctggtggtgcctgctgtcccagggagagttgcatcgccctccacagagcaggcttgcatctgactgacccaccatgacacccacagacttcacaagccctattcctaacatggctgatgactatggctctgaatccacatcttccatggaagactacgttaacttcaacttcactgacttctactgtgagaaaaacaatgtcaggcagtttgcgagccatttcctcccacccttgtactggctcgtgttcatcgtgggtgccttgggcaacagtcttgttatccttgtctactggtactgcacaagagtgaagaccatgaccgacatgttccttttgaatttggcaattgctgacctcctctttcttgtcactcttcccttctgggccattgctgctgctgaccagtggaagttccagaccttcatgtgcaaggtggtcaacagcatgtacaagatgaacttctacagctgtgtgttgctgatcatgtgcatcagcgtggacaggtacattgccattgcccaggccatgagagcacatacttggagggagaaaaggcttttgtacagcaaaatggtttgctttaccatctgggtattggcagctgctctctgcatcccagaaatcttatacagccaaatcaaggaggaatccggcattgctatctgcaccatggtttaccctagcgatgagagcaccaaactgaagtcagctgtcttgaccctgaaggtcattctggggttcttccttcccttcgtggtcatggcttgctgctataccatcatcattcacaccctgatacaagccaagaagtcttccaagcacaaagccctaaaagtgaccatcactgtcctgaccgtctttgtcttgtctcagtttccctacaactgcattttgttggtgcagaccattgacgcctatgccatgttcatctccaactgtgccgtttccaccaacattgacatctgcttccaggtcacccagaccatcgccttcttccacagttgcctgaaccctgttctctatgtttttgtgggtgagagattccgccgggatctcgtgaaaaccctgaagaacttgggttgcatcagccaggcccagtgggtttcatttacaaggagagagggaagcttgaagctgtcgtctatgttgctggagacaacctcaggagcactctccctctgaggggtcttctctgaggtgcatggttcttttggaagaaatgagaaatacagaaacagtttccccactgatgggaccagagagagtgaaagagaaaagaaaactcagaaagggatgaatctgaactatatgattacttgtagtcagaatttgccaaagcaaatatttcaaaatcaactgactagtgcaggaggctgttgattggctcttgactgtgatgcccgcaattctcaaaggaggactaaggaccggcactgtggagcaccctggctttgccactcgccggagcatcaatgccgctgcctctggaggagcccttggattttctccatgcactgtgaacttctgtggcttcagttctcatgctgcctcttccaaaaggggacacagaagcactggctgctgctacagaccgcaaaagcagaaagtttcgtgaaaatgtccatctttgggaaattttctaccctgctcttgagcctgataacccatgccaggtcttatagattcctgatctagaacctttccaggcaatctcagacctaatttccttctgttctccttgttctgttctgggccagtgaaggtccttgttctgattttgaaacgatctgcaggtcttgccagtgaacccctggacaactgaccacacccacaaggcatccaaagtctgttggcttccaatccatttctgtgtcctgctggaggttttaacctagacaaggattccgcttattccttggtatggtgacagtgtctctccatggcctgagcagggagattataacagctgggttcgcaggagccagccttggccctgttgtaggcttgttctgttgagtggcacttgctttgggtccaccgtctgtctgctccctagaaaatgggctggttcttttggccctcttctttctgaggcccactttattctgaggaatacagtgagcagatatgggcagcagccaggtagggcaaaggggtgaagcgcaggccttgctggaaggctatttacttccatgcttctccttttcttactctatagtggcaacattttaaaagcttttaacttagagattaggctgaaaaaaataagtaatggaattcacctttgcatcttttgtgtctttcttatcatgatttggcaaaatgcatcacctttgaaaatatttcacatattggaaaagtgctttttaatgtgtatatgaagcattaattacttgtcactttctttaccctgtctcaatattttaagtgtgtgcaattaaagatcaaatagatacatt

SEQ ID NO: 132 - DNA, 2462 bp, Homo sapiens
  sequence of CCR9B
gcccctcatcccaggcagagagcaacccagctctttccccagacactgagagctggtggtgcctgctgtcccagggagagttgcatcgccctccacaagccctattcctaacatggctgatgactatggctctgaatccacatcttccatggaagactacgttaacttcaacttcactgacttctactgtgagaaaaacaatgtcaggcagtttgcgagccatttcctcccacccttgtactggctcgtgttcatcgtgggtgccttgggcaacagtcttgttatccttgtctactggtactgcacaagagtgaagaccatgaccgacatgttccttttgaatttggcaattgctgacctcctctttcttgtcactcttcccttctgggccattgctgctgctgaccagtggaagttccagaccttcatgtgcaaggtggtcaacagcatgtacaagatgaacttctacagctgtgtgttgctgatcatgtgcatcagcgtggacaggtacattgccattgcccaggccatgagagcacatacttggagggagaaaaggcttttgtacagcaaaatggtttgctttaccatctgggtattggcagctgctctctgcatcccagaaatcttatacagccaaatcaaggaggaatccggcattgctatctgcaccatggtttaccctagcgatgagagcaccaaactgaagtcagctgtcttgaccctgaaggtcattctggggttcttccttcccttcgtggtcatggcttgctgctataccatcatcattcacaccctgatacaagccaagaagtcttccaagcacaaagccctaaaagtgaccatcactgtcctgaccgtctttgtcttgtctcagtttccctacaactgcattttgttggtgcagaccattgacgcctatgccatgttcatctccaactgtgccgtttccaccaacattgacatctgcttccaggtcacccagaccatcgccttcttccacagttgcctgaaccctgttctctatgtttttgtgggtgagagattccgccgggatctcgtgaaaaccctgaagaacttgggttgcatcagccaggcccagtgggtttcatttacaaggagagagggaagcttgaagctgtcgtctatgttgctggagacaacctcaggagcactctccctctgaggggtcttctctgaggtgcatggttcttttggaagaaatgagaaatacagaaacagtttccccactgatgggaccagagagagtgaaagagaaaagaaaactcagaaagggatgaatctgaactatatgattacttgtagtcagaatttgccaaagcaaatatttcaaaatcaactgactagtgcaggaggctgttgattggctcttgactgtgatgcccgcaattctcaaaggaggactaaggaccggcactgtggagcaccctggctttgccactcgccggagcatcaatgccgctgcctctggaggagcccttggattttctccatgcactgtgaacttctgtggcttcagttctcatgctgcctcttccaaaaggggacacagaagcactggctgctgctacagaccgcaaaagcagaaagtttcgtgaaaatgtccatctttgggaaattttctaccctgctcttgagcctgataacccatgccaggtcttatagattcctgatctagaacctttccaggcaatctcagacctaatttccttctgttctccttgttctgttctgggccagtgaaggtccttgttctgattttgaaacgatctgcaggtcttgccagtgaacccctggacaactgaccacacccacaaggcatccaaagtctgttggcttccaatccatttctgtgtcctgctggaggttttaacctagacaaggattccgcttattccttggtatggtgacagtgtctctccatggcctgagcagggagattataacagctgggttcgcaggagccagccttggccctgttgtaggcttgttctgttgagtggcacttgctttgggtccaccgtctgtctgctccctagaaaatgggctggttcttttggccctcttctttctgaggcccactttattctgaggaatacagtgagcagatatgggcagcagccaggtagggcaaaggggtgaagcgcaggccttgctggaaggctatttacttccatgcttctccttttcttactctatagtggcaacattttaaaagcttttaacttagagattaggctgaaaaaaataagtaatggaattcacctttgcatcttttgtgtctttcttatcatgatttggcaaaatgcatcacctttgaaaatatttcacatattggaaaagtgctttttaatgtgtatatgaagcattaattacttgtcactttctttaccctgtctcaatattttaagtgtgtgcaattaaagatcaaatagatacat

SEQ ID NO: 133 - DNA, 1244 bp, Homo sapiens
  sequence of CCR10
agagatggggacggaggccacagagcaggtttcctggggccattactctggggatgaagaggacgcatactcggctgagccactgccggagctttgctacaaggccgatgtccaggccttcagccgggccttccaacccagtgtctccctgaccgtggctgcgctgggtctggccggcaatggcctggtcctggccacccacctggcagcccgacgcgcagcgcgctcgcccacctctgcccacctgctccagctggccctggccgacctcttgctggccctgactctgcccttcgcggcagcaggggctcttcagggctggagtctgggaagtgccacctgccgcaccatctctggcctctactcggcctccttccacgccggcttcctcttcctggcctgtatcagcgccgaccgctacgtggccatcgcgcgagcgctcccagccgggccgcggccctccactcccggccgcgcacacttggtctccgtcatcgtgtggctgctgtcactgctcctggcgctgcctgcgctgctcttcagccaggatgggcagcgggaaggccaacgacgctgtcgcctcatcttccccgagggcctcacgcagacggtgaagggggcgagcgccgtggcgcaggtggccctgggcttcgcgctgccgctgggcgtcatggtagcctgctacgcgcttctgggccgcacgctgctggccgccagggggcccgagcgccggcgtgcgctgcgcgtcgtggtggctctggtggcggccttcgtggtgctgcagctgccctacagcctcgccctgctgctggatactgccgatctactggctgcgcgcgagcggagctgccctgccagcaaacgcaaggatgtcgcactgctggtgaccagcggcttggccctcgcccgctgtggcctcaatcccgttctctacgccttcctgggcctgcgcttccgccaggacctgcggaggctgctacggggtgggagctcgccctcagggcctcaaccccgccgcggctgcccccgccggccccgcctttcttcctgctcagctcccacggagacccacagtctctcctgggacaactagggctgcgaatctagaggagggggcaggctgagggtcgtgggaaaggggagtaggtgggggaacactgagaaagaggcagggacctaaagggactacctctgtgccttgccacattaaattgataacatggaaatgaaaaaaaaaaaaaaaa

SEQ ID NO: 134 - DNA, 2224 bp, Homo sapiens
  sequence of CCRL1
aaaagtagctggagttaggtcatttgattttatactctgtactcaagactgctcctctctgccgactacaacagattggagccatggctttggaacagaaccagtcaacagattattattatgaggaaaatgaaatgaatggcacttatgactacagtcaatatgaactgatctgtatcaaagaagatgtcagagaatttgcaaaagttttcctccctgtattcctcacaatagttttcgtcattggacttgcaggcaattccatggtagtggcaatttatgcctattacaagaaacagagaaccaaaacagatgtgtacatcctgaatttggctgtagcagatttactccttctattcactctgcctttttgggctgttaatgcagttcatgggtgggttttagggaaaataatgtgcaaaataacttcagccttgtacacactaaactttgtctctggaatgcagtttctggcttgtatcagcatagacagatatgtggcagtaactaaagtccccagccaatcaggagtgggaaaaccatgctggatcatctgtttctgtgtctggatggctgccatcttgctgagcataccccagctggttttttatacagtaaatgacaatgctaggtgcattcccattttcccccgctacctaggaacatcaatgaaagcattgattcaaatgctagagatctgcattggatttgtagtaccctttcttattatgggggtgtgctactttatcacagcaaggacactcatgaagatgccaaacattaaaatatctcgacccctaaaagttctgctcacagtcgttatagttttcattgtcactcaactgccttataacattgtcaagttctgccgagccatagacatcatctactccctgatcaccagctgcaacatgagcaaacgcatggacatcgccatccaagtcacagaaagcatcgcactctttcacagctgcctcaacccaatcctttatgtttttatgggagcatctttcaaaaactacgttatgaaagtggccaagaaatatgggtcctggagaagacagagacaaagtgtggaggagtttccttttgattctgagggtcctacagagccaaccagtacttttagcatttaaaggtaaaactgctctgccttttgcttggatacatatgaatgatgctttcccctcaaataaaacatctgcattattctgaaactcaaatctcagacgccgtggttgcaacttataataaagaatgggttgggggaagggggagaaataaaagccaagaagaggaaacaagataataaatgtacaaaacatgaaaattaaaatgaacaatataggaaaataattgtaacaggcataagtgaataacactctgctgtaacgaagaagagctttgtggtgataattttgtatcttggttgcagtggtgcttatacaaatctacacaagtgataaaatgacacagaactatatacacacattgtaccaatttcaatttcctggttttgacattatagtataattatgtaagatggaaccattggggaaaactgggtgaagggtacccaggaccactctgtaccatctttgtaacttcctgtgaatttataataatttcaaaataaaacaagttaaaaaaaaacccactatgctataagttaggccatctaaaacagattattaaagaggttcatgttaaaaggcatttataattatttttaattatctaagttttaatacaagaacgatttccctgcataattttagtacttgaataagtatgcagcagaactccaactatcttttttcctgttttttttaaatttgtaagtaattttataaaatccacctcctccaaaaaagcaataaaaaaaaaacaaactataataagcttttctgattcttttcaaaacattcctggtaagttcctaaagacataatttgcttctatgatgtcaactttcttactaataactggttatcatgacaaatgttaggtttatcatatatagtctaggtgtaatcctcagactatcattttcatctgggttccaatttcttaacttcctaaagaattcatctgtttatacaagtctaccactgccgattgactaaaaaatacattatcccatgcataaaatgtcctattttcatttaaacactttatttttgagtaataaaaatatgtaccacaataaattattgttaattaa

SEQ ID NO: 135 - DNA, 1745 bp, Homo sapiens
  sequence of CCRL2-1
gaggaggaaacaacttcccggttgctttcagacgcttcagagatcctctggaggcctgggggagcttttgagtactttatttcagttggtccctgagctcggtgagtggggcgggtagagccaccaggggaatcaacagtggtttctcgtgcccctcagggtcaggagcagtctgatcaaaaggagggcatccactgtccggggccattcccacagctcccggatgctgggtctggaggctgcgcccttcccctgcaggagctcagcccagtgggcagtctgaagatggccaattacacgctggcaccagaggatgaatatgatgtcctcatagaaggtgaactggagagcgatgaggcagagcaatgtgacaagtatgacgcccaggcactctcagcccagctggtgccatcactctgctctgctgtgtttgtgatcggtgtcctggacaatctcctggttgtgcttatcctggtaaaatataaaggactcaaacgcgtggaaaatatctatcttctaaacttggcagtttctaacttgtgtttcttgcttaccctgcccttctgggctcatgctgggggcgatcccatgtgtaaaattctcattggactgtacttcgtgggcctgtacagtgagacatttttcaattgccttctgactgtgcaaaggtacctagtgtttttgcacaagggaaactttttctcagccaggaggagggtgccctgtggcatcattacaagtgtcctggcatgggtaacagccattctggccactttgcctgaattcgtggtttataaacctcagatggaagaccagaaatacaagtgtgcatttagcagaactcccttcctgccagctgatgagacattctggaagcattttctgactttaaaaatgaacatttcggttcttgtcctccccctatttatttttacatttctctatgtgcaaatgagaaaaacactaaggttcagggagcagaggtatagccttttcaagcttgtttttgccataatggtagtcttccttctgatgtgggcgccctacaatattgcatttttcctgtccactttcaaagaacacttctccctgagtgactgcaagagcagctacaatctggacaaaagtgttcacatcactaaactcatcgccaccacccactgctgcatcaaccctctcctgtatgcgtttcttgatgggacatttagcaaatacctctgccgctgtttccatctgcgtagtaacaccccacttcaacccagggggcagtctgcacaaggcacatcgagggaagaacctgaccattccaccgaagtgtaaactagcatccaccaaatgcaagaagaataaacatggattttcatctttctgcattatttcatgtaaattttctacacatttgtatacaaaatcggatacaggaagaaaagggagaggtgagctaacatttgctaagcactgaatttgtctcaggcaccgtgcaaggctctttacaaacgtgagctccttcgcctcctaccacttgtccatagtgtggataggactagtctcatttctctgagaagaaaactaaggcgcggaaatttgtctaagatcacataactaggaagtggcagaactgattctccagccctggtagcatttgctcagagcctacgcttggtccagaacatcaaactccaaaccctggggacaaacgacatgaaataaatgtattttaaaacatctaaaaaaaaaaaaaaaaa

SEQ ID NO: 136 - DNA, 1612 bp, Homo sapiens
  sequence of CCRL2-2
gagcatggtttcacttttgcaaacatttatttatacccttcgagagaaaaacgtctcagctgtcacaggaagctgcttcggggggtgagcaaactttttaaaatgcagaaattatgatctacacccgtttcttaaaaggcagtctgaagatggccaattacacgctggcaccagaggatgaatatgatgtcctcatagaaggtgaactggagagcgatgaggcagagcaatgtgacaagtatgacgcccaggcactctcagcccagctggtgccatcactctgctctgctgtgtttgtgatcggtgtcctggacaatctcctggttgtgcttatcctggtaaaatataaaggactcaaacgcgtggaaaatatctatcttctaaacttggcagtttctaacttgtgtttcttgcttaccctgcccttctgggctcatgctgggggcgatcccatgtgtaaaattctcattggactgtacttcgtgggcctgtacagtgagacatttttcaattgccttctgactgtgcaaaggtacctagtgtttttgcacaagggaaactttttctcagccaggaggagggtgccctgtggcatcattacaagtgtcctggcatgggtaacagccattctggccactttgcctgaattcgtggtttataaacctcagatggaagaccagaaatacaagtgtgcatttagcagaactcccttcctgccagctgatgagacattctggaagcattttctgactttaaaaatgaacatttcggttcttgtcctccccctatttatttttacatttctctatgtgcaaatgagaaaaacactaaggttcagggagcagaggtatagccttttcaagcttgtttttgccataatggtagtcttccttctgatgtgggcgccctacaatattgcatttttcctgtccactttcaaagaacacttctccctgagtgactgcaagagcagctacaatctggacaaaagtgttcacatcactaaactcatcgccaccacccactgctgcatcaaccctctcctgtatgcgtttcttgatgggacatttagcaaatacctctgccgctgtttccatctgcgtagtaacaccccacttcaacccagggggcagtctgcacaaggcacatcgagggaagaacctgaccattccaccgaagtgtaaactagcatccaccaaatgcaagaagaataaacatggattttcatctttctgcattatttcatgtaaattttctacacatttgtatacaaaatcggatacaggaagaaaagggagaggtgagctaacatttgctaagcactgaatttgtctcaggcaccgtgcaaggctctttacaaacgtgagctccttcgcctcctaccacttgtccatagtgtggataggactagtctcatttctctgagaagaaaactaaggcgcggaaatttgtctaagatcacataactaggaagtggcagaactgattctccagccctggtagcatttgctcagagcctacgcttggtccagaacatcaaactccaaaccctggggacaaacgacatgaaataaatgtattttaaaacatctaaaaaaaaaaaaaaaaaaaa

SEQ ID NO: 137 - DNA, 1367 bp, Homo sapiens
  sequence of XCL1
taagaaaaataaaagcatcagtattgcaaagactttccatgatcctacacccacctcgaaagccccctctcaccacaggaagtgcactgaccactggaggcataaaagaggtcctcaaagagcccgatcctcactctccttgcacagctcagcaggacctcagccatgagacttctcatcctggccctccttggcatctgctctctcactgcatacattgtggaaggtgtagggagtgaagtctcagataagaggacctgtgtgagcctcactacccagcgactgccggttagcagaatcaagacctacaccatcacggaaggctccttgagagcagtaatttttattaccaaacgtggcctaaaagtctgtgctgatccacaagccacatgggtgagagacgtggtcaggagcatggacaggaaatccaacaccagaaataacatgatccagaccaagccaacaggaacccagcaatcgaccaatacagctgtgactctgactggctagtagtctctggcaccctgtccgtctccagccagccagctcatttcactttacacgctcatggactgagtttatactcaccttttatgaaagcactgcatgaataaaattattcctttgtatttttacttttaaatgtcttctgtattcacttatatgttctaattaataaattatttattattaagaatagttccctagtctattcattatatttagggaaaggtagtgtatcattgttgtttgatttctgaccttgtacctctctttgatggtaaccataatggaagagattctggctagtgtctatcagaggtgaaagctatatcaatctctcttagagtccagcttgtaatggttctttacacatcagtcacaagttacagctgtgacaatggcaacaatttgagatgtatttcaacttgtctctataatagaattctgtttatagaataagggagaaaataatccagtcttcactgggttcccattctgagggtccactactcaaaaatttgcttcactcaatttttttcacctctttgtgttttattttggtgtcctattaaaggaataaaatgacacaacttgtcccttttttgtcccattagcaaaaattagaattttggtataaagaaactttattcaagtaaaaatcaataccctttgaattggacaataatctcactaccttattaggatttctgtatttgccattacgctagttatcatgcatgttatgctttactgcgaataagcttttaatgctccaaatgctgacccatgcaatatttcctcatgtgatcacaatttgcagtaaacttttaattaaatgctcatctggtaactcaacaccccag

SEQ ID NO: 138 - DNA, 1373 bp, Homo sapiens
  sequence of XCR1
ctttaagtgaaatatgaaacagagaagcaccatttctgcctcaagacgcatgtaaagaggtgtagattcaggttggagtgcagtggcatgatcacagctctctgcagtctcgccctcctgggctcaagcaatctttccccaccccccggtttcctgagtagctgggactataggcatgcgccaccacacccggatgctctaaacgtccctgccatctggtccagatggagtcctcaggcaacccagagagcaccacctttttttactatgaccttcagagccagccgtgtgagaaccaggcctgggtctttgctaccctcgccaccactgtcctatactgcctggtgtttctcctcagcctagtgggcaacagcctggtcctgtgggtcctggtgaagtatgagagcctggagtccctcaccaacatcttcatcctcaacctgtgcctctcagacctggtgttcgcctgcttgttgcctgtgtggatctccccataccactggggctgggtgctgggagacttcctctgcaaactcctcaatatgatcttctccatcagcctctacagcagcatcttcttcctgaccatcatgaccatccaccgctacctgtcggtagtgagccccctctccaccctgcgcgtccccaccctccgctgccgggtgctggtgaccatggctgtgtgggtagccagcatcctgtcctccatcctcgacaccatcttccacaaggtgctttcttcgggctgtgattattccgaactcacgtggtacctcacctccgtctaccagcacaacctcttcttcctgctgtccctggggattatcctgttctgctacgtggagatcctcaggaccctgttccgctcacgctccaagcggcgccaccgcacggtcaagctcatcttcgccatcgtggtggcctacttcctcagctggggtccctacaacttcaccctgtttctgcagacgctgtttcggacccagatcatccggagctgcgaggccaaacagcagctagaatacgccctgctcatctgccgcaacctcgccttctcccactgctgctttaacccggtgctctatgtcttcgtgggggtcaagttccgcacacacctgaaacatgttctccggcagttctggttctgccggctgcaggcacccagcccagcctcgatcccccactcccctggtgccttcgcctatgagggcgcctccttctactgaggggcctgtggcggtgcaggcgcaggtgcaggtggacagggactggaatgggggtcatggagaagcgggcctggaaggagcattgcagaacacagcagggtggagacgtctcctccgctgcaggcgtgcagtgaaggtcattcatta

SEQ ID NO: 139 - DNA, 3160 bp, Homo sapiens
  sequence of CX3CR1a
ggggcgggccgtgcttaccaggccgtggacttaaaccaggatgagagaacccctggaggcgtttaagttggcagacttggatttcaggaagagctctctggcttctgggtggagaatggccagtggggccttcaccatggatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtcctccaggaaatctggcccgtgctccgcaatgtggaaacaaattttcttggcttcctactccccctgctcattatgagttattgctacttcagaatcatccagacgctgttttcctgcaagaaccacaagaaagccaaagccattaaactgatccttctggtggtcatcgtgtttttcctcttctggacaccctacaacgttatgattttcctggagacgcttaagctctatgacttctttcccagttgtgacatgaggaaggatctgaggctggccctcagtgtgactgagacggttgcatttagccattgttgcctgaatcctctcatctatgcatttgctggggagaagttcagaagatacctttaccacctgtatgggaaatgcctggctgtcctgtgtgggcgctcagtccacgttgatttctcctcatctgaatcacaaaggagcaggcatggaagtgttctgagcagcaattttacttaccacacgagtgatggagatgcattgctccttctctgaagggaatcccaaagccttgtgtctacagagaacctggagttcctgaacctgatgctgactagtgaggaaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaagggtgtctgagccctcaaagtgaggggaaaccagggcctgagccaagctagaattccctctctctgactctcaaatcttttagtcattatagatcccccagactttacatgacacagctttatcaccagagagggactgacacccatgtttctctggccccaagggcaaaattcccagggaagtgctctgataggccaagtttgtatcaggtgcccatccctggaaggtgctgttatccatggggaagggatatataagatggaagcttccagtccaatctcatggagaagcagaaatacatatttccaagaagttggatgggtgggtactattctgattacacaaaacaaatgccacacatcacccttaccatgtgcctgatccagcctctcccctgattacaccagcctcgtcttcattaagccctcttccatcatgtccccaaacctgcaagggctccccactgcctactgcatcgagtcaaaactcaaatgcttggcttctcatacgtccaccatggggtcctaccaatagattccccattgcctcctccttcccaaaggactccacccatcctatcagcctgtctcttccatatgacctcatgcatctccacctgctcccaggccagtaagggaaatagaaaaaccctgcccccaaataagaagggatggattccaaccccaactccagtagcttgggacaaatcaagcttcagtttcctggtctgtagaagagggataaggtacctttcacatagagatcatcctttccagcatgaggaactagccaccaactcttgcaggtctcaacccttttgtctgcctcttagacttctgctttccacacctggcactgctgtgctgtgcccaagttgtggtgctgacaaagcttggaagagcctgcaggtgctgctgcgtggcatagcccagacacagaagaggctggttcttacgatggcacccagtgagcactcccaagtctacagagtgatagccttccgtaacccaactctcctggactgccttgaatatcccctcccagtcaccttgtggcaagcccctgcccatctgggaaaataccccatcattcatgctactgccaacctggggagccagggctatgggagcagcttttttttcccccctagaaacgtttggaacaatctaaaagtttaaagctcgaaaacaattgtaataatgctaaagaaaaagtcatccaatctaaccacatcaatattgtcattcctgtattcacccgtccagaccttgttcacactctcacatgtttagagttgcaatcgtaatgtacagatggttttataatctgatttgttttcctcttaacgttagaccacaaatagtgctcgctttctatgtagtttggtaattatcattttagaagactctaccagactgtgtattcattgaagtcagatgtggtaactgttaaattgctgtgtatctgatagctctttggcagtctatatgtttgtataatgaatgagagaataagtcatgttccttcaagatcatgtaccccaatttacttgccattactcaattgataaacatttaacttgtttccaatgtttagcaaatacatattttatagaacttccaaaaaaaaa

SEQ ID NO: 140 - DNA, 3108 bp, Homo sapiens
  sequence of CX3CR1b
gaaatactcgtctctggtaaagtctgagcaggacagggtggctgactggcagatccagaggttcccttggcagtccacgccaggccttcaccatggatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtcctccaggaaatctggcccgtgctccgcaatgtggaaacaaattttcttggcttcctactccccctgctcattatgagttattgctacttcagaatcatccagacgctgttttcctgcaagaaccacaagaaagccaaagccattaaactgatccttctggtggtcatcgtgtttttcctcttctggacaccctacaacgttatgattttcctggagacgcttaagctctatgacttctttcccagttgtgacatgaggaaggatctgaggctggccctcagtgtgactgagacggttgcatttagccattgttgcctgaatcctctcatctatgcatttgctggggagaagttcagaagatacctttaccacctgtatgggaaatgcctggctgtcctgtgtgggcgctcagtccacgttgatttctcctcatctgaatcacaaaggagcaggcatggaagtgttctgagcagcaattttacttaccacacgagtgatggagatgcattgctccttctctgaagggaatcccaaagccttgtgtctacagagaacctggagttcctgaacctgatgctgactagtgaggaaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaagggtgtctgagccctcaaagtgaggggaaaccagggcctgagccaagctagaattccctctctctgactctcaaatcttttagtcattatagatcccccagactttacatgacacagctttatcaccagagagggactgacacccatgtttctctggccccaagggcaaaattcccagggaagtgctctgataggccaagtttgtatcaggtgcccatccctggaaggtgctgttatccatggggaagggatatataagatggaagcttccagtccaatctcatggagaagcagaaatacatatttccaagaagttggatgggtgggtactattctgattacacaaaacaaatgccacacatcacccttaccatgtgcctgatccagcctctcccctgattacaccagcctcgtcttcattaagccctcttccatcatgtccccaaacctgcaagggctccccactgcctactgcatcgagtcaaaactcaaatgcttggcttctcatacgtccaccatggggtcctaccaatagattccccattgcctcctccttcccaaaggactccacccatcctatcagcctgtctcttccatatgacctcatgcatctccacctgctcccaggccagtaagggaaatagaaaaaccctgcccccaaataagaagggatggattccaaccccaactccagtagcttgggacaaatcaagcttcagtttcctggtctgtagaagagggataaggtacctttcacatagagatcatcctttccagcatgaggaactagccaccaactcttgcaggtctcaacccttttgtctgcctcttagacttctgctttccacacctggcactgctgtgctgtgcccaagttgtggtgctgacaaagcttggaagagcctgcaggtgctgctgcgtggcatagcccagacacagaagaggctggttcttacgatggcacccagtgagcactcccaagtctacagagtgatagccttccgtaacccaactctcctggactgccttgaatatcccctcccagtcaccttgtggcaagcccctgcccatctgggaaaataccccatcattcatgctactgccaacctggggagccagggctatgggagcagcttttttttcccccctagaaacgtttggaacaatctaaaagtttaaagctcgaaaacaattgtaataatgctaaagaaaaagtcatccaatctaaccacatcaatattgtcattcctgtattcacccgtccagaccttgttcacactctcacatgtttagagttgcaatcgtaatgtacagatggttttataatctgatttgttttcctcttaacgttagaccacaaatagtgctcgctttctatgtagtttggtaattatcattttagaagactctaccagactgtgtattcattgaagtcagatgtggtaactgttaaattgctgtgtatctgatagctctttggcagtctatatgtttgtataatgaatgagagaataagtcatgttccttcaagatcatgtaccccaatttacttgccattactcaattgataaacatttaacttgtttccaatgtttagcaaatacatattttatagaacttcca

SEQ ID NO: 141 - DNA, 3304 bp, Homo sapiens
  sequence of CX3CL1
ctgagctctgccgcctggctctagccgcctgcctggcccccgccgggactcttgcccaccctcagccatggctccgatatctctgtcgtggctgctccgcttggccaccttctgccatctgactgtcctgctggctggacagcaccacggtgtgacgaaatgcaacatcacgtgcagcaagatgacatcaaagatacctgtagctttgctcatccactatcaacagaaccaggcatcatgcggcaaacgcgcaatcatcttggagacgagacagcacaggctgttctgtgccgacccgaaggagcaatgggtcaaggacgcgatgcagcatctggaccgccaggctgctgccctaactcgaaatggcggcaccttcgagaagcagatcggcgaggtgaagcccaggaccacccctgccgccgggggaatggacgagtctgtggtcctggagcccgaagccacaggcgaaagcagtagcctggagccgactccttcttcccaggaagcacagagggccctggggacctccccagagctgccgacgggcgtgactggttcctcagggaccaggctccccccgacgccaaaggctcaggatggagggcctgtgggcacggagcttttccgagtgcctcccgtctccactgccgccacgtggcagagttctgctccccaccaacctgggcccagcctctgggctgaggcaaagacctctgaggccccgtccacccaggacccctccacccaggcctccactgcgtcctccccagccccagaggagaatgctccgtctgaaggccagcgtgtgtggggtcagggacagagccccaggccagagaactctctggagcgggaggagatgggtcccgtgccagcgcacacggatgccttccaggactgggggcctggcagcatggcccacgtctctgtggtccctgtctcctcagaagggacccccagcagggagccagtggcttcaggcagctggacccctaaggctgaggaacccatccatgccaccatggacccccagaggctgggcgtccttatcactcctgtccctgacgcccaggctgccacccggaggcaggcggtggggctgctggccttccttggcctcctcttctgcctgggggtggccatgttcacctaccagagcctccagggctgccctcgaaagatggcaggagagatggcggagggccttcgctacatcccccggagctgtggtagtaattcatatgtcctggtgcccgtgtgaactcctctggcctgtgtctagttgtttgattcagacagctgcctgggatccctcatcctcatacccacccccacccaagggcctggcctgagctgggatgattggaggggggaggtgggatcctccaggtgcacaagctccaagctcccaggcattccccaggaggccagccttgaccattctccaccttccagggacagagggggtggcctcccaactcaccccagccccaaaactctcctctgctgctggctggttagaggttccctttgacgccatcccagccccaatgaacaattatttattaaatgcccagccccttctgacccatgctgccctgtgagtactacagtcctcccatctcacacatgagcatcaggccaggccctctgcccactccctgcaacctgattgtgtctcttggtcctgctgcagttgccagtcaccccggccacctgcggtgctatctcccccagccccatcctctgtacagagcccacgcccccactggtgacatgtcttttcttgcatgaggctagtgtggtgtttcctggcactgcttccagtgaggctctgcccttggttaggcattgtgggaaggggagataagggtatctggtgactttcctctttggtctacactgtgctgagtctgaaggctgggttctgatcctagttccaccatcaagccaccaacatactcccatctgtgaaaggaaagagggaggtaaggaatacctgtccccctgacaacactcattgacctgaggcccttctctccagcccctggatgcagcctcacagtccttaccagcagagcaccttagacagtccctgccaatggactaacttgtctttggaccctgaggcccagagggcctgcaagggagtgagttgatagcacagaccctgccctgtgggcccccaaatggaaatgggcagagcagagaccatccctgaaggccccgcccaggcttagtcactgagacagcccgggctctgcctcccatcacccgctaagagggagggagggctccagacacatgtccaagaagcccaggaaaggctccaggagcagccacattcctgatgcttcttcagagactcctgcaggcagccaggccacaagacccttgtggtcccaccccacacacgccagattctttcctgaggctgggctcccttcccacctctctcactccttgaaaacactgttctctgccctccaagaccttctccttcacctttgtccccaccgcagacaggaccagggatttccatgatgttttccatgagtcccctgtttgtttctgaaagggacgctacccgggaagggggctgggacatgggaaaggggaagttgtaggcataaagtcaggggttcccttttttggctgctgaaggctcgagcatgcctggatggggctgcaccggctggcctggcccctcagggtccctggtggcagctcacctctcccttggattgtccccgacccttgccgtctacctgaggggcctcttatgggctgggttctacccaggtgctaggaacactccttcacagatgggtgcttggaggaaggaaacccagctctggtccatagagagcaagacgctgtgctgccctgcccacctggcctctgcactcccctgctgggtgtggcgcagcatattcaggaagctcagggcctggctcaggtggggtcactctggcagctcagagagggtgggagtgggtccaatgcactttgttctggctcttccaggctgggagagcctttcaggggtgggacaccctgtgatggggccctgcctcctttgtgaggaagccgctggggccagttggtcccccttccatggactttgttagtttctccaagcaggacatggacaaggatgatctaggaagactttggaaagagtaggaagactttggaaagacttttccaaccctcatcaccaacgtctgtgccattttgtattttactaataaaatttaaaagtcttgtgaaaaaaaaaaaaaaaaaaaaaaaaaaaaaaaaa

(19) United States
(12) Patent Application Publication
(10) Pub. No.: US 2016/0130350 A1
(43) Pub. Date: May 12, 2016

(54) ANTI-CXCL9, ANTI-CXCL10, ANTI-CXCL11, ANTI-CXCL13, ANTI-CXCR3 AND ANTI-CXCR5 AGENTS FOR INHIBITION OF INFLAMMATION

(71) Applicant: JYANT TECHNOLOGIES, INC., Marietta, GA (US)

(72) Inventor: James W. Lillard, JR., Smyrna, GA (US)

(21) Appl. No.: 14/843,811

(22) Filed: Sep. 2, 2015

Related U.S. Application Data
(63) Continuation of application No. 13/535,202, filed on Jun. 27, 2012, now abandoned.
Application No. 13/535,202 is a continuation-in-part of application No. 13/105,406, filed on May 11, 2011, now Pat. No. 8,318,170.
Application No. 13/105,406 is a continuation of application No. 10/712,393, filed on Nov. 14, 2003, now Pat. No. 7,964,194.
(60) Provisional application No. 60/426,350, filed on Nov. 15, 2002.

(51) Int. Cl. C07K 16/28; A61K 45/06; A61K 39/395; C07K 16/24; A61K 31/713

(52) U.S. Cl. CPC C07K 16/2866; C07K 16/24; A61K 31/713; A61K 39/3955; A61K 45/06; C07K 2317/76; C07K 2317/92; C07K 2317/24; A61K 2039/505

(57) ABSTRACT

Methods for preventing or inhibiting inflammation in a subject are disclosed. In one aspect, the method comprises administering to a subject diagnosed with an inflammatory disease an effective amount of an anti-inflammatory agent that (1) inhibits the expression of CXCL9, CXCL10, CXCL11, CXCL13, CXCR3 and/or CXCR5, or (2) inhibits the interaction between CXCR3 and CXCL9, CXCL10 or CXCL11, or between CXCR5 and CXCL13, or (3) inhibits a biological activity of CXCL9, CXCL10, CXCL11, CXCL13, CXCR3 and/or CXCR5, wherein the agent comprises an antibody, antibody fragment, short interfering RNA (siRNA), aptamer, synbody, binding agent, peptide, aptamer-siRNA chimera, single stranded antisense oligonucleotide, triplex forming oligonucleotide, ribozyme, external guide sequence, or an agent-encoding expression vector.

This application is a Continuation of U.S. patent application Ser. No. 13/535,202 filed on Jun. 27, 2012, which is a continuation-in-part of U.S. patent application Ser. No. 13/105,406 filed on May 11, 2011, now U.S. Pat. No. 8,318,170, which is a continuation of U.S. patent application Ser. No. 10/712,393, filed on Nov. 14, 2003, now U.S. Pat. No. 7,964,194, which claims priority to U.S. Provisional Patent Application No. 60/426,350, filed Nov. 15, 2002. The entirety of all of the aforementioned applications is incorporated herein by reference.

### FIELD

This application generally relates to methods and compositions for inhibiting inflammation. In particular, the application relates to the use of anti-CXCL9, anti-CXCL10, anti-CXCL11, anti-CXCL13, anti-CXCR3 and/or anti-CXCR5 agents, and/or other anti-inflammatory agents for prevention and treatment of inflammatory diseases.

### BACKGROUND

Despite recent advances in studies related to the inflammation process, therapies for treatment of chronic inflammatory diseases have remained largely elusive. This is perhaps a result of the many and complex factors in the host that initiate and maintain inflammatory conditions. Current therapies have disadvantages associated with them, including the suppression of the immune system that can render the host more susceptible to bacterial, viral and parasitic infections. For example, use of steroids is a traditional approach to chronic inflammation treatment. Such treatment can lead to changes in weight and suppression of protective immunity. Advances in biotechnology have promoted the development of targeted biologicals with fewer side effects. To improve inflammatory disease treatment, technologies that alter and control the factors generated by cells of both innate and adaptive immunity systems need to be developed.

Host cells have surface receptors that associate with ligands to signal and regulate host cell activities. Administration of anti-TNF-α antibody or soluble TNF-α receptor has been shown to inhibit inflammatory diseases. Unfortunately, the side effects associated with this treatment can result in an increased risk of infections (e.g., tuberculosis) and other adverse reactions by mechanisms not fully understood. Similarly, antibody therapies focused on membrane bound molecules like CD40 have a propensity for inhibiting inflammation and graft-host diseases. While other targeted host cell therapies to prevent inflammatory diseases are being developed, there is no known single surface or secreted factor that will stop all inflammatory diseases. Consequently, the development of therapies to exploit newly identified specific host cell targets is required.

A variety of pathogens or toxins activate macrophages, neutrophils, T cells, B cells, monocytes, NK cells, Paneth and crypt cells, as well as epithelial cells shortly after entry into the mucosa. Chemokines represent a superfamily of small, cytokine-like proteins that are resistant to hydrolysis, promote neovascularization or endothelial cell growth inhibition, induce cytoskeletal rearrangement, activate or inactivate lymphocytes, and mediate chemotaxis through interactions with G-protein-coupled receptors. Chemokines can mediate the growth and migration of host cells that express their receptors. The cellular mechanisms responsible for the function of chemokines are often, but not entirely, Ca²⁺ flux dependent and pertussis toxin-sensitive. However, the precise mechanisms for chemokine-mediated events are not known.

### SUMMARY

The present invention relates to methods and compositions for treating or preventing inflammatory diseases or conditions. In one embodiment, the method comprises the step of administering to a subject diagnosed with an inflammatory disease or condition an effective amount of an anti-inflammatory agent that (1) inhibits the expression of CXCL9, CXCL10, CXCL11, CXCL13, CXCR3 and/or CXCR5, or (2) inhibits the interaction between any one of CXCL9, CXCL10, CXCL11, CXCL13, CXCR3 and/or CXCR5, or (3) inhibits a biological activity of CXCL9, CXCL10, CXCL11, CXCL13, CXCR3 and/or CXCR5.

In another embodiment, the method comprises the step of administering to a subject diagnosed with an inflammatory disease or condition a therapeutically effective amount of an anti-CXCL9 antibody, an anti-CXCL10 antibody, an anti-CXCL11 antibody, an anti-CXCL13 antibody, and anti-CXCR3 antibody, an anti-CXCR5 antibody, or combination thereof.

In one embodiment, the agent or antibody is administered in a dosage range from about 10 μg/kg body weight/day to about 10 mg/kg body weight/day.

The agent may comprise an antibody, antibody fragment, short interfering RNA (siRNA), aptamer, synbody, binding agent, peptide, aptamer-siRNA chimera, single stranded antisense oligonucleotide, triplex forming oligonucleotide, ribozyme, external guide sequence, or agent-encoding expression vector.

In another aspect, a method for enhancing effect of anti-inflammatory therapy, comprises administering to a subject who is receiving or has received anti-inflammatory therapy an effective amount of an anti-inflammatorying that (1) inhibits the expression of CXCL9, CXCL10, CXCL11, CXCL13, CXCR3 and/or CXCR5, or (2) inhibits the interaction between CXCR3 and CXCL9, CXCL10 or CXCL11, and the interaction between CXCR5 and CXCL13, or (3) inhibits a biological activity of CXCL9, CXCL10, CXCL11, CXCL13, CXCR3 and/or CXCR5, wherein the agent comprises an antibody, antibody fragment, short interfering RNA (siRNA), aptamer, synbody, binding agent, peptide, aptamer-siRNA chimera, single stranded antisense oligonucleotide, triplex forming oligonucleotide, ribozyme, external guide sequence, or agent-encoding expression vector.

In one embodiment, the subject is receiving anti-inflammatory therapy. In another embodiment, the subject has received anti-inflammatory therapy and has exhibited anti-inflammatory drug-resistance to an anti-inflammatory agent.

In a further aspect the present invention provides a pharmaceutical composition, comprising an anti-inflammatory agent capable of (1) inhibiting the expression of CXCL9, CXCL10, CXCL11, CXCL13, CXCR3 and/or CXCR5; (2) inhibiting the interaction between any one of CXCL9, CXCL10, CXCL11, CXCL13, CXCR3 and/or CXCR5, or (3) inhibiting a biological activity of CXCL9, CXCL10, CXCL11, CXCL13, CXCR3 and/or CXCR5, wherein the anti-inflammatory agent is an antibody, antibody fragment, short interfering RNA (siRNA), aptamer, synbody, binding agent, peptide, aptamer-siRNA chimera, single stranded antisense oligonucleotide, triplex forming oligonucleotide, ribozyme, external guide sequence, or agent-encoding expression vector; and a pharmaceutically acceptable carrier.

### BRIEF DESCRIPTION OF THE DRAWINGS

FIG. 1 shows IFN-γ, IP-10, MIG, I-TAC, and CXCR3 mRNA expression during murine colitis.

FIG. 2 shows histological analysis of IBD in TCR β×δ^{-/-} mice that received CD45RB^{HI} or CXCR3⁺ CD4⁺ T cells by adoptive transfer.

FIG. 3 shows SAA levels and the development of colitis in IL-10^{-/-} mice. SAA concentrations >200 μg/ml were associated with the onset of asymptomatic colitis at week 0.

FIG. 4 shows changes in body weight of IL-10^{-/-} mice.

FIG. 5 shows association of serum IL-6 and SAA levels with murine colitis.

FIG. 6 shows total fecal and serum Ab levels in IL-10^{-/-} mice.

FIG. 7 shows serum IL-12, IFN-γ, IL-2, TNF-α, IL-1α, and IL-1β levels in IL-10^{-/-} mice with IBD.

FIG. 8 shows histological characteristics of colitis presented by IL-10^{-/-} mice.

FIG. 9 shows that anti-CXCL10 antibody abrogates severe colitis.

FIG. 10 shows Th1 cytokine, CXCL10 and CXCR3 mRNA expression in mucosal tissue during severe colitis.

FIG. 11 shows Th1 and inflammatory cytokine levels in serum during severe colitis progression.

FIG. 12 shows anti-CXCL10 antibody effects on colitis pathology.

FIG. 13 shows histological and immunofluorescence localization of CXCL9, CXCL10, CXCL11, and TNF-α in the colon of CD patients.

FIG. 14 shows M. avium subsp. paratuberculosis (MAP)-specific serum Ab responses in IL-10^{-/-} mice during spontaneous colitis.

FIG. 15 shows histological characteristics of IL-10^{-/-} mice challenged with M. avium subsp. paratuberculosis (MAP).

FIG. 16 shows changes in body weight of IL-10^{-/-} mice after MAP challenge.

FIG. 17 shows serum cytokine levels in IL-10^{-/-} mice after MAP challenge.

FIG. 18 shows anti-peptide #25 Ag (from MPT59)-induced proliferation and IL-2 production by CD4⁺ T cells from IL-10^{-/-} mice.

FIG. 19 shows serum CXCR3 ligands and mycobacterial-specific Ab responses in IBD patients.

FIG. 20 shows changes in SAA levels in IBD patients and in IL-10^{-/-} mice after mycobacterial challenge.

FIG. 21 shows intestinal histological characteristics of IL-10^{-/-} mice challenged with Mycobacteria.

FIG. 22 shows serum CXCL9, CXCL10 and CXCL11 concentrations in IC patients.

FIG. 23 shows histological changes after CYP-induced cystitis.

FIG. 24 shows CXCR3, CXCL9, CXCL10, and CXCL11 mRNA expression in CYP-treated mice.

FIG. 25 shows upregulated CXCL10 expression during active CD.

FIG. 26 shows upregulated expression of CXCL11 and CXCL9 during active CD.

FIG. 27 shows upregulated serum concentrations of serum amyloid A (SAA) and IL-6 in CD patients.

FIG. 28 shows serum IL-12p40 and IFN-γ levels correlate during CD.

FIG. 29 shows inflammatory cytokine levels during active CD.

FIG. 30 shows histological characteristics of colitis in normal and CD patients with high serum CXCR3 ligand concentrations.

FIG. 31 shows CXCR3 ligands and TNFα expression in colons of normal and CD patients by histopathological examination.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the widest possible scope consistent with the principles and features disclosed herein.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

### DEFINITIONS

As used herein, the following terms shall have the following meanings:

The terms “treat,” “treating” or “treatment” as used herein, refers to a method of alleviating or abrogating a disorder and/or its attendant symptoms. The terms “prevent”, “preventing” or “prevention,” as used herein, refer to a method of barring a subject from acquiring a disorder and/or its attendant symptoms. In certain embodiments, the terms “prevent,” “preventing” or “prevention” refer to a method of reducing the risk of acquiring a disorder and/or its attendant symptoms.

As used herein, the terms “anti-inflammatory activity” or “anti-inflammatory response” refer to a reduction or prevention of inflammation manifested in a change in cells, such as proliferation, activation, gene expression, and the like. A reduction in inflammation may include, for example, reducing the secretion or expression of inflammatory cytokines, chemokines, cytokine/chemokine receptors; adhesion molecules, proteases, and/or immunoglobulins; reducing chemotaxis or migration of cells; reducing the blood concentration of monocytes and/or local accumulation thereof at the sites of inflammation; increasing apoptosis of immune cells; suppressing class-II MHC presentation; reducing the number of autoreactive cells; increasing immune tolerance, reducing autoreactive cell survival, combinations thereof, and the like.

As used herein, the term “anti-inflammatory agent” refers to a biologic agent which, upon binding to a protein reduces or prevents inflammatory activity or upon binding to a nucleic acid encoding an inflammatory protein product reduces or blocks expression of an mRNA or protein corresponding to the inflammatory protein product. Anti-inflammatory agents are to be distinguished from anti-inflammatory small molecule chemical compounds as further described herein. Exemplary anti-inflammatory agents include antibodies, antibody fragments, short interfering RNAs (siRNAs), aptamers, synbodies, binding agents, peptides, aptamer-siRNA chimeras, single stranded antisense oligonucleotides, triplex forming oligonucleotides, ribozymes, external guide sequences, agent-encoding expression vectors, and the like.

As used herein, the term “antibody” refers to immunoglobulin molecules and immunologically active portions of immunoglobulin (Ig) molecules, i.e., molecules that contain an antigen binding site or epitope binding domain that specifically binds (immunoreacts with) an antigen. The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit specific binding to a target antigen. By “specifically bind” or “immunoreacts with” is meant that the antibody reacts with one or more antigenic determinants of the desired antigen and does not react (i.e., bind) with other polypeptides or binds at much lower affinity with other polypeptides. The term “antibody” also includes antibody fragments that comprise a portion of a full length antibody, generally the antigen binding or variable region thereof.

The term “anti-inflammatory antibody” refers to an antibody or antibody fragment agent.

The term “monoclonal antibody” as used herein refers to an antibody obtained from a population of substantially homogeneous antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

“Humanized” forms of non-human antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and/or capacity. Methods for making humanized and other chimeric antibodies are known in the art.

“Bispecific antibodies” are antibodies that have binding specificities for at least two different antigens.

The use of “heteroconjugate antibodies” is also within the scope of the present invention. Heteroconjugate antibodies are composed of two covalently joined antibodies. Such antibodies have, for example, been proposed to target immune system cells to unwanted cells. It is contemplated that the antibodies can be prepared in vitro using known methods in synthetic protein chemistry, including those involving the use of crosslinking agents. Alternatively, they may be prepared by fusing two antibodies or fragments thereof by recombinant DNA techniques known to those of skill in the art.

As used herein, the term “nucleic acid” refers to a polydeoxyribonucleotide (DNA or an analog thereof) or polyribonucleotide (RNA or an analog thereof) made up of at least two, and preferably ten or more bases linked by a backbone structure. In DNA, the common bases are adenine (A), guanine (G), thymine (T) and cytosine (C), whereas in RNA, the common bases are A, G, C and uracil (U, in place of T), although nucleic acids may include base analogs (e.g., inosine) and abasic positions (i.e., a phosphodiester backbone that lacks a nucleotide at one or more positions). Exemplary nucleic acids include single-stranded (ss), double-stranded (ds), or triple-stranded polynucleotides or oligonucleotides of DNA and RNA.

The term “polynucleotide” refers to nucleic acids containing more than 10 nucleotides.

The term “oligonucleotide” refers to a single stranded nucleic acid containing between about 15 to about 100 nucleotides.

The term “promoter” is to be taken in its broadest context and includes transcriptional regulatory elements (TREs) from genomic genes or chimeric TREs therefrom, including the TATA box or initiator element for accurate transcription initiation, with or without additional TREs (i.e., upstream activating sequences, transcription factor binding sites, enhancers, and silencers) which regulate activation or repression of genes operably linked thereto in response to developmental and/or external stimuli, and trans-acting regulatory proteins or nucleic acids. The promoter may be constitutively active or it may be active in one or more tissues or cell types in a developmentally regulated manner. A promoter may contain a genomic fragment or it may contain a chimera of one or more TREs combined together.

In a pharmacological sense, in the context of the present invention, a “therapeutically effective amount” of an anti-inflammatory antibody, agent or small molecule inhibitor, or combination thereof refers to an amount effective in the prevention or treatment of a disorder for the treatment of which the anti-inflammatory agent or combination thereof is effective. A “disorder” or “disease” is any inflammatory condition that would benefit from treatment with the antibody, agent or small molecule inhibitor.

The term “inflammatory bowel disease” or “IBD” refers to the group of disorders that cause the intestines to become inflamed, generally manifested with symptoms including abdominal cramps and pain, diarrhea, weight loss and intestinal bleeding. The main forms of IBD are ulcerative colitis (UC) and Crohn's disease.

The term “ulcerative colitis” or “UC” is a chronic, episodic, inflammatory disease of the large intestine and rectum characterized by bloody diarrhea. Ulcerative colitis is characterized by chronic inflammation in the colonic mucosa and can be categorized according to location: “proctitis” involves only the rectum, “proctosigmoiditis” affects the rectum and sigmoid colon, “left-sided colitis” encompasses the entire left side of the large intestine, “pancolitis” inflames the entire colon.

The term “Crohn's disease,” also called “regional enteritis,” is a chronic autoimmune disease that can affect any part of the gastrointestinal tract but most commonly occurs in the ileum (the area where the small and large intestine meet). Crohn's disease, in contrast to ulcerative colitis, is characterized by chronic inflammation extending through all layers of the intestinal wall and involving the mesentery as well as regional lymph nodes. Whether or not the small bowel or colon is involved, the basic pathologic process is the same.

Ulcerative colitis and Crohn's disease can be distinguished from each other clinically, endoscopically, pathologically, and serologically in more than 90% of cases; the remainder are considered to be indeterminate IBD.

The term “mucosal tissue” refers to any tissue in which mucosal cells are found, such tissues, include, for example, gastro-intestinal tissues (e.g., stomach, small intestine, large intestine, rectum), uro-genital tissue (e.g., vaginal tissue, penile tissue, urethra), nasal-larynx tissue (e.g., nasal tissue, larynx tissue), mouth (buccal tissue) to name a few. Other mucosal tissues are known and easily identifiable by one of skill in the art.

The term “inhibits” is a relative term, an agent inhibits a response or condition if the response or condition is quantitatively diminished following administration of the agent, or if it is diminished following administration of the agent, as compared to a reference agent. Similarly, the term “prevents” does not necessarily mean that an agent completely eliminates the response or condition, so long as at least one characteristic of the response or condition is eliminated. Thus, a composition that reduces or prevents an inflammatory response, can, but does not necessarily completely eliminate such a response, so long as the response is measurably diminished, for example, by at least about 50%, such as by at least about 70%, or about 80%, or even by about 90% of (that is to 10% or less than) the response in the absence of the agent, or in comparison to a reference agent.

The term “increased level” refers to a level that is higher than a normal or control level customarily defined or used in the relevant art. For example, an increased level of immunostaining in a tissue is a level of immunostaining that would be considered higher than the level of immunostaining in a control tissue by a person of ordinary skill in the art.

The term “biological sample,” as used herein, refers to material of a biological origin, which may be a body fluid or body product such as blood, plasma, urine, saliva, spinal fluid, stool, sweat or breath. A biological sample may include tissue samples, cell samples, or combination thereof.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

Methods for Inhibiting Inflammation Using Anti-Inflammatory Agents that Inhibit the Expression or Activity of CXCL9, CXCL10, CXCL11, CXCL13, CXCR3 or CXCR5

CXCL9, CXCL10, and CXCL11 chemokines are ligands for the CXCR3 chemokine receptor. CXCL13 chemokine is the ligands for the CXCR5 chemokine receptor. Each of these chemokine ligands and their receptor are locally upregulated and play a role in various inflammatory diseases, including inflammatory bowel diseases. Additionally, CXCL9, -CXCL10, CXCL11 and CXCL13 chemokines enhance inflammation both in vivo and in vitro. CXCR3 and CXCR3 are members of the chemokine receptor family of G protein coupled receptors (GPCRs). Interaction of CXCR3 with CXCL9, CXCL10, and CXCL11 and/or interaction of CXCR5 with CXCL13 activate inflammation.

One aspect of the present application relates to methods for inhibiting inflammation using agents that inhibit the expression or activity of CXCL9, CXCL10, CXCL11 CXCL13, CXCR3 or CXCR5. “Activities” include, for example, transcription, translation, intracellular translocation, secretion, signal transduction, phosphorylation by kinases, cleavage by proteases, homophilic and heterophilic binding to other proteins, ubiquitination, and the like.

In some embodiments, a method for treating or preventing an inflammatory condition in a subject comprises administering to a subject diagnosed with an inflammatory disease an effective amount of an anti-inflammatory agent that: (1) inhibits the expression of CXCL9, CXCL10, CXCL11, CXCL13, CXCR3 and/or CXCR5, or (2) inhibits the interaction between CXCR3 and any one of CXCL9, CXCL10, and CXCL11 or interaction between CXCR5 and CXCL13, or (3) inhibits a biological activity of CXCL9, CXCL10, CXCL11, CXCL13, CXCR3 and/or CXCR5.

In certain embodiments, a therapeutically effective amount of at least one anti-CXCL9, anti-CXCL10, anti-CXCL11, anti-CXCL13, anti-CXCR3 and/or anti-CXCR5 antibody is administered to a subject in need thereof as a sole anti-inflammatory agent. In other embodiments, a therapeutically effective amount of at least one anti-CXCL9, anti-CXCL10, anti-CXCL11, anti-CXCL13, anti-CXCR3 and/or anti-CXCR5 antibody is administered to a subject in need thereof as a primary anti-inflammatory agent, in conjunction with the treatment of the subject beforehand, at the same time, or afterward with a therapeutically effective amount of a secondary anti-inflammatory agent.

An anti-inflammatory agent is a biologic agent capable of reducing or preventing inflammation. Exemplary anti-inflammatory agent include anti-inflammatory antibodies, short interfering RNAs (siRNAs), CXCL9-binding agents, CXCL10-binding agents, CXCL11-binding agents, CXCL13-binding agents, CXCR5-binding agents and CXCR3-binding agents, antisense oligonucleotides, ribozymes, triplex forming oligonucleotides, external guide sequences, agent-encoding expression vectors and anti-inflammatory small molecule chemical compounds.

In a preferred embodiment, the method comprises administering to a subject diagnosed with an inflammatory disease a therapeutically effective amount of an anti-CXCL9 antibody, an anti-CXCL10 antibody, an anti-CXCL11 antibody, an anti-CXCR3 antibody, an anti-CXCL13 antibody, an anti-CXCR5 antibody or a combination thereof, resulting in reduced inflammation.

Exemplary inflammatory diseases or conditions include, but are not limited to, anaphylaxis, septic shock, osteoarthritis, rheumatoid arthritis, psoriasis, asthma, allergies (e.g., drug, insect, plant, food), atherosclerosis, delayed type hypersensitivity, dermatitis, diabetes mellitus, juvenile onset diabetes, graft rejection, inflammatory bowel diseases, such as Crohn's disease, ulcerative colitis, enteritis, and interstitial cystitis; multiple sclerosis, myasthemia gravis, Grave's disease, Hashimoto's thyroiditis, pneumonitis, prostatitis, psoriasis, nephritis, pneumonitis, chronic obstructive pulmonary disease, chronic bronchitis rhinitis, spondyloarthropathies, scheroderma, systemic lupus erythematosus, and thyroiditis. In a preferred embodiment, the inflammatory condition is an inflammatory bowel disease selected from the group consisting of Crohn's disease, ulcerative colitis, enteritis, and interstitial cystitis (including drug-induced cystitis and spontaneous cystitis).

In some embodiments, the subject is diagnosed with an inflammatory condition that results in elevated CXCL9, CXCL10, CXCL11, CXCL13, CXCR3 and/or CXCR5 expression. In other embodiments, the method of treatment further comprises the step of determining whether the level of CXCL9, CXCL10, CXCL11, CXCL13, CXCR3 and/or CXCR53 expression is elevated in a tissue from the subject, and, if so, administering to the subject a therapeutically effective amount of an anti-inflammatory agent that: (1) inhibits the expression of CXCL9, CXCL10, CXCL11, CXCL13, CXCR3 and/or CXCR5, or (2) inhibits the interaction between CXCR3 and any one of CXCL9, CXCL10, and CXCL11, or the interation between CXCR5 and CXCL13, or (3) inhibits a biological activity of CXCL9, CXCL10, CXCL11, CXCL13, CXCR3 and/or CXCR5.

In some embodiments, the therapeutically effective amount of an anti-CXCL9, anti-CXCL10, anti-CXCL11, anti-CXCL13, anti-CXCR3 and/or anti-CXCR5 anti-inflammatory agent augments the effectiveness of one or more additional therapeutically effective agents or small molecule agents in inhibiting inflammation. In a more particular embodiment, the therapeutically effective amount of the anti-CXCL9, anti-CXCL10, anti-CXCL11, anti-CXCL13, anti-CXCR3 and/or anti-CXCR5 anti-inflammatory agent reduces the amount of the one or more additional therapeutically effective agents or small molecule agents required for inhibiting inflammation.

In particular embodiments, treatment of a subject with an anti-CXCL9, anti-CXCL10, anti-CXCL11, anti-CXCL13, anti-CXCR5 and/or anti-CXCR3 anti-inflammatory agent is carried out in conjunction with the treatment of the subject beforehand, at the same time, or afterward with a therapeutically effective amount of at least one secondary agent directed against a chemokine, cytokine, receptor thereof, or derivatives thereof, including soluble receptors and the like.

In one embodiment, a method for enhancing effect of anti-inflammatory therapy comprises administering to a subject who is receiving or has received anti-inflammatory therapy an effective amount of an anti-inflammatory agent that (1) inhibits the expression of CXCL9, CXCL10, CXCL11, CXCL13, CXCR3 and/or CXCR5, or (2) inhibits the interaction between any one of CXCL9, CXCL10, CXCL11, CXCL13, CXCR3 and/or CXCR5 or (3) inhibits a biological activity of CXCL9, CXCL10, CXCL11, CXCL13, CXCR3 and/or CXCR5, wherein the agent comprises an antibody, antibody fragment, short interfering RNA (siRNA), aptamer, synbody, binding agent, peptide, aptamer-siRNA chimera, single stranded antisense oligonucleotide, triplex forming oligonucleotide, ribozyme, external guide sequence, or agent-encoding expression vector.

In a particular embodiment, the subject is receiving anti-inflammatory therapy. In another embodiment, the subject has received anti-inflammatory therapy, but has exhibited anti-inflammatory drug-resistance to an anti-inflammatory agent.

In a preferred embodiment, the subject is administered an effective amount of an anti-CXCL9 antibody, an anti-CXCL10 antibody, an anti-CXCL11 antibody, an anti-CXCL13 antibody, anti-CXCR3 antibody, an anti-CXCR5 antibody, or combination thereof for.

An anti-inflammatory agent may include any inhibitor of CXCL9, CXCL10, CXCL11, CXCL13, CXCR3 and/or CXCR5 activity and/or expression. Exemplary anti-inflammatory agents include antibodies, short interfering RNA (siRNA), aptamer-siRNA chimeras, single stranded antisense oligonucleotides, triplex forming oligonucleotides, ribozymes, external guide sequences, agent-encoding expression vectors, and combination thereof.

### Anti-Inflammatory Antibodies

An anti-inflammatory antibody may be an anti-chemokine antibody, an anti-chemokine receptor antibody, anti-cytokine antibody, an anti-cytokine receptor antibody, an anti-proinflammatory peptide antibody, or a combination thereof (e.g., bispecific antibody).

A preferred anti-inflammatory antibody of the present application is one which binds to human CXCL9, CXCL10, CXCL11 or CXCL13 and preferably blocks (partially or completely) the ability of CXCL9, CXCL10, CXCL11 or CXCL13 to bind and/or activate the CXCR3 or CXCR5 receptor. Another preferred antibody of the present invention is one which binds to human CXCR3 or CXCR5 and preferably blocks (partially or completely) the ability of a cell carrying the receptor, such as an epithelial, endothelial or lymphoid cell, from binding to and/or being activated by CXCL9, CXCL10 CXCL11 and/or CXCL13.

In one embodiment, the anti-CXCL9, anti-CXCL10, anti-CXCL11, anti-CXCL13, anti-CXCR3 and/or anti-CXCR5 antibody is a monoclonal antibody. In another embodiment, the anti-CXCL9, anti-CXCL10, anti-CXCL11, anti-CXCL13, anti-CXCR3 and/or anti-CXCR5 antibody is a humanized antibody. In another embodiment, the anti-CXCL9, anti-CXCL10, anti-CXCL11, anti-CXCL13, anti-CXCR3 and/or anti-CXCR5 antibody is an antibody fragment. In yet another embodiment, the anti-CXCL9, anti-CXCL10, anti-CXCL11, anti-CXCL13, anti-CXCR3 and/or anti-CXCR5 antibody is a humanized antibody fragment.

In other embodiments, the anti-CXCL9, anti-CXCL10, anti-CXCL11, anti-CXCL13, anti-CXCR3 or anti-CXCR5 antibody binds to CXCL9, CXCL10, CXCL11, CXCL13, CXCR3 or CXCR5, respectively, with a kd value in the range of 0.01 pM to 10 μM, 0.01 pM to 1 μM, 0.01 pM to 100 nM, 0.01 pM to 10 nM, 0.01 pM to 1 nM, 0.1 pM to 10 μM, 0.1 pM to 1 μM, 0.1 pM to 100 nM, 0.1 pM to 10 nM, 0.1 pM to 1 nM, 1 pM to 10 μM, 1 pM to 1 μM, 1 pM to 100 nM, 1 pM to 10 nM, 1 pM to 1 nM, 10 pM to 10 μM, 10 pM to 1 μM, 10 pM to 100 nM, 10 pM to 10 nM, 10 pM to 1 nM, 100 pM to 10 μM, 100 pM to 1 μM and 100 pM to 100 nM. In some other embodiments, the anti-CXCL9, anti-CXCL10, anti-CXCL11, anti-CXCL13, anti-CXCR3 or anti-CXCR5 antibody binds to non-target proteins with a kd value of greater than 100 nM. In certain embodiments, the anti-CXCL9, anti-CXCL10, anti-CXCL11, anti-CXCL13, anti-CXCR3 or anti-CXCR5 antibody binds to the target protein (i.e., CXCL9, CXCL10, CXCL11, CXCL13, CXCR3 or CXCR5, respectively, with a Kd value in the range of 0.01 pM to 100 nM or 0.01 pM to 10 nM, and binds to non-target proteins with a Kd value of greater than 100 nM.

An anti-inflammatory antibody may be administered in any form suitable for neutralizing CXCL9, CXCL10, CXCL11, CXCL13, CXCR3 and/or CXCR5 activity. Exemplary antibody or antibody derived fragments may include any member of the group consisting of: IgG, antibody variable region; isolated CDR region; single chain Fv molecule (scFv) comprising a VH and VL domain linked by a peptide linker allowing for association between the two domains to form an antigen binding site; bispecific scFv dimer; minibody comprising a scFv joined to a CH3 domain; diabody (dAb) fragment; single chain dAb fragment consisting of a VH or a VL domain; Fab fragment consisting of VL, VH, CL and CH1 domains; Fab′ fragment, which differs from a Fab fragment by the addition of a few residues at the carboxyl terminus of the heavy chain CH1 domain, including one or more cysteines from the antibody hinge region; Fab′-SH fragment, a Fab′ fragment in which the cysteine residue(s) of the constant domains bear a free thiol group; F(ab)₂, bivalent fragment comprising two linked Fab fragments; Fd fragment consisting of VH and CH1 domains; derivatives thereof; and any other antibody fragment(s) retaining antigen-binding function. Fv, scFv, or diabody molecules may be stabilized by the incorporation of disulphide bridges linking the VH and VL domains. When using antibody-derived fragments, any or all of the targeting domains therein and/or Fc regions may be “humanized” using methodologies well known to those of skill in the art. In some embodiments, the anti-inflammatory antibody is modified to remove the Fc region.

In particular embodiments, an anti-CXCR3 antibody or antibody fragment thereof is conjugated to or fused to a second antibody or antibody binding fragment to enhance its binding to target cells carrying the CXCR3 receptor.

In addition, an anti-inflammatory agent may be conjugated to one or more secondary anti-inflammatory agent(s), such as an anti-inflammatory small molecule(s) to provide a further level of anti-inflammatory activity.

Short Interfering RNAs (siRNAs).

An siRNA is a double-stranded RNA that can be engineered to induce sequence-specific post-transcriptional gene silencing of mRNAs corresponding to any one of the above-described chemokine, cytokine or receptors thereof.

siRNAs exploit the mechanism of RNA interference (RNAi) for the purpose of “silencing” gene expression of targeted chemokine-, cytokine- or receptor genes. This “silencing” was originally observed in the context of transfecting double stranded RNA (dsRNA) into cells. Upon entry therein, the dsRNA was found to be cleaved by an RNase III-like enzyme, Dicer, into double stranded small interfering RNAs (siRNAs) 21-23 nucleotides in length containing 2 nucleotide overhangs on their 3′ ends. In an ATP dependent step, the siRNAs become integrated into a multi-subunit RNAi induced silencing complex (RISC) which presents a signal for AGO2-mediated cleavage of the complementary mRNA sequence, which then leads to its subsequent degradation by cellular exonucleases.

In one embodiment, the anti-inflammatory agent comprises a synthetic siRNA. Synthetically produced siRNAs structurally mimic the types of siRNAs normally processed in cells by the enzyme Dicer. Synthetically produced siRNAs may incorporate any chemical modifications to the RNA structure that are known to enhance siRNA stability and functionality. For example, in some cases, the siRNAs may be synthesized as a locked nucleic acid (LNA)-modified siRNA. An LNA is a nucleotide analogue that contains a methylene bridge connecting the 2′-oxygen of the ribose with the 4′ carbon. The bicyclic structure locks the furanose ring of the LNA molecule in a 3′-endo conformation, thereby structurally mimicking the standard RNA monomers.

In other embodiments, the anti-inflammatory agent may comprise an expression vector engineered to transcribe a short double-stranded hairpin-like RNA (shRNA) that is processed into a targeted siRNA inside the cell. The shRNAs can be cloned in suitable expression vectors using kits, such as Ambion's SILENCER® siRNA Construction Kit, Imgenex's GENESUPPRESSOR™ Construction Kits, and Invitrogen's BLOCK-IT™ inducible RNAi plasmid and lentivirus vectors.

Synthetic siRNAs and shRNAs may be designed using well known algorithms and synthesized using a conventional DNA/RNA synthesizer. A variety of chemokine-, cytokine- and receptor-targeted siRNAs may be commercially obtained from Origen (Rockville, Md.).

### CXCL9-, CXCL10-, CXCL11-, CXCL13-, CXCR3- and CXCR5-Binding Agents

In some embodiments, the anti-inflammatory agent is a CXCL9-, CXCL10-, CXCL11-, CXCL13-, CXCR3- or CXCR5-binding agent. The binding agent may comprise any non-antibody protein, peptide, or synthetic binding molecule, such as an aptamer or synbody, which is capable of specifically binding directly or indirectly to CXCL9, CXCL10, CXCL11, CXCL13, CXCR3 or CXCR5 so as to inhibit the interaction and/or activation between CXCR3 and CXCL9, CXCL10 or CXCL11; or the interaction and/or activation between CXCR5 and CXCL13, or which inhibits a biological activity of CXCL9, CXCL10, CXCL11, CXCL13, CXCR3 and/or CXCR5, which is associated with reducing or preventing an inflammatory response.

The CXCL9-, CXCL10-, CXCL11-, CXCL13, CXCR3 and/or CXCR5-binding agents may be produced by any conventional method for generating high-affinity binding ligands, including SELEX, phage display, and other methodologies, including combinatorial chemistry- and/or high throughput methods known to those of skill in the art.

An aptamer is a nucleic acid version of an antibody that comprises a class of oligonucleotides that can form specific three dimensional structures exhibiting high affinity binding to a wide variety of cell surface molecules, proteins, and/or macromolecular structures. Aptamers are commonly identified by an in vitro method of selection sometimes referred to as Systematic Evolution of Ligands by EXponential enrichment or “SELEX”. SELEX typically begins with a very large pool of randomized polynucleotides which is generally narrowed to one aptamer ligand per molecular target. Typically, aptamers are small nucleic acids ranging from 15-50 bases in length that fold into defined secondary and tertiary structures, such as stem-loops or G-quartets.

An aptamer can be chemically linked or conjugated to the above described nucleic acid inhibitors to form targeted nucleic acid inhibitors, such as aptamer-siRNA chimeras. An aptamer-siRNA chimera contains a targeting moiety in the form of an aptamer which is linked to an siRNA. When using an aptamer-siRNA chimera, it is preferable to use a cell internalizing aptamer. Upon binding to specific cell surface molecules, the aptamer can facilitate internalization into the cell where the nucleic acid inhibitor acts. In one embodiment both the aptamer and the siRNA comprises RNA. The aptamer and the siRNA may comprise any nucleotide modifications as further described herein. Preferably, the aptamer comprises a targeting moiety specifically directed to binding cells expressing the chemokine-, cytokine- and/or receptor target genes, such as lymphoid, epithelial cell, and/or endothelial cells.

Synbodies are synthetic antibodies produced from libraries comprised of strings of random peptides screened for binding to target proteins of interest. Synbodies are described in US 2011/0143953 and Diehnelt et al., PLoS One, 5(5):e10728 (2010).

CXCL9-, CXCL10-, CXCL11-, CXCL13-, CXCR3-, CXCR5-binding agents, including aptamers and synbodies, can be engineered to bind target molecules very tightly with Kds between 10⁻¹⁰ to 10⁻¹² M. In some embodiments, the CXCL9-, CXCL10-, CXCL11-, CXCL13-, CXCR3- or CXCR5-binding agent bind the target molecule with a Kd less than 10⁻⁶, less than 10⁻⁸, less than 10⁻⁹, less than 10⁻¹⁰, or less than 10⁻¹² M.

### Antisense Oligonucleotides.

In another embodiment, the anti-inflammatory inhibitor agent may comprise an antisense oligonucleotide or polynucleotide capable of inhibiting the expression of CXCL9, CXCL10, CXCL11, CXCL13, CXCR3 and/or CXCR5. The antisense oligonucleotide or polynucleotide may comprise a DNA backbone, RNA backbone, or chemical derivative thereof. In one embodiment, the antisense oligonucleotide or polynucleotide comprises a single stranded antisense oligonucleotide or polynucleotide targeting for degradation. In preferred embodiments, the anti-inflammatory inhibitor agent comprises a single stranded antisense oligonucleotide complementary to a CXCL9, CXCL10, CXCL11, CXCL12, CXCR3 or CXCR5 mRNA sequence. The single stranded antisense oligonucleotide or polynucleotide may be synthetically produced or it may be expressed from a suitable expression vector. The antisense nucleic acid is designed to bind via complementary binding to the mRNA sense strand so as to promote RNase H activity, which leads to degradation of the mRNA. Preferably, the antisense oligonucleotide is chemically or structurally modified to promote nuclease stability and/or increased binding.

In some embodiments, the antisense oligonucleotides are modified to produce oligonucleotides with nonconventional chemical or backbone additions or substitutions, including but not limited to peptide nucleic acids (PNAs), locked nucleic acids (LNAs), morpholino backboned nucleic acids, methylphosphonates, duplex stabilizing stilbene or pyrenyl caps, phosphorothioates, phosphoroamidates, phosphotriesters, and the like. By way of example, the modified oligonucleotides may incorporate or substitute one or more of the naturally occurring nucleotides with an analog; internucleotide modifications incorporating, for example, uncharged linkages (e.g., methyl phosphonates, phosphotriesters, phosphoamidates, carbamates, etc.) or charged linkages (e.g., phosphorothioates, phosphorodithioates, etc.); modifications incorporating intercalators (e.g., acridine, psoralen, etc.), chelators (e.g., metals, radioactive metals, boron, oxidative metals, etc.), or alkylators, and/or modified linkages (e.g., alpha anomeric nucleic acids, etc.).

In some embodiments, the single stranded oligonucleotides are internally modified to include at least one neutral charge in its backbone. For example, the oligonucleotide may include a methylphosphonate backbone or peptide nucleic acid (PNA) complementary to the target-specific sequence. These modifications have been found to prevent or reduce helicase-mediated unwinding. The use of uncharged probes may further increase the rate of hybridization to polynucleotide targets in a sample by alleviating the repulsion of negatively-charges nucleic acid strands in classical hybridization.

PNA oligonucleotides are uncharged nucleic acid analogs for which the phosphodiester backbone has been replaced by a polyamide, which makes PNAs a polymer of 2-aminoethyl-glycine units bound together by an amide linkage. PNAs are synthesized using the same Boc or Fmoc chemistry as are use in standard peptide synthesis. Bases (adenine, guanine, cytosine and thymine) are linked to the backbone by a methylene carboxyl linkage. Thus, PNAs are acyclic, achiral, and neutral. Other properties of PNAs are increased specificity and melting temperature as compared to nucleic acids, capacity to form triple helices, stability at acid pH, non-recognition by cellular enzymes like nucleases, polymerases, etc.

Methylphosphonate-containing oligonucleotides are neutral DNA analogs containing a methyl group in place of one of the non-bonding phosphoryl oxygens. Oligonucleotides with methylphosphonate linkages were among the first reported to inhibit protein synthesis via anti-sense blockade of translation.

In some embodiments, the phosphate backbone in the oligonucleotides may contain phosphorothioate linkages or phosphoroamidates. Combinations of such oligonucleotide linkages are also within the scope of the present invention.

In other embodiments, the oligonucleotide may contain a backbone of modified sugars joined by phosphodiester internucleotide linkages. The modified sugars may include furanose analogs, including but not limited to 2-deoxyribofuranosides, α-D-arabinofuranosides, α-2′-deoxyribofuranosides, and 2′,3′-dideoxy-3′-aminoribofuranosides. In alternative embodiments, the 2-deoxy-β-D-ribofuranose groups may be replaced with other sugars, for example, β-D-ribofuranose. In addition, β-D-ribofuranose may be present wherein the 2-OH of the ribose moiety is alkylated with a C1-6 alkyl group (2-(O—C1-6 alkyl) ribose) or with a C2-6 alkenyl group (2-(O—C2-6 alkenyl) ribose), or is replaced by a fluoro group (2-fluororibose).

Related oligomer-forming sugars include those used in locked nucleic acids (LNA) as described above. Exemplary LNA oligonucleotides include modified bicyclic monomeric units with a 2′-O-4′-C methylene bridge, such as those described in U.S. Pat. No. 6,268,490, the disclosures of which are incorporated by reference herein.

Chemically modified oligonucleotides may also include, singly or in any combination, 2′-position sugar modifications, 5-position pyrimidine modifications (e.g, 5-(N-benzylcarboxyamide)-2′-deoxyuridine, 5-(N-isobutylcarboxyamide)-2′-deoxyuridine, 5-(N-[2-(1H-indole-3yl)ethyl]carboxyamide)-2′-deoxyuridine, 5-(N-[1-(3-trimethylammonium) propyl]carboxyamide)-2′-deoxyuridine chloride, 5-(N-napthylcarboxyamide)-2′-deoxyuridine, and 5-(N-[1-(2,3-dihydroxypropyl)]carboxyamide)-2′-deoxyuridine), 8-position purine modifications, modifications at exocyclic amines, substitution of 4-thiouridine, substitution of 5-bromo- or 5-iodo-uracil, methylations, unusual base-pairing combinations, such as the isobases isocytidine and isoguanidine, and the like.

### Ribozymes

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, hairpin ribozymes, and tetrahymena ribozymes. There are also a number of ribozymes that are not found in natural systems, but which have been engineered to catalyze specific reactions de novo. Preferred ribozymes cleave RNA or DNA substrates, and more preferably cleave RNA substrates, such as CXCL9, CXCL10, CXCL11, CXCL13, CXCR3 or CXCR5 mRNAs. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

### Triplex Forming Oligonucleotides (TFOs)

Triplex forming oligonucleotides (TFOs) are molecules that can interact with either double-stranded and/or single-stranded nucleic acids, including CXCL9, CXCL10, CXCL11, CXCL13, CXCR3 or CXCR5 genomic DNA regions or their corresponding mRNAs. When TFOs interact with a target region, a structure called a triplex is formed, in which there are three strands of DNA forming a complex dependant on both Watson-Crick and Hoogsteen base-pairing. TFOs can bind target regions with high affinity and specificity. In preferred embodiments, the triplex forming molecules bind the target molecule with a Kd less than 10⁻⁶, 10⁻⁸, 10⁻¹⁰, or 10⁻¹². Exemplary TFOs for use in the present invention include PNAs, LNAs, and LNA modified PNAs, such as Zorro-LNAs.

### External Guide Sequences (EGSs)

External guide sequences (EGSs) are molecules that bind a target nucleic acid molecule forming a complex, and this complex is recognized by RNase P, which cleaves the target molecule. EGSs can be designed to specifically target an mRNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells.

### Agent-Encoding Expression Vectors

In one embodiment, a method for treating or preventing an inflammatory condition in a subject, comprises administering to a subject diagnosed with an inflammatory disease an effective amount of an expression vector expressing an anti-CXCL9, anti-CXCL10, anti-CXCL11, anti-CXCL13 agent, anti-CXCR3 agent and/or anti-CXCR5 agent. In a particular embodiment, the method comprises administering to a subject diagnosed with an inflammatory disease an effective amount of an expression vector expressing an anti-CXCL9, anti-CXCL10, anti-CXCL11, anti-CXCL13, anti-CXCR3 and/or anti-CXCR5 antibody. In another embodiment, the method comprises administering to a subject diagnosed with an inflammatory disease an effective amount of an expression vector expressing an an anti-CXCL9, anti-CXCL10, anti-CXCL11, anti-CXCL13, anti-CXCR3 and/or anti-CXCR5 siRNA. The expression vector can be any expression vector capable of delivering and expressing a polynucleotide encoding an anti-CXCL9, anti-CXCL10, anti-CXCL11, anti-CXCL13, anti-CXCR5 and/or anti-CXCR3 agent including antibodies, siRNAs, antisense oligonucleotides or polynucleotides, and the like.

As used herein, the term “expression vector” includes any nucleic acid capable of directing expression of a nucleic acid. Expression vectors may be delivered to cells using two primary delivery schemes: viral-based delivery systems using viral vectors and non-viral based delivery systems using, for example, plasmid vectors. Such methods are well known in the art and readily adaptable for use with the compositions and methods described herein. In certain cases, these methods can be used to target certain diseases and cell populations by using the targeting characteristics inherent to the carrier or engineered into the carrier.

The nucleic acids that are delivered to cells contain one or more transcriptional regulatory elements, including promoters and/or enhancers, for directing the expression of siRNAs. A promoter comprises a DNA sequence that functions to initiate transcription from a relatively fixed location in regard to the transcription start site. A promoter contains TRE elements required for basic interaction of RNA polymerase and transcription factors, and may operate in conjunction with other upstream elements and response elements. Preferred promoters are those capable of directing expression in a target cell of interest. The promoters may include constitutive promoters (e.g., HCMV, SV40, elongation factor-1α (EF-1α)) or those exhibiting preferential expression in a particular cell type of interest. Enhancers generally refer to DNA sequences that function away from the transcription start site and can be either 5′ or 3′ to the transcription unit. Furthermore, enhancers can be within an intron as well as within the coding sequence. They are usually between 10 and 300 bp in length, and they function in cis. Enhancers function to increase and/or regulate transcription from nearby promoters.

The promotor and/or enhancer may be specifically activated either by light or specific chemical inducing agents. In some embodiments, inducible expression systems regulated by administration of tetracycline or dexamethasone, for example, may be used. In other embodiments, gene expression may be enhanced by exposure to radiation, including gamma irradiation and external beam radiotherapy (EBRT), or alkylating chemotherapeutic drugs.

Cell or tissue-specific transcriptional regulatory elements (TREs) can be incorporated into expression vectors to allow for transcriptional targeting of expression to desired cell types. Expression vectors generally contain sequences for transcriptional termination, and may additionally contain one or more elements positively affecting mRNA stability. An expression vector may further include an internal ribosome entry site (IRES) between adjacent protein coding regions to facilitate expression two or more proteins from a common mRNA in an infected or transfected cell. Additionally, the expression vectors may further include nucleic acid sequence encoding a marker product. This marker product may be used to determine if the gene has been delivered to the cell and is being expressed. Preferred marker genes are the E. coli lacZ gene, which encodes β-galactosidase, and green fluorescent protein (GFP).

Viral-Based Expression Vectors.

In some embodiments, the anti-CXCL9, anti-CXCL10, anti-CXCL11, anti-CXCL13, anti-CXCR3 and/or anti-CXCR5-antibody- or siRNA encoding sequences (or shRNAs) are delivered from viral-derived expression vectors. Exemplary viral vectors may include or be derived from adenovirus, adeno-associated virus, herpesvirus, vaccinia virus, poliovirus, poxvirus, HIV virus, lentivirus, retrovirus, Sindbis and other RNA viruses, and the like. Also preferred are any viral families which share the properties of these viruses which make them suitable for use as vectors. Retroviruses include Murine Moloney Leukemia virus (MMLV), HIV and other lentivirus vectors. Adenovirus vectors are relatively stable and easy to work with, have high titers, and can be delivered in aerosol formulation, and can transfect non-dividing cells. Poxviral vectors are large and have several sites for inserting genes, they are thermostable and can be stored at room temperature. Viral delivery systems typically utilize viral vectors having one or more genes removed and with and an exogenous gene and/or gene/promotor cassette being inserted into the viral genome in place of the removed viral DNA. The necessary functions of the removed gene(s) may be supplied by cell lines which have been engineered to express the gene products of the early genes in trans.

Non-Viral Expression Vectors.

In other embodiments, nonviral delivery systems are utilized for delivery of plasmid vectors or other bioactive non nucleic acid agents using lipid formulations comprising, for example, liposomes, such as cationic liposomes (e.g., DOTMA, DOPE, DC-cholesterol) and anionic liposomes. Liposomes can be further conjugated to one or more proteins or peptides to facilitate targeting to a particular cell, if desired. Administration of a composition comprising a compound and a cationic liposome can be administered to the blood afferent to a target organ or inhaled into the respiratory tract to target cells of the respiratory tract. Furthermore, an anti-inflammatory agent can be administered as a component of a microcapsule or nanoparticle that can be targeted to a cell type of interest using targeting moieties described herein or that can be designed for slow release of one or more anti-inflammatory agent (s) in accordance with a predetermined rate of release or dosage.

In other embodiments, the nucleic acids may be delivered in vivo by electroporation, the technology for which is available from Genetronics, Inc. (San Diego, Calif.) as well as by means of a SONOPORATION machine (ImaRx Pharmaceutical Corp., Tucson, Ariz.). The nucleic acids may be in solution or suspension (for example, incorporated into microparticles, liposomes, or cells). These may be targeted to a particular cell type via antibodies, receptors, or receptor ligands. Vehicles such as “stealth” and other antibody conjugated liposomes (including lipid mediated drug targeting to cells of interest), receptor mediated targeting of DNA through cell specific ligands or viral vectors targeting e.g., lymphoid, epithelial or endothelial cells. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor level regulation. Many receptors follow more than one intracellular pathway, depending on the cell type, receptor concentration, type of ligand, ligand valency, and ligand concentration.

### Secondary Anti-Inflammatory Agents

In some embodiments, a therapeutically effective amount of at least one anti-CXCL9, anti-CXCL10, anti-CXCL11, and/or anti-CXCR3 antibody is administered to a subject in need thereof in conjunction with a secondary anti-inflammatory agent. The a secondary anti-inflammatory agent may be given before, at the same time, or after the administration of the antibody or antibodies. Preferably, the secondary anti-inflammatory agent is directed against a chemokine, cytokine, receptor thereof, or combination thereof.

The secondary anti-inflammatory agent may comprise an anti-inflammatory antibody, short interfering RNA (siRNA), chemokine and chemokine receptor binding agents, antisense oligonucleotides, triplex forming oligonucleotides, ribozymes, external guide sequences, agent-encoding expression vectors or an anti-inflammatory small molecule chemical compound. In some embodiments, the secondary anti-inflammatory agent comprise another an anti-inflammatory antibody directed to determinants on CXCL9, CXCL10, CXCL11, CXCL13, CXCR3 and/or CXCR5. In other embodiments, the secondary anti-inflammatory agent comprises an antibody or an agent directed against a secondary chemokine, cytokine, or receptor thereof.

In some embodiments, the secondary anti-inflammatory agent is an anti-inflammatory agent directed against a chemokine, cytokine or receptor thereof. Exemplary chemokine or chemokine receptor targeted in accordance with the present invention, including protein and cDNA sequences, respectively, from NIH-NCBI GenBank, are described in Table 1.

TABLE 1

| | Protein | | cDNA | |
|---|---|---|---|---|
| Chemokine/ | Accession | SEQ ID | Accession | SEQ ID |
| Receptor | No. | NO: | NO: | NO: |
| CXCL9 | NP_002407 | 1 | NM_002416 | 72 |
| CXCL10 | NP_001556 | 2 | NM_001565 | 73 |
| CXCL11 | NP_005400 | 3 | NM_005409 | 74 |
| CXCL12 | NP_000600 | 4 | NM_000609 | 75 |
| CXCL13 | NP_006410 | 5 | NM_006419 | 76 |
| CXCR3-1 | NP_001495 | 6 | NM_001504 | 77 |
| CXCR3-2 | NP_001136269 | 7 | NM_001142797 | 78 |
| CXCR5-1 | NP_001707 | 8 | NM_001716 | 79 |
| CXCR5-2 | NP_116743 | 9 | NM_032966 | 80 |
| CXCL1 | NP_001502 | 10 | NM_001511 | 81 |
| CXCL2 | NP_002080 | 11 | NM_002089 | 82 |
| CXCL3 | NP_002081 | 12 | NM_002090 | 83 |
| CXCL4 | NP_002610 | 13 | NM_002619 | 84 |
| CXCL5 | NP_002985 | 14 | NM_002994 | 85 |
| CXCL6 | NP_002984 | 15 | NM_002993 | 86 |
| CXCL7 | NP_002695 | 16 | NM_002704 | 87 |
| CXCL8 | NP_000575 | 17 | NM_000584 | 88 |
| CXCL16 | NP_071342 | 18 | NM_022059 | 89 |
| CXCR1 | NP_000625 | 19 | NM_000634 | 90 |
| CXCR2 | NP_001548 | 20 | NM_001557 | 91 |
| CXCR4a | NP_001008540 | 21 | NM_001008540 | 92 |
| CXCR4b | NP_003458 | 22 | NM_003467 | 93 |
| CXCR6 | NP_006555 | 23 | NM_006564 | 94 |
| CCL1 | NP_002972 | 24 | NM_002981 | 95 |
| CCL2 | NP_002973 | 25 | NM 002982 | 96 |
| CCL3 | NP_002974 | 26 | NM 002983 | 97 |
| CCL4 | NP_002975 | 27 | NM 002984 | 98 |
| CCL4L1 | NP_001001435 | 28 | AY079147 | 99 |
| CCL5 | NP_002976 | 29 | NM 002985 | 100 |
| CCL7 | NP_006264 | 30 | NM 006273 | 101 |
| CCL8 | NP_005614 | 31 | NM 005623 | 102 |
| CCL11 | CAG33702 | 32 | NM_002986 | 103 |
| CCL13 | NP_005399 | 33 | NM_005408 | 104 |
| CCL14-1 | NP_116739 | 34 | NM 032963 | 105 |
| CCL14-2 | NP_116738 | 35 | NM 032962 | 106 |
| CCL15 | NP_116741 | 36 | NM_032965 | 107 |
| CCL16 | NP 004581 | 37 | NM 004590 | 108 |
| CCL17 | NP_002978 | 38 | NM_002987 | 109 |
| CCL18 | NP_002979 | 39 | NM_002988 | 110 |
| CCL19 | NP_006265 | 40 | NM 006274 | 111 |
| CCL20-1 | NP_004582 | 41 | NM 004591 | 112 |
| CCL20-2 | NP_001123518 | 42 | NM_001130046 | 113 |
| CCL22 | NP_002981 | 43 | NM_002990 | 114 |
| CCL23-1 | NP_665905 | 44 | NM_145898 | 115 |
| CCL23-2 | NP_005055 | 45 | NM_005064 | 116 |
| CCL24 | NP_002982 | 46 | NM 002991 | 117 |
| CCL25-1 | NP 005615 | 47 | NM 005624 | 118 |
| CCL25-2 | NP 683686 | 48 | NM_001201359 | 119 |
| CCL25-3 | EAW68951 | 49 | | |
| CCL26 | NP_006063 | 50 | NM 006072 | 120 |
| CCL27 | NP_006655 | 51 | NM_006664 | 121 |
| CCR2-A | NP_001116513 | 52 | NM_001123041 | 122 |
| CCR2-B | NP_001116868 | 53 | NM_001123396 | 123 |
| CCR3-1 | NP_847899 | 54 | NM_001837 | 124 |
| CCR3-2 | NP_847898 | 55 | NM_178328 | 125 |
| CCR3-3 | NP_001158152 | 56 | NM_001164680 | 126 |
| CCR4 | NP_005499 | 57 | NM_005508 | 127 |
| CCR5 | AAB57793 | 58 | NM 000579 | 128 |
| CCR6 | NP_004358 | 59 | U45984 | 129 |
| CCR8 | NP_005192 | 60 | NM_005201 | 130 |
| CCR9A | NP_112477 | 61 | AF145439 | 131 |
| CCR9B | NP_006632 | 62 | AF145440 | 132 |
| CCR10 | NP_057686 | 63 | AF215981 | 133 |
| CCRL1 | NP 057641 | 64 | NM 016557 | 134 |
| CCRL2-1 | NP_003956 | 65 | NM 003965 | 135 |
| CCRL2-2 | NP_001124382 | 66 | NM_001130910 | 136 |
| XCL1 | AAH69817 | 67 | NM_002995 | 137 |
| XCR1 | NP_005274 | 68 | NM_005283 | 138 |
| CX3CR1a | NP_001164645 | 69 | NM_001171174 | 139 |
| CX3CR1b | NP 001328 | 70 | NM 001337 | 140 |
| CX3CL1 | NP 002987 | 71 | NM 002996 | 141 |

In some embodiments, the secondary anti-inflammatory agent binds specifically to a cytokine or cytokine receptor. Exemplary cytokine or cytokine receptor targets and/or their reactive inhibitory products include, but are not limited to, interferon-α, -β, or -γ; tumor necrosis factor (TNF)-alpha, e.g., (infliximab (REMICADE), adalimumab (HUMIRA®), D2E7 (BASF Pharma), and HUMICADE® (Celltech)); soluble forms of the TNF receptor (etanercept (ENBREL®)); CD20, including rituximab (RITUXAN®), humanized 2H7, 2F2 (Hu-Max-CD20), human CD20 antibody (Genmab), and humanized A20 antibody (Immunomedics); TNF-beta; interleukin-2 (IL-2), including daclizumab; IL-2 receptor, interleukin-4 (IL-4) and IL-4 receptor; interleukin-6 (IL-6) and IL-6 receptor; interleukin-1 (IL-1) receptor, including IL-1 receptor agents, such as anakinra (KINERET®); LFA-1, including anti-CD11a, anti-CD18 antibodies, and soluble peptides containing a LFA-3 binding domain; anti-L3T4 antibodies; interleukin-1β (IL-1β); interleukin-8 (IL-8); interferon-γ (IFN-γ); vascular endothelial growth factor (VEGF); leukemia inhibitory factor (LIF); monocyte chemoattractant protein-1 (MCP-1); RANTES; interleukin-10 (IL-10); interleukin-12 (IL-12); matrix metalloproteinase 2 (MMP2); IP-10; macrophage inflammatory protein 1α (MIP1α); macrophage inflammatory protein 1β (MIP1β); pan-T, including anti-CD3 or anti-CD4/CD4a antibodies; BAFF (zTNF4, BLyS) and BAFF receptor, BR3; anti-idiotypic antibodies for MHC antigens and MHC fragments; CD40 receptor and anti-CD40 ligand (CD154); CTLA4-Ig; T-cell receptor antibodies, such as T10B9; heterologous anti-lymphocyte globulin; streptokinase; transforming growth factor-beta (TGF-beta); streptodomase; RNA or DNA from the host; chlorambucil; deoxyspergualin; T-cell receptor; and T-cell receptor fragments.

Anti-Inflammatory Small Molecule Chemical Compounds.

Exemplary small molecule anti-inflammatory agents that can be used as secondary anti-inflammatory agent include, but are not are not limited to, small molecule compounds or medicaments selected from the group consisting of analgesics, such as aspirin or TYLENOL® (Acetaminophen); 2-amino-6-aryl-5-substituted pyrimidines; nonsteroidal anti-inflammatory drugs (NSAIDs), such as acemetacin, amtolmetin, azapropazone, benorilate, benoxaprofen, benzydamine hydrochloride, bromfenal, bufexamac, butibufen, carprofen, celecoxib, choline salicylate, diclofenac dipyone, droxicam, etodolac, etofenamate, etoricoxib, felbinac, fentiazac, floctafenine, ibuprofen, indoprofen, isoxicam, lomoxicam, loxoprofen, licofelone, fepradinol, magnesium salicylate, meclofenamic acid, meloxicam, morniflumate, niflumic acid, nimesulide, oxaprozen, piketoprofen, priazolac, pirprofen, propyphenazone, proquazone, rofecoxib, salalate, sodium salicylate, sodium thiosalicylate, suprofen, tenidap, tiaprofenic acid, trolamine salicylate, zomepirac, aclofenac, aloxiprin, naproxen, aproxen, aspirin, diflunisal, fenoprofen, indomethacin, mefenamic acid, piroxicam, phenylbutazone, salicylamide, salicylic acid, sulindac, desoxysulindac, tenoxicam, tramadol, ketoralac, clonixin, fenbufen, benzydamine hydrochloride, meclofenamic acid, flufenamic acid, or tolmetin; ganciclovir; glucocorticoids such as cortisol or aldosterone; anti-inflammatory agents, such as cyclooxygenase inhibitors; 5-lipoxygenase inhibitors; leukotriene receptor agents; purine agents, such as azathioprine and mycophenolate mofetil (MMF); alkylating agents, such as cyclophosphamide; bromocryptine; danazol; dapsone; glutaraldehyde; cyclosporine; 6 mercaptopurine; corticosteroids, including oral glucocorticosteroids or glucocorticoid analogs, e.g., prednisone; methylprednisolone, including SOLU-MEDROL® and methylprednisolone sodium succinate, triamcinolone, and betamethasone, dexamethasone; aminosalicylate; azathioprine, calcineurin inhibitors, such as cyclosporine, tacrolimus (FK-506), and sirolimus (rapamycin); RS-61443 (mycophenolate mofetil); dihydrofolate reductase inhibitors, such as methotrexate (oral or subcutaneous); anti-malarial agents, such as chloroquine and hydroxychloroquine; sulfasalazine; leflunomide; sulfasalazine (AZULFIDINE); hydroxychloroquine (PLAQUENIL); mitoxantrone (NOVANTRONE®; Immunex Corporation), interferon β-1α (AVONEX®; Ares-Sorono Group), interferonβ-1b (BETASERON®; Berlex Laboratories, Inc.); glatiramer acetate (COPAXONE®; Teva Pharmaceuticals); antibiotics, such as FLAGYL® (metronidazole) or CIPRO® (Ciprofloxacin); and combinations and derivatives thereof.

In some embodiments, the primary anti-inflammatory agent and the secondary anti-inflammatory agent are directed against the same chemokine/chemokine receptor. In other embodiments, the primary anti-inflammatory agent and the secondary anti-inflammatory agent are directed against different chemokine/chemokine receptor. Table 2 describes the association between inflammatory disease and certain chemikine and chemikine receptors.

TABLE 2
Chemokine, Chemokine Receptor and Inflammatory Disease
Association (dependent of stage of disease)

| | | Chemokine |
|---|---|---|
| Disease | Chemokine | Receptor |
| Allergies | CCL1, CCL2, CCL5, CCL7, | CCR3, CCR4, |
| (Skin, Food & | CCL8, CCL11, CCL13, | CCR8, CCR9 |
| Respiratory) | CCL17, CCL22, CCL24, | |
| | CCL25, CCL26 | |
| Asthma | CCL3, CCL4, CCL5, CCL7, | CCR3, CCR4, |
| | CCL8, CCL11, CCL15, | CCR5, |
| | CCL17, CCL22, CCL24, | |
| | CCL26, | |
| Septic Shock, | CXCL1, CXCL2, CXCL3, | CXCR1, CXCR2, |
| Anaphylaxis | CXCL5, CXCL6, CXCL7, | CXCR3 |
| | CXCL8, CXCL9, | |
| | CXCL10, CXCL11, CCL5 | |
| Arthritis | CXCL9, CXCL10, CXCL11, | CXCR3, CXCR4, |
| (septic, rheumatoid, | CXCL12, CXCL13 | CXCR5 |
| psoriatic) | CCL20 | CCR6 |
| | XCL1 | XCR1 |
| | CX3CL1 | CX3CR1 |
| Osteoarthritis | CXCL1, CXCL2, CXCL3, | CXCR1, CXCR2, |
| | CXCL5, CXCL6, CXCL7, | CCR2, CCR5 |
| | CXCL8, CXCL12, CXCL13, | |
| | CCL2, CCL3, CCL4, CCL7, | |
| | CCL8, CCL13, CCL5, CCL18 | |
| Atherosclerosis | CXCL1, CXCL2, CXCL3, | CXCR1, CXCR2 |
| | CXCL4, CXCL5, CXCL8 | |
| | CCL2, CCL3, CCL4, CCL8, | CCR2, CCR8 |
| | CCL12, CCL13, CCL17, | |
| | CCL22 CX3CL1 | CX3CR1 |
| Dermatitis & | CXCL9, CXCL10, CXCL11, | CXCR3 |
| Delayed-Typed | CCL2, CCL3, CCL4, CCL5, | CCR4, CCR5, |
| Hypersensitivity | CCL17, CCL20, CCL22, | CCR6, CCR10 |
| Diabetes | CCL27 CXCL9, CXCL10, | CXCR3 |
| | CXCL11, CCL2, CCL9 | CCR2, CCR4 |
| | CX3CL1 | CX3CR1 |
| Graft rejection | CXCL9, CXCL10, CXCL11, | CXCR3 |
| | CCL3, CCL4, CCL5 | CCR5 |
| | XCL1 | XCR1 |
| Inflammatory | CXCL9, CXCL10, CXCL11, | CXCR3 |
| Bowel Diseases | CCL3, CCL4, CCL5 | CCR5 |
| Interstitial Cystitis | CXCL9, CXCL10, CXCL11, | CXCR3 |
| | CCL3, CCL4, CCL5 | CCR5 |
| Multiple Sclerosis | CXCL9, CXCL10, CXCL11, | CXCR3 |
| | CCL3, CCL4, CCL5, CCL7, | CCR1, CCR5 |
| | CCL14, CCL15, CCL23 | |
| Myasthemia gravis, | CXCL9, CXCL10, CXCL11, | CXCR3 |
| Grave's disease, | CCL3, CCL4, CCL5 | CCR5 |
| & Hashimoto | XCL1 | XCR1 |
| thyroiditis | | |
| Nephritis & | CXCL9, CXCL10, CXCL11, | CXCR3, CXCR5 |
| Systemic Lupus | CXCL13 | |
| Erthematosus | CCL2, CCL3, CCL4, CCL5, | CCR2, CCR4 |
| | CCL8, CCL12, CCL13, | CX3CR1 |
| | CX3CL1 | |
| Pneumonitis, | CXCL1, CXCL2, CXCL3, | CXCR2, CXCR3 |
| Chronic | CXCL5, CXCL7, CXCL8 | |
| Obstructive | CCL3, CCL5, CCL7, CCL8, | CCR3 |
| Pulmonary Disease, | CCL11, CCL13, CCL24, | |
| & Chronic | CCL26. | |
| Bronchitis | | |

### Administration of Anti-Inflammatory Agents

The anti-inflammatory agents may be administered to the subject with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In certain embodiments, the anti-inflammatory agent(s) may be administered directly to an inflammed tissue. For example, in the case of inflammatory bowel disorders, mucosal tissue may be directly contacted with the anti-inflammatory agent(s). For skin inflammatory diseases such as psoriasis, dermal tissue may be contacted directly with the anti-inflammatory agent(s) in a cream, lotion, or ointment. For asthma, pulmonary tissue, e.g., bronchoalveolar tissue may be contacted by inhalation of a liquid or powder aspirate. The anti-inflammatory agent may also be placed on a solid support such as a sponge or gauze for administration against the target chemokine to the affected tissues.

The anti-inflammatory agents of the instant application can be administered in the usually accepted pharmaceutically acceptable carriers. Acceptable carriers include, but are not limited to, saline, buffered saline, and glucose in saline. Solid supports, liposomes, nanoparticles, microparticles, nanospheres or microspheres may also be used as carriers for administration of the anti-inflammatory agents.

The appropriate dosage (“therapeutically effective amount”) of the anti-inflammatory agents will depend, for example, on the condition to be treated, the severity and course of the condition, the mode of administration, whether the antibody or agent is administered for preventive or therapeutic purposes, the bioavailability of the particular agent(s), previous therapy, the age and weight of the patient, the patient's clinical history and response to the antibody, the type of the anti-inflammatory agent used, discretion of the attending physician, etc. The anti-inflammatory agent is suitably administered to the patent at one time or over a series of treatments and may be administered to the patient at any time from diagnosis onwards. The anti-inflammatory agent may be administered as the sole treatment or in conjunction with other drugs or therapies useful in treating the condition in question.

As a general proposition, the therapeutically effective amount of the anti-inflammatory agent (e.g., antibodies and/or anti-inflammatory small molecule compounds) is administered will be in the range of about 1 ng/kg body weight/day to about 100 mg/kg body weight/day whether by one or more administrations. In a particular embodiments, each anti-inflammatory agent is administered in the range of from about 1 ng/kg body weight/day to about 10 mg/kg body weight/day, about 1 ng/kg body weight/day to about 1 mg/kg body weight/day, about 1 ng/kg body weight/day to about 100 μg/kg body weight/day, about 1 ng/kg body weight/day to about 10 μg/kg body weight/day, about 1 ng/kg body weight/day to about 1 μg/kg body weight/day, about 1 ng/kg body weight/day to about 100 ng/kg body weight/day, about 1 ng/kg body weight/day to about 10 ng/kg body weight/day, about 10 ng/kg body weight/day to about 100 mg/kg body weight/day, about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 μg/kg body weight/day to about 100 mg/kg body weight/day, about 1 μg/kg body weight/day to about 10 mg/kg body weight/day, about 1 μg/kg body weight/day to about 1 mg/kg body weight/day, about 1 μg/kg body weight/day to about 100 μg/kg body weight/day, about 1 μg/kg body weight/day to about 10 μg/kg body weight/day, about 10 μg/kg body weight/day to about 100 mg/kg body weight/day, about 10 μg/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In other embodiments, the anti-inflammatory agent (e.g., antibodies and/or anti-inflammatory small molecule compounds) is administered at a dose of 500 μg to 20 g every three days, or 25 mg/kg body weight every three days.

In other embodiments, each anti-inflammatory agent is administered in the range of about 10 ng to about 100 ng per individual administration, about 10 ng to about 1 μg per individual administration, about 10 ng to about 10 μg per individual administration, about 10 ng to about 100 μg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 μg per individual administration, about 100 ng to about 10 μg per individual administration, about 100 ng to about 100 μg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 μg to about 10 μg per individual administration, about 1 μg to about 100 μg per individual administration, about 1 μg to about 1 mg per individual administration, about 1 μg to about 10 mg per individual administration, about 1 μg to about 100 mg per individual administration, about 1 μg to about 1000 mg per injection, about 1 μg to about 10,000 mg per individual administration, about 10 μg to about 100 μg per individual administration, about 10 μg to about 1 mg per individual administration, about 10 μg to about 10 mg per individual administration, about 10 μg to about 100 mg per individual administration, about 10 μg to about 1000 mg per injection, about 10 μg to about 10,000 mg per individual administration, about 100 μg to about 1 mg per individual administration, about 100 μg to about 10 mg per individual administration, about 100 μg to about 100 mg per individual administration, about 100 μg to about 1000 mg per injection, about 100 μg to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The chemotherapeutic agent contained in the PBM nanoparticles may be administered daily, or every 2, 3, 4, 5, 6 and 7 days, or every 1, 2, 3 or 4 weeks.

In other particular embodiments, the amount of the anti-inflammatory agent administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day. As expected, the dosage will be dependant on the condition, size, age and condition of the patient.

Dosage can be tested in several animal models that can partially mimic chronic ulcerative colitis. The most widely used model is the 2,4,6-trinitrobenesulfonic acid/ethanol (TNBS) induced colitis model, which induces chronic inflammation and ulceration in the colon. When TNBS is introduced into the colon of susceptible mice via intra-rectal instillation, it induces T-cell mediated immune response in the colonic mucosa, in this case leading to a massive mucosal inflammation characterized by the dense infiltration of T-cells and macrophages throughout the entire wall of the large bowel. Moreover, this histopathologic picture is accompanied by the clinical picture of progressive weight loss (wasting), bloody diarrhea, rectal prolapse, and large bowel wall thickening.

Another colitis model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. These changes are thought to develop due to a toxic effect of DSS on the epithelium and by phagocytosis of lamina propria cells and production of TNF-alpha and IFN-gamma. Despite its common use, several issues regarding the mechanisms of DSS about the relevance to the human disease remain unresolved. DSS is regarded as a T cell-independent model because it is observed in T cell-deficient animals such as SCID mice.

The administration of the anti-inflammatory agent of the present application can be evaluated in the TNBS or DSS models for amelioration of gastrointestinal disease. CXCL9, CXCL10, CXCL11, CXCL13, CXCR3 and CXCR5 are believed to play a role in the inflammatory response in inflammatory bowel disorders, including colitis, and the neutralization of CXCL9, CXCL10, CXCL11, CXCL13, CXCR3 and CXCR5 activity by administrating the anti-inflammatory agent of the present application can provide a potential therapeutic approach for gastrointestinal inflammatory diseases, including IBD.

As shown in the Table 2, the particular chemokines which give rise to inflammatory diseases differ with the disease. They also differ among individuals. Hence, it is wise, when treating an individual, to identify the particular chemokines which are increased in the tissues of the patient. By exposing patient tissue samples to the particular antibodies against each of the chemokines and evaluating the amount of antibody/chemokine binding, it is possible to evaluate the level of expression for each chemokine to enable a determination of the appropriate type and amount of antibodies to administer for a given inflammatory disease.

The antibody may be administered, as appropriate or indicated, a single dose as a bolus or by continuous infusion, or as multiple doses by bolus or by continuous infusion. Multiple doses may be administered, for example, multiple times per day, once daily, every 2, 3, 4, 5, 6 or 7 days, weekly, every 2, 3, 4, 5 or 6 weeks or monthly. However, other dosage regimens may be useful. The progress of this therapy is easily monitored by conventional techniques.

### Compositions and Kits for Treating or Preventing Inflammatory Conditions

Another aspect of the present application relates to compositions and kits for treating or preventing inflammatory conditions. In one embodiment, the composition comprises an anti-inflammatory agent capable of (1) inhibiting the expression of CXCL9, CXCL10, CXCL11, CXCL13, CXCR5 and/or CXCR3; (2) inhibiting the interaction between any one of CXCL9, CXCL10, CXCL11, CXCL13, CXCR5 and/or CXCR3, or (3) inhibiting a biological activity of CXCL9, CXCL10, CXCL11, CXCL13, CXCR5 and/or CXCR3, wherein the anti-inflammatory agent is an antibody, antibody fragment, short interfering RNA (siRNA), aptamer, synbody, binding agent, peptide, aptamer-siRNA chimera, single stranded antisense oligonucleotide, triplex forming oligonucleotide, ribozyme, external guide sequence, agent-encoding expression vector, and a pharmaceutically acceptable carrier.

The composition of the present invention may contain a single type of antibody directed against any one of CXCL9, CXCL10, CXCL11, CXCL13, CXCR5 and CXCR3, or two or more antibodies directed against the same chemokine or chemokine receptor, different chemokines or chemokine receptors, or combinations thereof as described above. The composition may also contain therapeutically effective amounts of other anti-inflammatory agents as described above.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions. In certain embodiments, the pharmaceutically acceptable carrier comprises serum albumin.

The pharmaceutical composition of the invention is formulated to be compatible with its intended route of administration. Examples of routes of administration include parenteral, e.g., intrathecal, intra-arterial, intravenous, intradermal, subcutaneous, oral, transdermal (topical) and transmucosal administration.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a neuregulin) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. The pharmaceutical compositions can be included in a container, pack, or dispenser together with instructions for administration.

The present invention is further illustrated by the following examples that should not be construed as limiting. The contents of all references, patents, and published patent applications cited throughout this application, as well as the Figures and Tables, are incorporated herein by reference.

### Example 1

### Upregulation of Chemokines and their Receptors in Inflammatory Diseases

### Materials and Methods

Primer Design. Messenger RNA sequences for CXCL9, CXCL10, CXCL11, CCRL1, CCRL2, CCR5, CCL1, CCL2, CCL3, CCL4, CCL4L1, CCL5, CCL7, CCL8, CCL14-1, CCL14-2, CCL14-3, CCL15-1, CCL15-2, CCL16, CCL19, CCL23-1, CCL23-2, CCL24, CCL26, CCR6, CCL20, and CCL25, CCL25-1, CCL25-2 were obtained from the NIH-NCBI gene bank database (Table 1). Primers were designed using the BeaconJ 2.0 computer program. Thermodynamic analysis of the primers was conducted using computer programs: Primer Premier) and MIT Primer 3. The resulting primer sets were compared against the entire human genome to confirm specificity.

Real Time PCR Analysis.

Lymphocytes or inflamed tissues were cultured in RMPI-1640 containing 10% fetal calf serum, 2% human serum, supplemented with non-essential amino acids, L-glutamate, and sodium pyruvate (complete media). Additionally, primary inflammatory and normal-paired matched tissues were obtained from clinical isolates (Clinomics Biosciences, Frederick, Md. and UAB Tissue Procurement, Birmingham, Ala.). Messenger RNA (mRNA) was isolated from 10⁶ cells using TriReagent (Molecular Research Center, Cincinnati, Ohio) according to manufacturers protocols. Potential genomic DNA contamination was removed from these samples by treatment with 10 U/μl of RNase free DNase (Invitrogen, San Diego, Calif.) for 15 minutes at 37° C. RNA was then precipitated and resuspended in RNA Secure (Ambion, Austin, Tex.). cDNA was generated by reverse transcribing approximately 2 μg of total RNA using Taqman7 reverse transcription reagents (AppliedBiosystems, Foster City, Calif.) according to manufacturers protocols. Subsequently, cDNAs were amplified with specific human cDNA primers, to CXCL9, CXCL10, CXCL11, CCRL1, CCRL2, CCR5, CCL1, CCL2, CCL3, CCL4, CCL4L1, CCL5, CCL7, CCL8, CCL14-1, CCL14-2, CCL14-3, CCL15-1, CCL15-2, CCL16, CCL19, CCL23-1, CCL23-2, CCL24, CCL26, CCR6, CCL20, and CCL25, CCL25-1, CCL25-2, using SYBR7 Green PCR master mix reagents (Applied Biosystems) according to manufacturers protocol. The level of copies of mRNA of these targets were evaluated by real-time PCR analysis using the BioRad Icycler and software (Hercules, Calif.).

Anti-Sera Preparation.

The 15 amino acid peptides from chemokines CXCL9, CXCL10, CXCL11, CCRL1, CCRL2, CCR5, CCL1, CCL2, CCL3, CCL4, CCL4L1, CCL5, CCL7, CCL8, CCL14-1, CCL14-2, CCL14-3, CCL15-1, CCL15-2, CCL16, CCL19, CCL23-1, CCL23-2, CCL24, CCL26, CCR6, CCL20, and CCL25, CCL25-1, CCL25-2 (Table 1) were synthesized (Sigma Genosys, The Woodlands, Tex.) and conjugated to hen egg lysozyme (Pierce, Rockford, Ill.) to generate the antigens for subsequent immunizations for anti-sera preparation or monoclonal antibody generation. The endotoxin levels of chemokine peptide conjugates were quantified by the chromogenic Limulus amebocyte lysate assay (Cape Cod, Inc., Falmouth, Miss.) and shown to be <5 EU/mg. 100 μg of the antigen was used as the immunogen together with complete Freund's adjuvant Ribi Adjuvant system (RAS) for the first immunization in a final volume of 1.0 ml. This mixture was administered in 100 ml aliquots on two sites of the back of the rabbit subcutaneously and 400 ml intramuscularly in each hind leg muscle. Three to four weeks later, rabbits received 100 μg of the antigen in addition to incomplete Freund's adjuvant for 3 subsequent immunizations. Anti-sera were collected when antibody titers reached 1:1,000,000. Subsequently, normal or anti-sera were heat-inactivated and diluted 1:50 in PBS.

Monoclonal Antibody Preparation.

The 15 amino acid peptides from chemokines CXCL9, CXCL10, CXCL11, CCRL1, CCRL2, CCR5, CCL1, CCL2, CCL3, CCL4, CCL4L1, CCL5, CCL7, CCL8, CCL14-1, CCL14-2, CCL14-3, CCL15-1, CCL15-2, CCL16, CCL19, CCL23-1, CCL23-2, CCL24, CCL26, CCR6, CCL20, and CCL25, CCL25-1, CCL25-2 (Sequences 1 through 30) were synthesized (Sigma Genosys) and conjugated to hen egg lysozyme (Pierce) to generate the Antigen@ for subsequent immunizations for anti-sera preparation or monoclonal antibody generation. The endotoxin levels of chemokine peptide conjugates were quantified by the chromogenic Limulus amebocyte lysate assay (Cape Cod, Inc., Falmouth, Miss.) and shown to be <5 EU/mg. 100 μg of the antigen was used as the immunogen together with complete Freund's adjuvant Ribi Adjuvant system (RAS) for the first immunization in a final volume of 200 μl. This mixture was subcutaneously administered in 100 μl aliquots at two sites of the back of a rat, mouse, or immunoglobulin-humanized mouse. Two weeks later, animals received 100 μg of the antigen in addition to incomplete Freund's adjuvant for 3 subsequent immunizations. Serum were collected and when anti-CXCL9, -CXCL10, -CXCL11, -CCRL1, -CCRL2, -CCR5, -CCL1, -CCL2, -CCL3, -CCL4, -CCL4L1, -CCL5, -CCL7, -CCL8, -CCL14-1, -CCL14-2, -CCL14-3, -CCL15-1, -CCL15-2, -CCL16, -CCL19, -CCL23-1, -CCL23-2, -CCL24, -CCL26, -CCR6, -CCL20, and -CCL25, -CCL25-1, -CCL25-2 antibody titers reached 1:2,000,000, hosts were sacrificed and splenocytes were isolated for hybridoma generation.

B cells from the spleen or lymph nodes of immunized hosts were fused with immortal myeloma cell lines (e.g., YB2/0). Hybridomas were next isolated after selective culturing conditions (i.e., HAT-supplemented media) and limiting dilution methods of hybridoma cloning. Cells that produce antibodies with the desired specificity were selected using ELISA. Hybridomas from normal rats or mice were humanized with molecular biological techniques in common use. After cloning a high affinity and prolific hybridoma, antibodies were isolated from ascites or culture supernatants and adjusted to a titer of 1:2,000,000 and diluted 1:50 in PBS.

Anti-Sera or Monoclonal Antibody Treatment.

Knockout or transgenic mice (8 to 12 weeks old, Charles River Laboratory, Wilmington, Mass.) that spontaneous—or when treated—develop inflammatory diseases were treated with 200 μl intraperitoneal injections of either anti-sera or monoclonal antibodies specific for each of the chemokines every three days. The inflammatory disease state of the host was next monitored for progression or regression of disease.

Cytokine Analysis by ELISA.

The serum level of IL-2, -IL-6, -TNF-α, and -IFN-γ were determined by ELISA, following the manufacturers instructions (E-Biosciences, San Diego, Calif.). Plates were coated with 100 μl of the respective capture antibody in 0.1 M bicarbonate buffer (pH 9.5) and incubated O/N at 4° C. After aspiration and washing with wash buffer, the wells were blocked with assay diluent for 1 hour at RT. Samples and standards were added and incubated for 2 hours at RT. Next, 100 μl of detection antibody solutions were added and incubated for 1 hour. 100 μl of avidin-HRP solution was added and incubated for 30 minutes. Subsequently, 100 μl Tetramethylbenzidine (TMB) substrate solution was added and allowed to react for 20 minutes. 50 μl of the stop solution was added and plates were read at 450 nm. The cytokine ELISA assays were capable of detecting >15 pg/ml for each assay.

Cytokine Analysis by Multiplex Cytokine ELISA.

The T helper cell derived cytokines, IL-1α, IL-1β, IL-2, IL-12, IFN-γ, TNF-α, in serum were also determined by Beadlyte mouse multi-cytokine detection system kit provided by BioRad, following manufacturer instructions. Filter bottom plates were rinsed with 100 μl of bio-plex assay buffer and removal using a Millipore Multiscreen Separation Vacuum Manifold System (Bedford, Mass.), set at 5 in Hg. IL-1α, IL-1β, IL-2; IL-12, IFN-γ, TNF-α beads in assay buffer were added into wells. Next, 50 μl of serum or standard solution were added and the plates were incubated for 30 minutes at RT with continuous shaking (setting 3) using a Lab-Line Instrument Titer Plate Shaker (Melrose, Ill.), after sealing the plates. The filter bottom plates were washed 2 times, as before, and centrifuged at 300×g for 30 seconds. Subsequently, 50 μl of anti-mouse IL-1α, IL-1β, IL-2, IL-12, IFN-γ, TNF-α antibody-biotin reporter solution was added in each well followed by incubation with continuous shaking for 30 minutes followed by centrifugation at 300×g for 30 seconds. The plates were washed 3 times with 100 μl of bio-plex assay buffer as before. Next, 50 μl streptavidin-phycoerythrin solution was added to each well and incubated with continuous shaking for 10 minute at RT. 125 μl of bio-plex assay buffer was added and Beadlyte readings were measured using a Luminexl instrument (Austin, Tex.). The resulting data was collected and calculated using Bio-plexl software (Bio-Rad). The cytokine Beadlyte assays were capable of detecting >5 pg/ml for each analyte.

Serum Amyloid Protein A (BAA) ELISA.

The SAA levels were determined by ELISA using a kit supplied by Biosource International, (Camarillo, Calif.). Briefly, 50 μl of SAA-specific monoclonal antibody solution was used to coat micro-titer strips to capture SAA. Serum samples and standards were added to wells and incubated for 2 hours at RT. After washing in the assay buffer, the HRP-conjugated anti-SAA monoclonal antibody solution was added and incubated for 1 hour at 37° C. After washing, 100 μl Tetramethylbenzidine (TMB) substrate solution was added and the reaction was stopped after incubation for 15 minutes at RT. After the stop solution was added, the plates were read at 450 nm.

Histology and Pathology Scoring.

Fixed tissues were sectioned at 6 μm, and stained with hematoxylin and eosin for light microscopic examination. The intestinal lesions were multi-focal and of variable severity, the grades given to any section of intestine took into account the number of lesions as well as their severity. A score (0 to 4) was given, based on the following criteria: (Grade 0) no change from normal tissue. (Grade 1) 1 or a few multi-focal mononuclear cell infiltrates, minimal hyperplasia and no depletion of mucus. (Grade 2) lesions tended to involve more of the mucosa and lesions had several multi-focal, yet mild, inflammatory cell infiltrates in the lamina propria composed of mononuclear cells, mild hyperplasia, epithelial erosions were occasionally present, and no inflammation was noticed in the sub-mucosa. (Grade 3) lesions involved a large area of mucosa or were more frequent than Grade 2, where inflammation was moderate and often involved in the sub-mucosa as well as moderate epithelial hyperplasia, with a mixture of mononuclear cells and neutrophils. (Grade 4) lesions usually involved most of the section and were more severe than Grade 3 lesions. Additionally, Grade 4 inflammations were more severe and included mononuclear cell and neutrophils; epithelial hyperplasia was marked with crowding of epithelial cells in elongated glands. The summation of these score provide a total inflammatory disease score per mouse. The disease score could range from 0 (no change in any segment) to a maximum of 12 with Grade 4 lesions of segments.

Data Analysis.

SigmaStat 2000 (Chicago, Ill.) software was used to analyze and confirm the statistical significance of data. The data were subsequently analyzed by the Student's t-test, using a two-factor, unpaired test. In this analysis, treated samples were compared to untreated controls. The significance level was set at p<0.05.

### Results

Semiquantitative RT-PCR Identification of Molecular Targets.

RT-PCR products obtained using CXCL9-, CXCL10-, CXCL11-, CCRL1-, CCRL2-, CCR5-, CCL1-, CCL2-, CCL3-, CCL4-, CCL4L1-, CCL5-, CCL7-, CCL8-, CCL14-1-, CCL14-2-, CCL14-3-, CCL15-1-, CCL15-2-, CCL16-, CCL19-, CCL23-1-, CCL23-2-, CCL24-, CCL26-, CCR6-, CCL20-, and CCL25-, CCL25-1-, CCL25-2-specific primer sets did not cross react with other gene targets due to exclusion of primers that annealed to host sequences. The primers used produced different size amplicon products relative the polymorphisms that resulted in CCL4 versus CCL4L1, CCL14-1, CCL14-2, versus CCL14-3, CCL15-1 versus CCL15-2, CCL23-1 versus CCL23-2, and CCL25, CCL25-1, versus CCL25-2. To this end, RT-PCR analysis of tissue from subjects exhibiting anaphylaxis, arthritis (e.g., rheumatoid, psoriatic), asthma, allergies (e.g., drug, insect, plant, food), atherosclerosis, delayed type hypersensitivity, dermatitis, diabetes (e.g., mellitus, juvenile onset), graft rejection, inflammatory bowel diseases (e.g., Crohn's disease, ulcerative colitis, enteritis), multiple sclerosis, myasthemia gravis, pneumonitis, psoriasis, nephritis, rhinitis, spondyloarthropathies, scheroderma, systemic lupus, or thyroiditis revealed that CXCL9, CXCL10, CXCL11, CCRL1, CCRL2, CCR5, CCL1, CCL2, CCL3, CCL4, CCL4L1, CCL5, CCL7, CCL8, CCL14-1, CCL14-2, CCL14-3, CCL15-1, CCL15-2, CCL16, CCL19, CCL23-1, CCL23-2, CCL24, CCL26, CCR6, CCL20, and CCL25, CCL25-1, CCL25-2 were differentially expressed by inflammatory host cells.

In Vivo Inflammatory Disease Inhibition.

Mammals that develop anaphylaxis, septic shock, arthritis (e.g., rheumatoid, psoriatic), asthma, allergies (e.g., drug, insect, plant, food), atherosclerosis, bronchitis, chronic pulmonary obstructive disease, delayed type hypersensitivity, dermatitis, diabetes (e.g., mellitus, juvenile onset), graft rejection, Grave's disease, Hashimoto's thyroiditis, inflammatory bowel diseases (e.g., Crohn's disease, ulcerative colitis, enteritis), interstitial cystitis, multiple sclerosis, myasthemia gravis, pneumonitis, psoriasis, nephritis, rhinitis, spondyloarthropathies, scheroderma, systemic lupus erythematosus, or thyroiditis were allowed to develop the inflammatory disease of interest. Antibodies directed against CXCL9, CXCL10, CXCL11, CCRL1, CCRL2, CCR5, CCL1, CCL2, CCL3, CCL4, CCL4L1, CCL5, CCL7, CCL8, CCL14-1, CCL14-2, CCL14-3, CCL15-1, CCL15-2, CCL16, CCL19, CCL23-1, CCL23-2, CCL24, CCL26, CCR6, CCL20, or CCL25, CCL25-1, CCL25-2 differentially affected the progression and regression of inflammatory disease as determined by histological scoring and comparing pre- and post-treatment serum levels of IFN-γ, IL-1α, IL-1β, IL-6, IL-12, TNF-α, amyloid protein A. Antibodies directed towards CXCL9, CXCL10, CXCL11, CCRL1, CCRL2, CCR5, CCL1, CCL2, CCL3, CCL4, CCL4L1, CCL5, CCL7, CCL8, CCL14-1, CCL14-2, CCL14-3, CCL15-1, CCL15-2, CCL16, CCL19, CCL23-1, CCL23-2, CCL24, CCL26, CCR6, CCL20, or CCL25, CCL25-1, CCL25-2 effectively lead to the both regression and impeding progression of inflammatory disease as determined by histological scoring and comparing pre- and post-treatment serum levels of IFN-γ, IL-1α, IL-1β, IL-6, IL-12, TNF-α, amyloid protein A.

As indicated previously, the chemokines used in the methods of the invention are known. Their accession numbers for the protein sequences are identified in Table 1.

As shown in the table, the particular chemokines which give rise to inflammatory diseases differ with the disease. They also differ among individuals. Hence, it is wise, when treating an individual, to identify the particular chemokines which are increased in the tissues of the patient. Using the antibodies produced against each of the chemokines and exposing the tissue samples from the patient to the particular antibodies, then evaluating the amount of antibody/chemokine binding, it is possible to evaluate the level of expression for each chemokine and to administer to the patient the particular antibodies that will bind the excessive chemokine. This tailored approach to treatment of inflammatory disease is novel, and a particularly valuable aspect of the invention.

### Example 2

### mRNA Expression of IFN-γ, CXCL10, MIG, I-TAC, CXCR3 in Murine Colitis

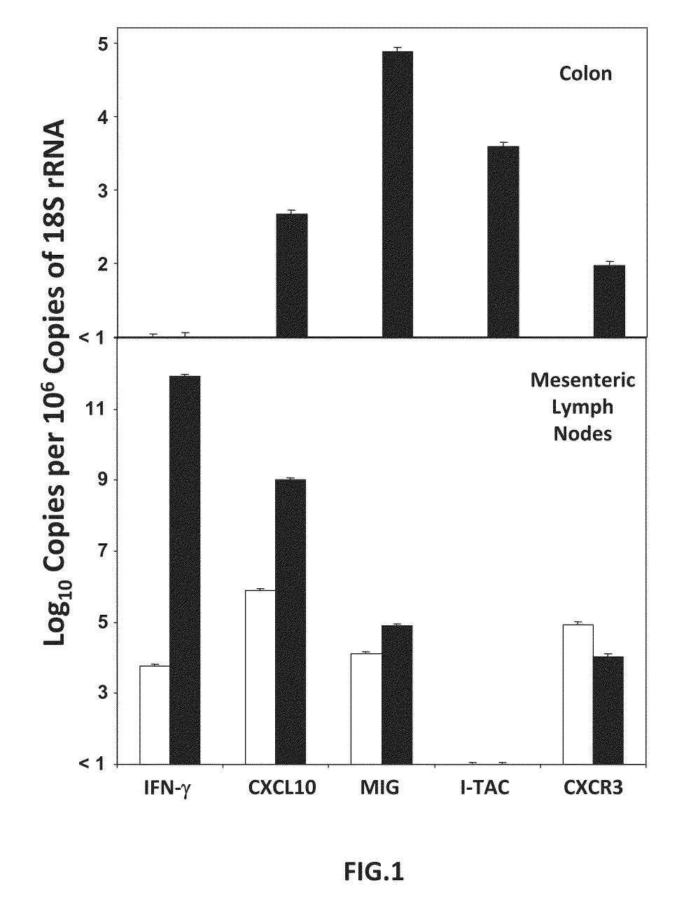

FIG. 1 shows mRNA expression of IFN-γ, CXCL10, MIG, I-TAC, and CXCR3 during murine colitis. Laminar flow barriers were removed from the housing cages of IL-10^{-/-} mice, on C57BL/6 background, for the spontaneous development of colitis. Following sacrifice, total RNA was isolated from the colon or mesenteric lymph nodes from mice before the onset of colitis (sterile conditions, open squares) and after the development of colitis (closed squares). The levels of IFN-γ, IP-10, MIG, I-TAC, and CXCR3 mRNA expression were ascertained after RT-PCR analysis that was capable of detecting >20 copies of transcribed cDNA. In FIG. 1, the Log₁₀ copies of transcripts are expressed relative to actual copies of 18S rRNA.

As shown in FIG. 1, a significant increase in CXCR3 and CXCL10 expression was observed in inflamed colons of IL-10^{-/-} mice developing colitis. In addition, a significant increase in CXCL10 expression was observed in mesenteric lymph nodes of the IL-10^{-/-} mice developing colitis.

### Example 3

### Histological Analysis of IBD in TCR β×δ^{-/-} Mice That Received CD45RB^{HI} or CXCR3⁺ CD4⁺ T Cells by Adoptive Transfer

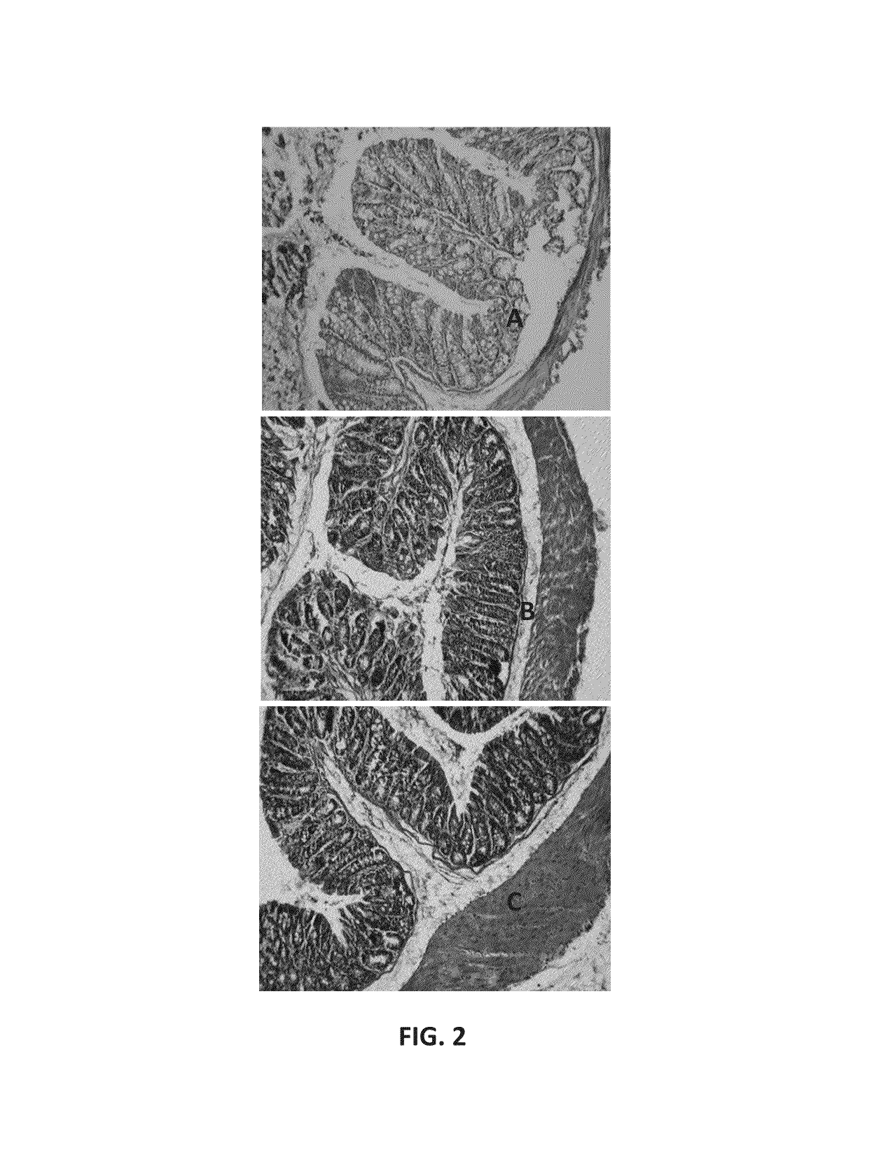

FIG. 2 shows histological analysis of IBD in TCR β×δ^{-/-} mice that received CD45RB^{HI} or CXCR3⁺ CD4⁺ T cells by adoptive transfer. 60× magnification of intestinal inflammation in TCR β×δ^{-/-} mice that received CD45RB^{Lo}—(Panel A), CD45RB^{Hi}—(Panel B), or CXCR3⁺ CD4⁺ T cells (Panel C) from normal C57BL/6 mice. Cross sections of intestines demonstrate the differences in wall thickness, enlargement of mucosal layer, crypt malformation, and leukocyte infiltration using hematoxylin-eosin staining of 6 μm paraffin sections.

This analysis shows that CXCR3⁺ CD4⁺ T cells, which consisted of both CD45RB populations induced induction of colitis in TCR β×δ^{-/-} mice (Panel C).

### Example 4

### SAA Levels and the Development of Colitis in IL-10^{-/-} Mice

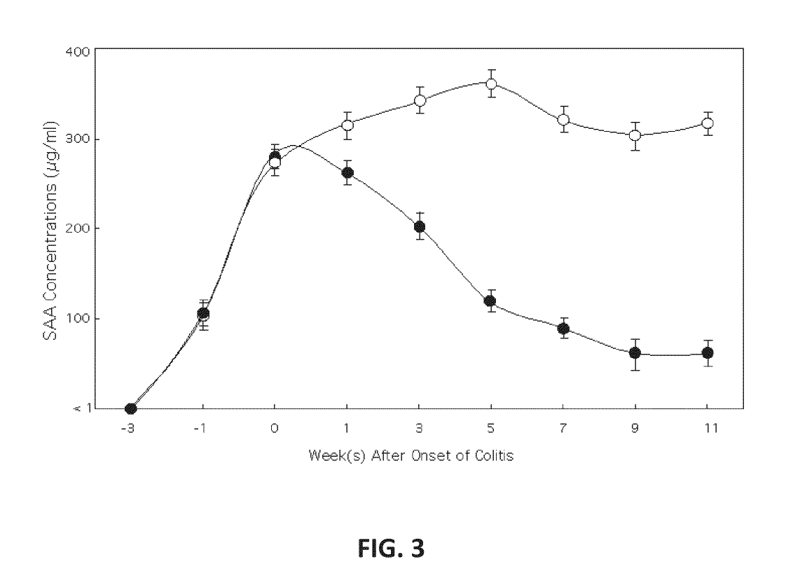

FIG. 3 shows serum amyloid A (SAA) levels and the development of colitis in IL-10^{-/-} mice. SAA concentrations >200 μg/ml were associated with the onset of asymptomatic colitis at week 0. Mice received 200 μl of pre-immune—(open circles) or anti-mouse CXCL10 (closed circles) Ab solutions every 3 days. Sera were collected every 2 weeks and the data presented are the mean SAA concentrations±SEM.

The results in FIG. 3 show that CXCL10 blockage with anti-mouse CXCL10 antibodies inhibited the elevated SAA levels that are associated with IBD.

### Example 5

### Changes in Body Weight of IL-10^{-/-} Mice

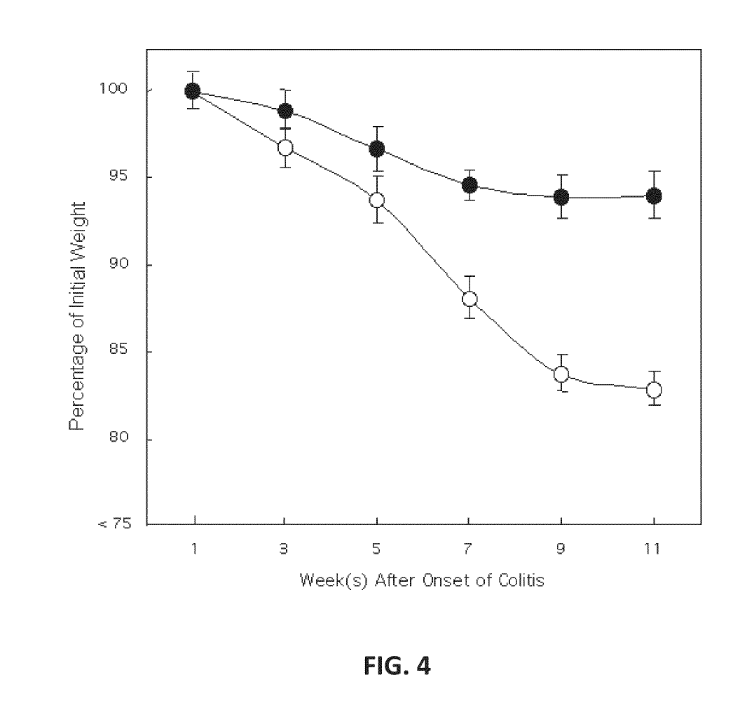

FIG. 4 shows changes in body weight of IL-10^{-/-} mice. The wasting disease associated with murine CD was observed by monitoring the change in initial body mass at week 0. IL-10^{-/-} mice received 200 μl of pre-immune—(open circles) or anti-mouse CXCL10 (closed circles) Ab solutions every 3 days. Body masses were recorded every 2 weeks and the change from initial body mass was expressed as a percentage: weight at week 0 minus weight at week 1, 3, 5, 7, 9, or 11 divided by the weight at week 0.

The results in FIG. 4 show that CXCL10 blockage with anti-mouse CXCL10 antibodies inhibited the weight loss associated with IBD.

### Example 6

### Association of Serum IL-6 and SAA Levels with Murine Colitis

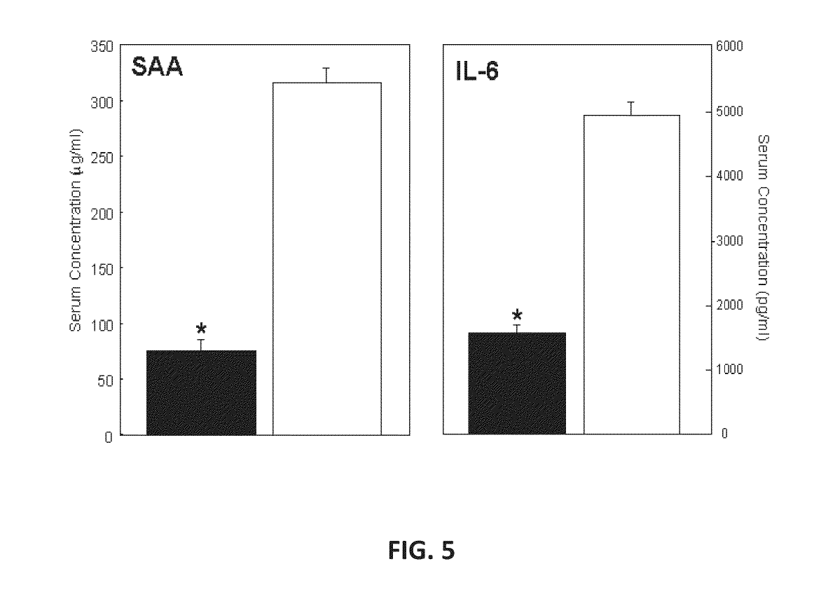

FIG. 5 shows association of serum IL-6 and SAA levels with murine colitis. IL-10^{-/-} mice received 200 μl of pre-immune—(open boxes) or anti-mouse CXCL10 (closed boxes) Ab solutions every 3 days. The levels of SAA and serum IL-6, at week 11, were determined by ELISA. The data presented are the mean SAA or IL-6 concentrations±SEM.

The results in FIG. 5 show that CXCL10 blockage with anti-mouse CXCL10 antibodies significantly reduced SAA and IL-6 serum concentrations as compared with control mice. The results further suggest a utility of using SAA levels as an indicator for the switch from acute (i.e. asymptomatic) to chronic colitis in this murine model of CD.

### Example 7

### Total Fecal and Serum Ab Levels in IL-10^{-/-} Mice

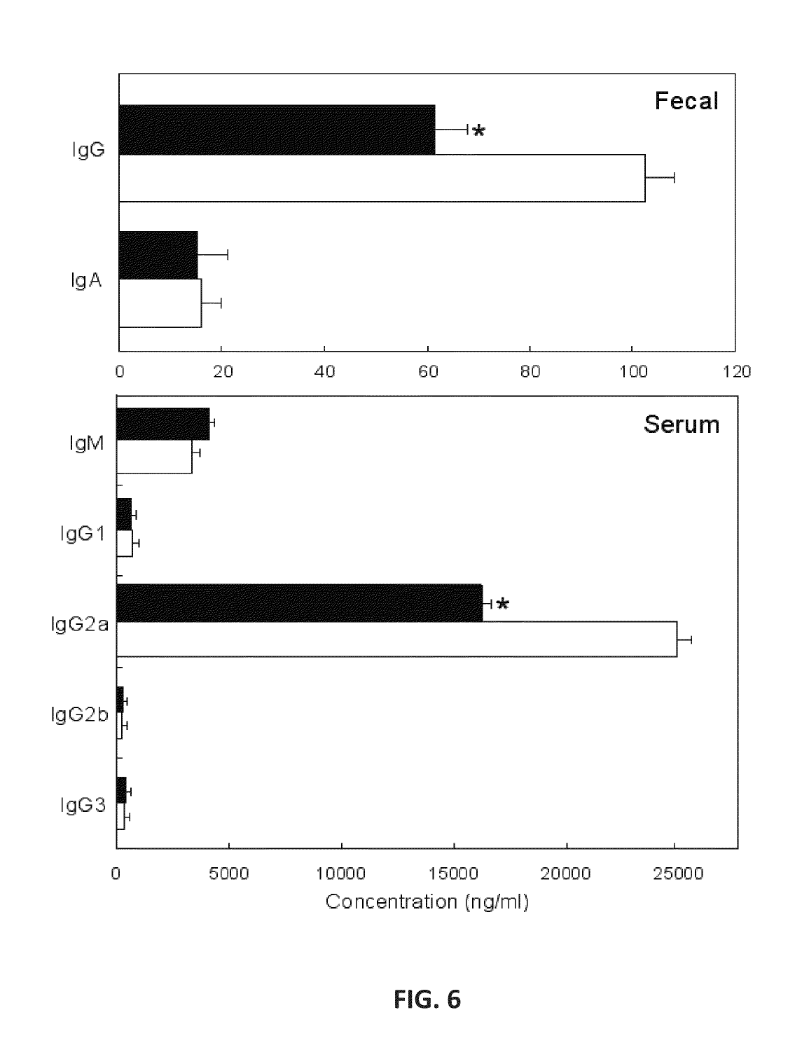

FIG. 6 shows total fecal and serum Ab levels in IL-10^{-/-} mice. Groups of 5 IL-10−/− mice received 200 μl of either pre-immune—(open squares) or anti-mouse IP-10—(closed squares) Ab solutions every 3 days. The data presented are the mean concentration of total Ig Abs (ng/ml)±SEM. Total IgA and IgG Abs in fecal extracts or IgM, IgG1, IgG2a, IgG2b, and IgG3 Abs in serum were collected at week 11 and levels determined by ELISA. Asterisk(s) indicate statistically significant differences, i.e., p<0.05 (*), between the 2 groups.

Total fecal IgG and IgA levels were determined to correlate changes in intestinal Abs during CD. As shown in FIG. 6, IgA Ab levels in fecal extracts was relatively constant. A significant decline in fecal IgG Abs was observed in IL-10^{-/-} mice that received the IP-10 Ab solution (FIG. 6). These results indicate that blockade of IP-10 attenuated the excretion of IgG Abs from the periphery to the lumen of the intestinal mucosa during murine CD. In addition, total IgG1, IgG2a, IgG2b, IgG3, and IgM antibody levels were compared between the sera of control mice and those treated with anti-CXCL10 Abs. Control and CXCL10 Ab-treated mice had similar levels of IgM, IgG1, IgG2b, and IgG3 Abs. However, total serum IgG2a levels were significantly higher in mice with active colitis, as compared with anti-CXCL10 Ab-treated mice (FIG. 6). The results indicate that blockade of CXCL10 attenuated total IgG2a levels and the excretion of IgG Abs during CD, consistent with the predicted imbalance of Th1>>Th2 cytokine levels during CD.

### Example 8

### Serum IL-12, IFN-γ, IL-2, TNF-α, IL-1α, and IL-1β Levels in IL-10^{-/-} Mice with IBD

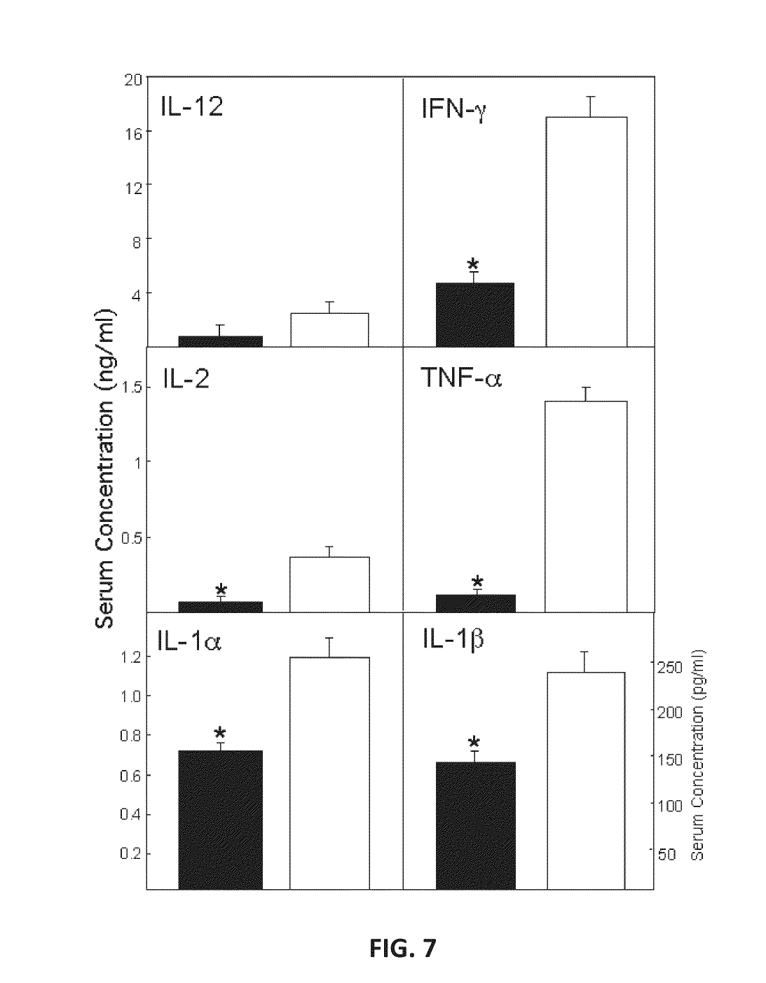

FIG. 7 shows serum IL-12, IFN-γ, IL-2, TNF-α, IL-1α, and IL-1β levels in IL-10^{-/-} mice with IBD. IL-10^{-/-} mice received 200 μl of either pre-immune—(open squares) or anti-mouse IP-10—(closed squares) Ab solutions every 3 days. Serum cytokines, at week 11, levels were determined by ELISA. The data presented are the mean cytokine concentrations±SEM (ng/ml).

Control groups showed moderately higher levels of serum IL-12 p40, compared with IP-10 Ab-treated mice (FIG. 7). In contrast, anti-CXCL10 Ab therapy dramatically decreased IFN-γ levels in IL-10^{-/-} mice, as well as the levels of IL-2, TNF-α, IL-1α, and IL-1β levels. Overproduction of IL-2, IL-12, TNF-α, IL-1α, and IL-1β during IBD has been well documented. The significant decreases in serum IL-2, TNF-α, IL-1α, and IL-1β levels by CXCL10 blockade (FIG. 7) is consistent with the inflammatory state of the host with active colitis being significantly reduced by anti-CXCL10 Ab treatment.

### Example 9

### Histological Characteristics of Colitis Presented by IL-10^{-/-} Mice

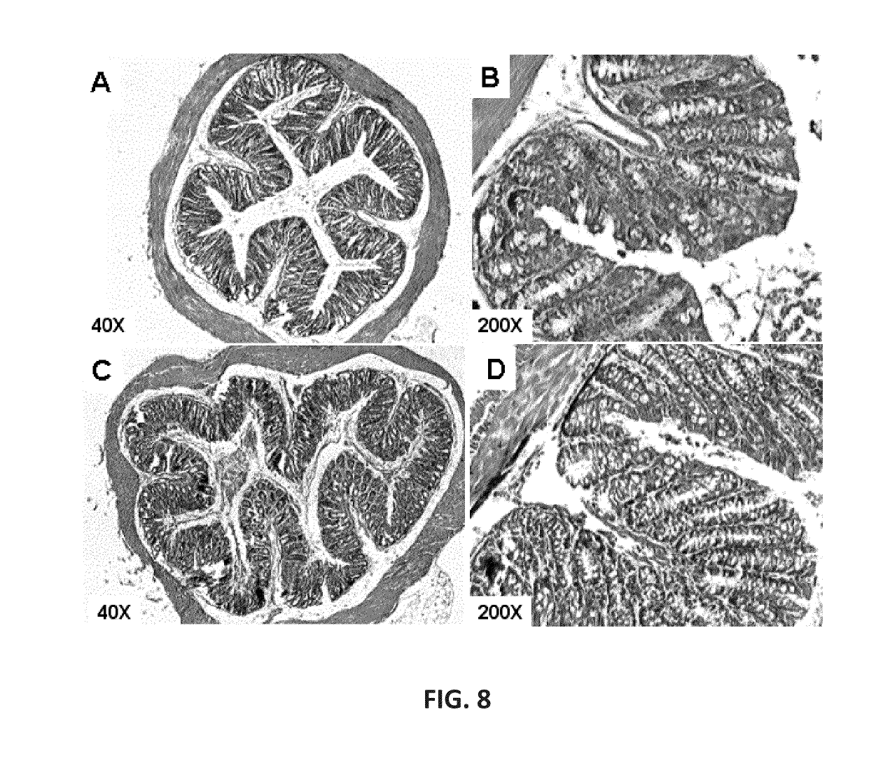

FIG. 8 shows histological characteristics of colitis presented by IL-10^{-/-} mice. Changes in mice that received 200 μl of either pre-immune—(C or D) or anti-mouse IP-10—(A or B) Ab solutions every 3 days. Following sacrifice at week 11, the intestines were fixed, sectioned at 6 μm, and stained. Sections were examined microscopically at a magnification view of 40× (A and C) or 200× (B and D).

Observed pathologic changes included small multifocal infiltrates in the lamina propria of the ascending and transverse colon. These infiltrates consisted of lymphocytes and occasional small numbers of neutrophils. Epithelial cells were not hypertrophied in the IP-10-inhibited group. Multinucleated, enlarged epithelial, and elongated glandular cells were also present in control mice. However, colitis progression was more aggressive in control groups, as noted by multifocal lesions in all regions of the large intestine, especially in colon. The results show a marked improvement in colitis associated with CXCL10 blockade.

### Example 10

### Anti-CXCL10 Antibody Abrogates Severe Colitis

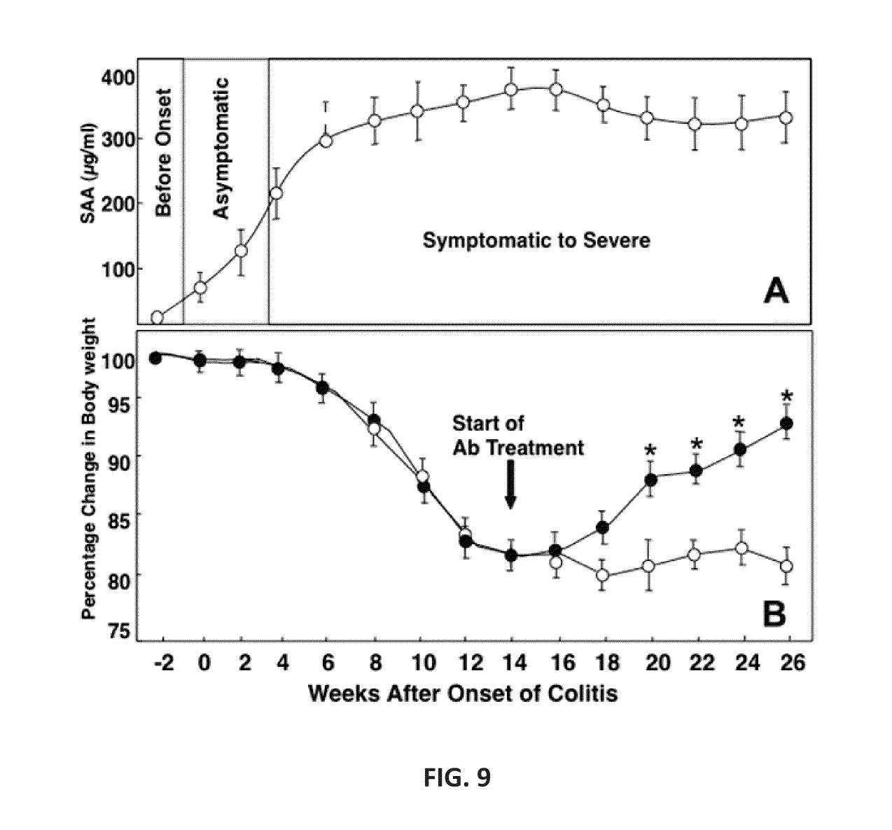

FIG. 9 shows that anti-CXCL10 antibody abrogates severe colitis. IL-10^{-/-} mice received 200 μl of control Ab (open circles) or anti-mouse CXCL10 Ab (closed circles) every 3 days starting 14 weeks after the onset of symptomatic colitis, when mice had lost about 10 to 15% of their initial body weight and attained a peak in SAA levels, and continued until the mice were sacrificed at week 26. The level of SAA±SEM and body weight of the IL-10^{-/-} mice were recorded every week, and the change from initial body weight was expressed as a percentage of the weight before the onset of colitis (week −2) minus the weight at subsequent weeks divided by the weight before the onset of colitis (±SEM). Data represents the mean of three independent experiments involving 5 mice per groups. Asterisks indicate statistically significant differences (p<0.01) between anti-CXCL10 Ab- and control Ab-treated groups.

Chronic colitis in the IL-10 corresponded with an increase in SAA levels (>300 μg/mL)(FIG. 9A) and with a 10%-15% reduction in the body weight of the mice compared with their initial body weight (FIG. 9B). CXCL10 blockade in mice with chronic colitis alleviated weight loss when compared with the weight loss experienced by IL-10^{-/-} mice with chronic colitis treated with control Ab.

### Example 11

### Th1 Cytokine, CXCL10 and CXCR3 mRNA Expression in Mucosal Tissue During Severe Colitis

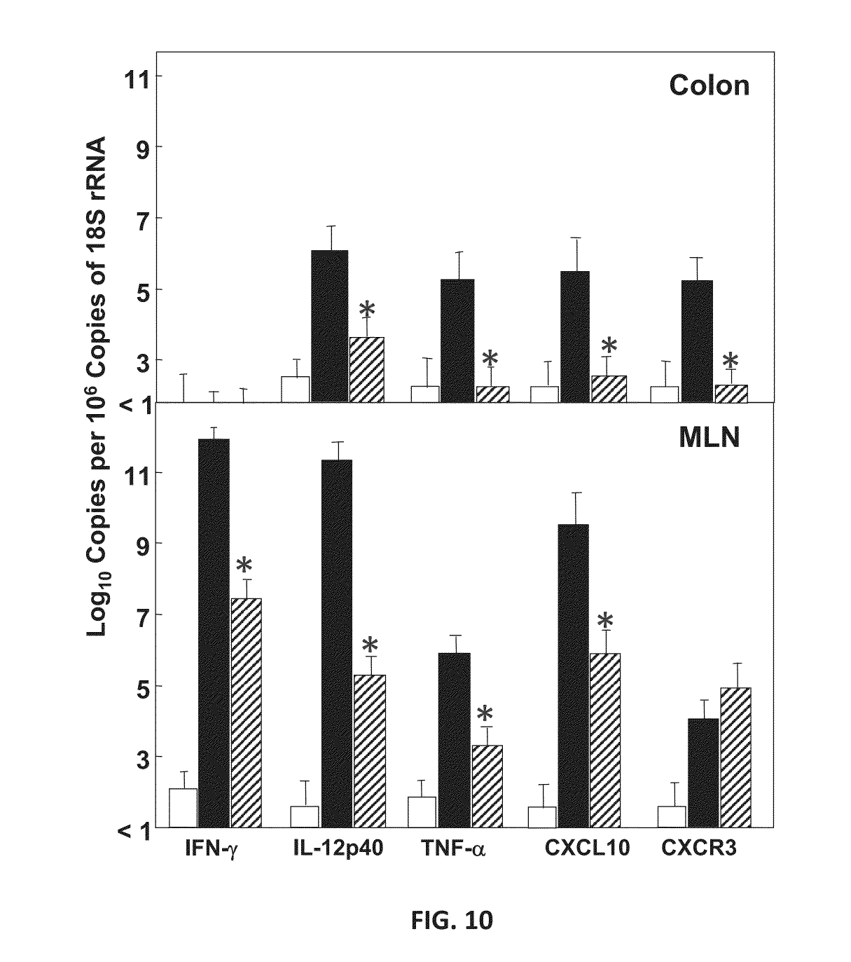

FIG. 10 shows Th1 cytokine, CXCL10 and CXCR3 mRNA expression in mucosal tissue during severe colitis. After chronic development of colitis, mice received 200 μl of either control Ab (solid bars), or anti-CXCL10 Ab (hashed bars) or normal WT mice (open bars), every 3 days starting 14 weeks after the onset of symptomatic colitis, when mice had lost about 15% of their initial body weight. Following sacrifice of the mice, total RNA was isolated from the colons and mesenteric lymph nodes (MLNs) of mice treated with either control Ab, wild type or anti-CXCL10 Ab. The levels of IFN-γ, CXCL10, TNF-α, IL-12p40, and CXCR3 mRNA expression were ascertained by an RT-PCR analysis capable of detecting >20 copies of transcribed cDNA. Log₁₀ copies of transcripts are expressed relative to actual copies of 18S rRNA±SEM in FIG. 10. Data represents the mean of three independent experiments involving 5 mice per group. Asterisks indicate statistically significant differences (p<0.01) between anti-CXCL10 and control Ab-treated groups.

As shown in FIG. 10, significant increases in the expression of TNF-α and IL-12p40 mRNA were noted in the MLNs and colons of IL-10^{-/-} mice with chronic colitis compared with anti-CXCL10 Ab-treated mice. CXCL10 mRNA expression by the colon and MLNs was also significantly elevated during chronic colitis in IL-10^{-/-} mice treated with control Ab compared with anti-CXCL10 Ab-treated mice. IFN-γ Levels were reduced in the MLNs of mice with severe colitis following anti-CXCL10 Ab treatment compared with control Ab treatment; however, this Th1-associated cytokine was below detectable levels in the colons of both groups. CXCR3 mRNA expression was significantly reduced in the colons of IL-10 with colitis after CXCL10 inhibition, but its level in MLNs was not diminished during the same treatment compared with control Ab-treated mice.

### Example 12

### Th1 and Inflammatory Cytokine Levels in Serum During Severe Colitis Progression

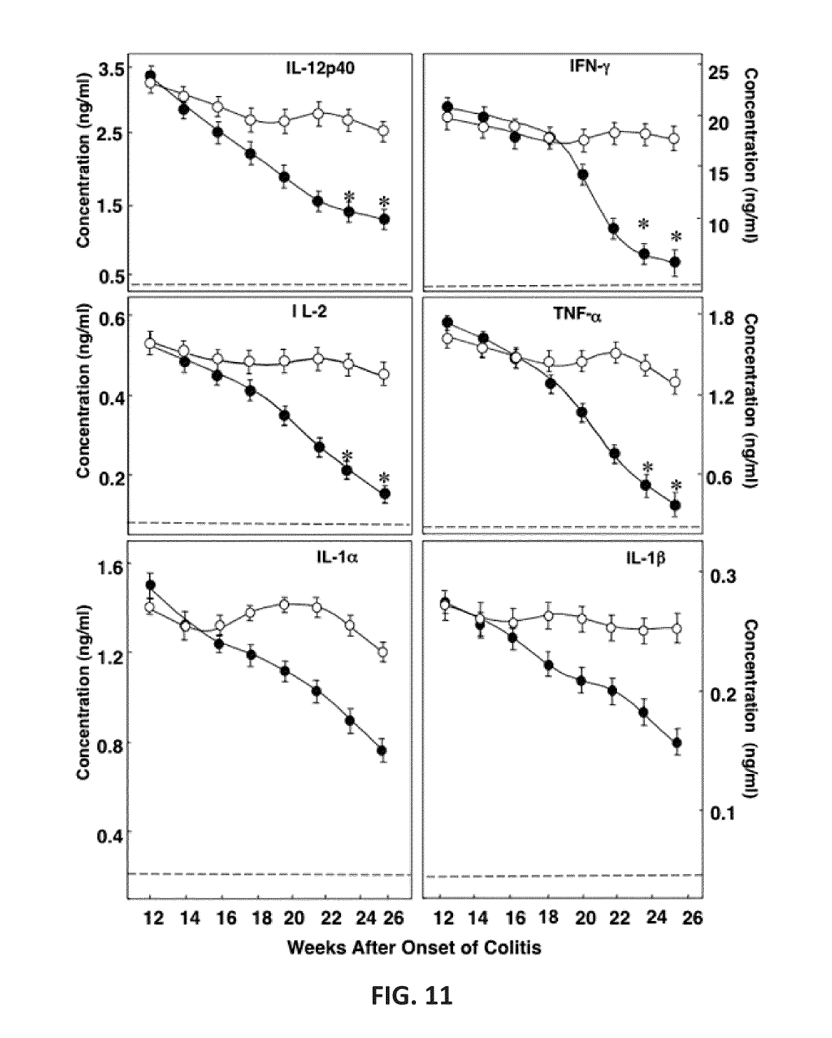

FIG. 11 shows Th1 and inflammatory cytokine levels in serum during severe colitis progression. IL-10^{-/-} mice, received 200 μl of either control Ab (open circles) or anti-CXCL10 Ab (closed circles) every 3 days, starting 14 weeks after the onset of symptomatic colitis, which continued through week 26. Before sacrifice, levels of serum cytokines at week 26 were determined by an ELISA capable of detecting >10 pg/ml of IL-12p40, IL-2, TNF-α, IFN-γ, IL-1α, and IL-1β. The data presented are the mean concentrations±SEM. Asterisk (s) indicate statistically significant differences, i.e., p<0.01 (*), between the two groups. Experimental groups consisted of 5 mice, and experiments were repeated 3 times. Data represents the mean of 3 independent experiments.

Consistent with the RT-PCR analysis in FIG. 10, anti-CXCL10 Ab treatment decreased IFN-γ and IL-12p40 serum levels in IL-10 with chronic colitis (FIG. 11). Serum IL-2, TNF-α, IL-1α, and IL-1β levels also declined after CXCL10 blockade in IL-10^{-/-} mice with chronic colitis compared with the control Ab-treated mice. These data indicate that CXCL10 blockade caused the reduction of SAA, IL-6, IL-12p40, IFN-γ, IL-2, TNF-α, IL-1α, and IL-1β serum levels of the IL-10^{-/-} mice with chronic colitis.

### Example 13

### Anti-CXCL10 Antibody Effects on Colitis Pathology

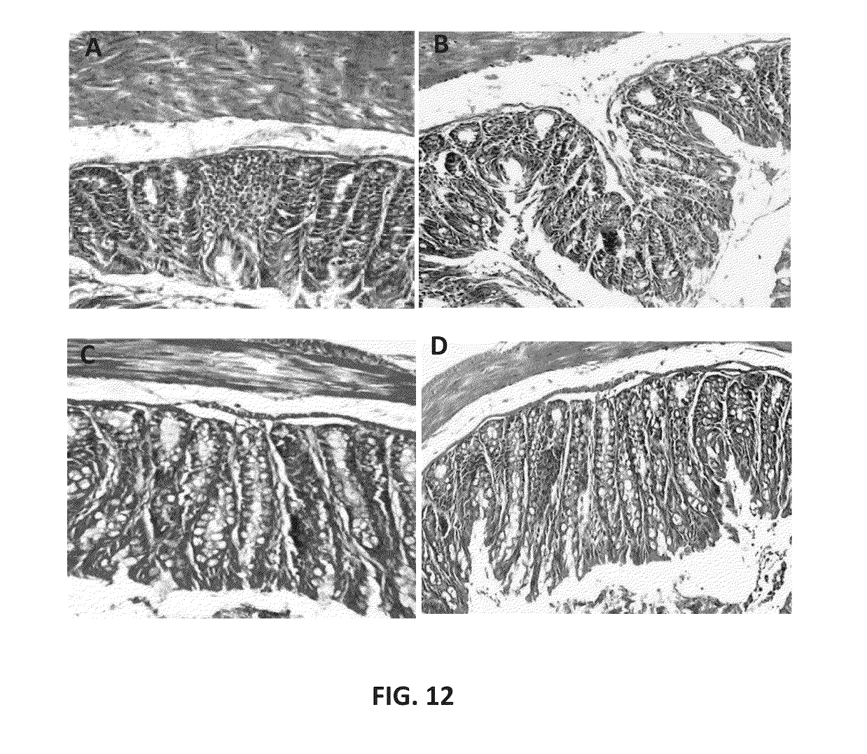

FIG. 12 shows anti-CXCL10 antibody effects on colitis pathology. Histopathology of the colons from IL-10^{-/-} mice with chronic colitis that were treated with either control Ab, (Panels A and B) or anti-CXCL10 Ab (panels C-D) as before. Sections were examined by light microscopy. Experimental groups consisted of 5 mice and were repeated 3 times.

The mice that received anti-CXCL10 Ab showed a significant reduction in intestinal inflammation. An increase in leukocyte infiltrates (FIG. 12A) and distortion of glandular architecture (FIG. 12B) were observed in the intestines during chronic colitis. Anti-CXCL10 Ab reduced the lymphocyte infiltration and partially restored the glandular and goblet cell architecture (FIG. 12C), which also coincided with lengthening of intestinal crypts FIG. 12D). In addition, the mean histologic scores of IL-10^{-/-} mice with severe colitis that received control Ab were significantly higher than the scores of mice treated with anti-CXCL10 Ab (data not shown). Similarly, SAA levels correlated with the severity of colitis as determined by histologic analysis. Pathologic changes included leukocyte infiltrates in the LP of the colon of control Ab-treated IL-10 with the number of these infiltrates being reduced after CXCL10 blockade. Taken together, the results show a marked improvement in the characteristic intestinal inflammation associated with chronic colitis after CXCL10 blockade.

### Example 14

### Histological and Immunofluorescence Localization of CXCL9, CXCL10, CXCL11, and TNF-α in the Colon of CD Patients

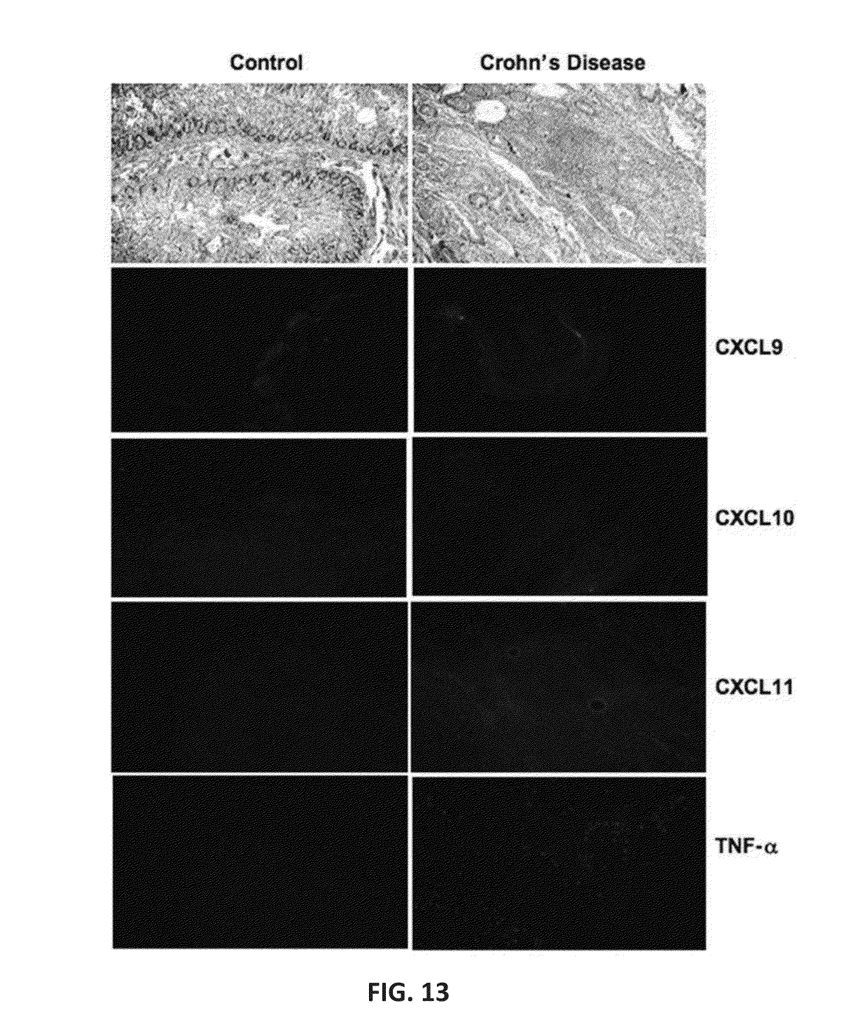

FIG. 13 shows histological and immunofluorescence localization of CXCL9, CXCL10, CXCL11, and TNF-α in the colon of CD patients. Histopathology of colonic changes in the intestines of CD patients and normal control were fixed, sectioned at 6 μm, and stained with hematoxylin and eosin or anti-CXCL9, CXCL10, CXCL11 or TNF-α antibodies. Sections were examined at a magnification view of 130×. The inflamed colon demonstrates the differences in mucosal wall thickness, crypt malformation, leukocyte infiltration, and glandular elongation between normal and CD patients.

The colon pathology of control samples showed hypertrophied epithelial layers at multiple sites, with only a few inflammatory infiltrates and low expression of CXCL9, CXCL10, CXCL11 and CXCR3 (FIG. 13). In contrast, CD patients with high levels of serum CXCL9, CXCL10, and CXCL11 also expressed significant levels of CXCL11>>CXCL9 with modest increases in CXCl10 in the colon.

### Example 15

### MAP-Specific Serum Ab Responses in IL-10^{-/-} Mice During Spontaneous Colitis

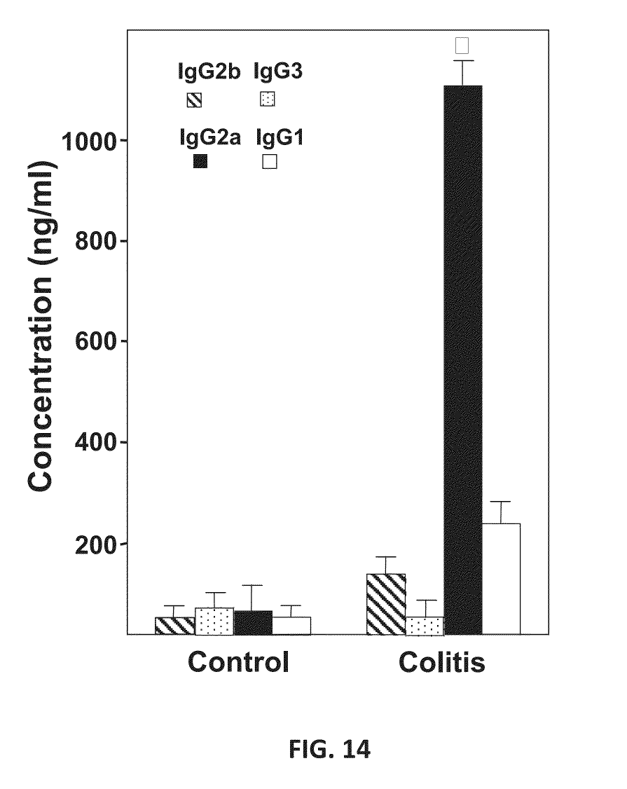

FIG. 14 shows M. avium subsp. paratuberculosis (MAP)-specific serum Ab responses in IL-10^{-/-} mice during spontaneous colitis. The data presented are the mean±SD concentration (ng/ml) of MAP-specific IgG subclasses from three separate experiments. Asterisks (*) indicate statistically significant differences, i.e., p<0.01, compared to controls. Mice experimental groups consisted of 15 mice. Assays were repeated 3 times.

FIG. 14 shows that MAP-specific IgG2a Ab responses were significantly higher in mice with spontaneous colitis, kept under conventional housing, than in similar control mice without disease, which were housed under germfree conditions. This is consistent with the previously described imbalance of cytokine levels (Th1>Th2) during colitis, suggesting there is a Th1-biased humoral response associated with the progression of colitis.

### Example 16

### Histological Characteristics of IL-10^{-/-} Mice Challenged with MAP

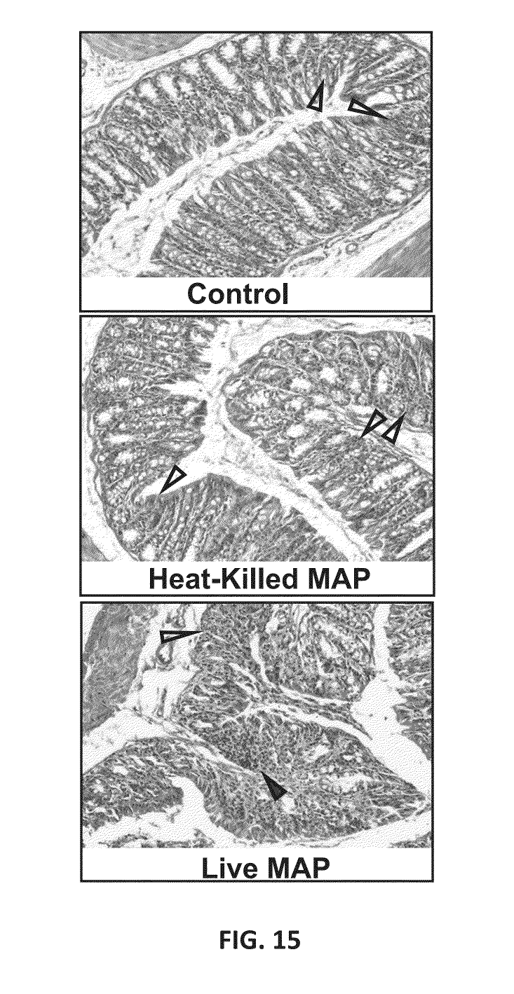

FIG. 15 shows histological characteristics of IL-10^{-/-} mice challenged with MAP. 14 weeks post challenge, histopathologies of colons from IL-10^{-/-} mice that received a single dose of 200 μl of control vehicle (cream only), 10⁴ CFU of live MAP in cream, or 10⁴ CFU of heat-killed MAP in cream by gavage and maintained under otherwise germ-free conditions were fixed, sectioned at 6 μm, and stained with hematoxylin and eosin. Mild (open triangles) and heavy (solid triangles) cellular infiltrates were noted in groups (i.e., live MAP>>heat-killed MAP>controls). In live MAP challenged mice, aggregates of cellular infiltrates were typically associated with focal lesions and hypertrophied epithelial cells with reduced crypt lengths. Sections were examined by light microscopy (40× magnification). Experimental groups consisted of 15 mice. Representative samples are shown.

FIG. 15 shows that the intestinal tissues of mice challenged with live M. avium subsp. paratuberculosis exhibited increased levels of cellular infiltrates, which consisted of lymphocytes and, occasionally, polymorphonuclear cells. The colitis progression was more aggressive in mice that received live M. avian subsp. paratuberculosis, as noted by multifocal lesions, or aggregates of cellular infiltrates, in all regions of their large intestines. In addition, epithelial cells in mice challenged with live M. avium subsp. paratuberculosis were hypertrophied, the intestinal crypt length was decreased, and elongated glandular cells were also present in both the mucosa and the submucosa.

### Example 17

### Changes in Body Weight of IL-10^{-/-} Mice after MAP Challenge

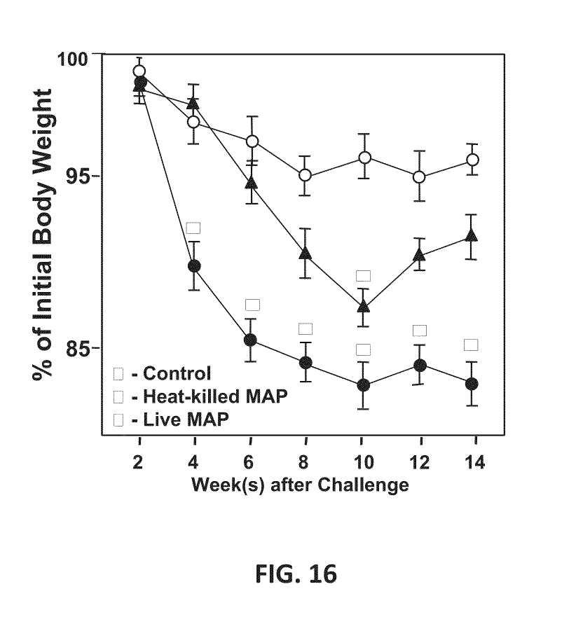

FIG. 16 shows changes in body weight of IL-10^{-/-} mice after MAP challenge. The wasting disease associated with murine colitis was observed by monitoring the body weight during colitis progression. IL-10^{-/-} mice on B6 background, received a single dose of 200 μl normal control (cream, open circles), 10⁴ CFUs of live MAP in cream (solid circles) or 10⁴ CFUs of pasteurized MAP in cream (triangles) and maintained under otherwise germ-free conditions. Percentage of initial body weight of IL-10^{-/-} mice was recorded biweekly. The data presented are the mean±SD of 3 separate experiments. Asterisks (*) indicate statistically significant differences, i.e., p<0.01, compared to controls. Experimental groups consisted of 15 mice and assays were repeated 3 times.

FIG. 16 shows that mice challenged with M. avium subsp. paratuberculosis and housed under otherwise germfree conditions lost more body weight and experienced higher SAA levels than did similar mice challenged with heat-killed M. avium subsp. paratuberculosis or given the control vehicle. Mice exposed to heat-killed M. avium subsp. paratuberculosis had less weight loss than those exposed to live M. avium subsp. paratuberculosis but had only a marginal increase in the SAA level. The results indicate that mice challenged with live M. avium subsp. paratuberculosis show rapid colitis progression associated with elevated SAA levels and reductions in body weight compared with the control group.

### Example 18

### Serum Cytokine Levels in IL-10^{-/-} Mice after MAP Challenge

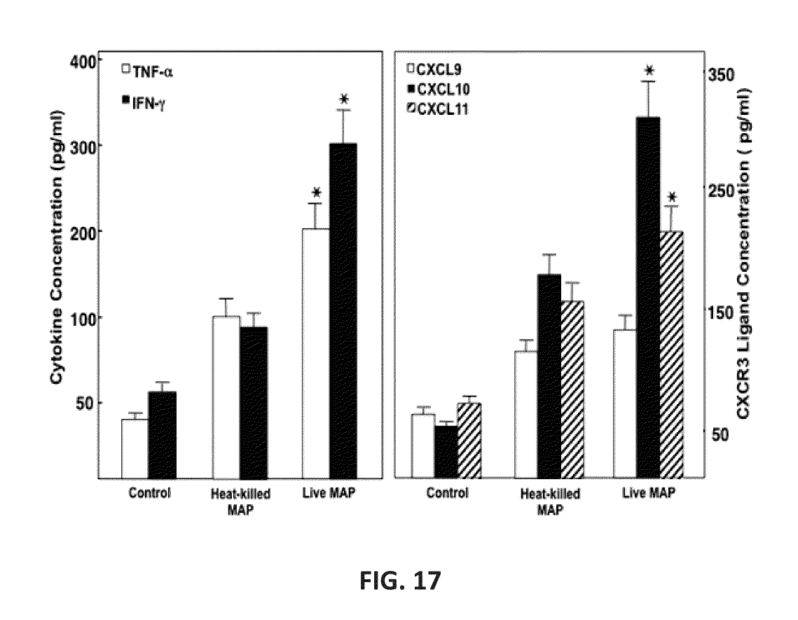

FIG. 17 shows serum cytokine levels in IL-10^{-/-} mice after MAP challenge. IL-10^{-/-} mice, on a B6 background, received a single dose of 200 μl of the control vehicle (i.e., cream), 10⁴ CFUs of live MAP in cream, or 10⁴ CFUs heat-killed MAP in cream by gavage and maintained under otherwise germ-free conditions. The levels of serum TNF-α and IFN-γ and CXCL9, CXCL10, and CXCL11 14 weeks after challenge were determined by ELISA, capable of detecting >10 pg/ml TNF-α, IFN-γ or CXCR3 ligand. The data presented are the mean TNF-α, IFN-γ, and CXCR3 ligand concentrations±SD (ng/ml). Asterisks (*) indicate statistically significant differences, i.e., p<0.01, compared to controls. Experimental groups consisted of 15 mice. Assays were repeated 3 times.

Following M. avium subsp. paratuberculosis challenge, IFN-γ and TNF-α levels were significantly higher (˜6-fold) in sera of IL-10^{-/-} challenged with live M. avium subsp. paratuberculosis than in control mice; mice exposed to heat-killed M. avium subsp. paratuberculosis had ˜2-fold greater TNF-α and IFN-γ responses than those of controls, but these differences were not significant (FIG. 17). Serum levels of CXCL10 and CXCL11, but not CXCL9, were significantly increased in mice challenged with live or heat-killed M. avium subsp. paratuberculosis compared with those for mice in the control group. These results indicate that exposure to M. avium subsp. paratuberculosis increased the production of systemic IFN-γ, TNF-α, CXCL10, and CXCL11.

### Example 19

### Anti-Peptide #25 Ag (from MPT59)-Induced Proliferation and IL-2 Production by CD4⁺ T Cells from IL-10^{-/-} Mice

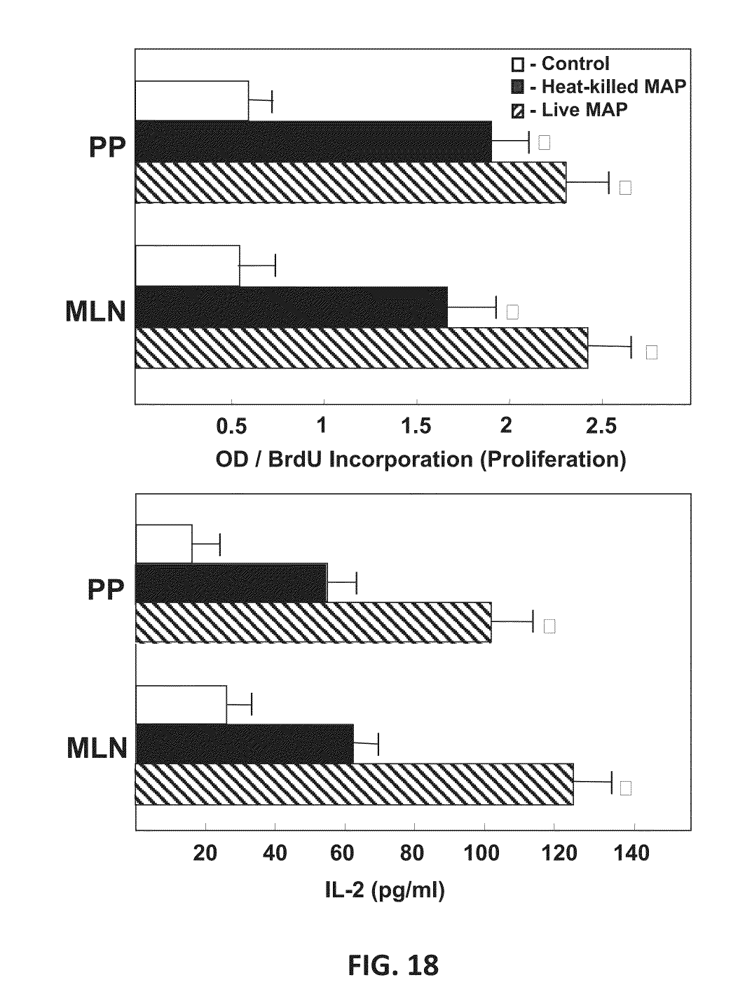

FIG. 18 shows anti-peptide #25 Ag (from MPT59)-induced proliferation and IL-2 production by CD4⁺ T cells from IL-10^{-/-} mice. IL-10^{-/-} mice, on B6 background, received a single dose of 200 μl of control vehicle (open bars, cream only), 10⁴ CFUs of live MAP in cream (hatched bars), or 10⁴ CFUs of heat-killed MAP in cream (solid bars) and maintained under otherwise germ-free conditions. CD4′ lymphocytes derived from the MLN, and PPs of the mice were purified and cultured at density of 5×10⁶ cells/ml with peptide #25 (1 μg/ml) for 3 days with γ-irradiated APCs (10⁶ cells/ml). Cytokines present in culture supernatants were determined ELISA. Proliferation was measured by BrdU incorporation. The data presented are the mean OD₄₅₀ for proliferative responses or the mean of IL-2 secretion (μg/ml)±SD of quadruplicate cultures. Asterisks (*) indicate statistically significant differences, i.e., p<0.01, compared to controls. Experimental groups consisted of 15 mice and experiments were repeated three times.

FIG. 18 shows that peptide 25-stimulated CD4⁺ T cells from the MLN and PP of mice previously challenged with either live or heat-killed M. avium subsp. paratuberculosis exhibited marked increases in BrdU incorporation compared with similar CD4⁺ T cells from mice challenged with cream alone. These results suggest that Ag restimulation after exposure to M. avium subsp. paratuberculosis enhances CD4⁺ T-cell proliferation.

### Example 20

### Serum CXCR3 Ligands and Mycobacterial-Specific Ab Responses in IBD Patients

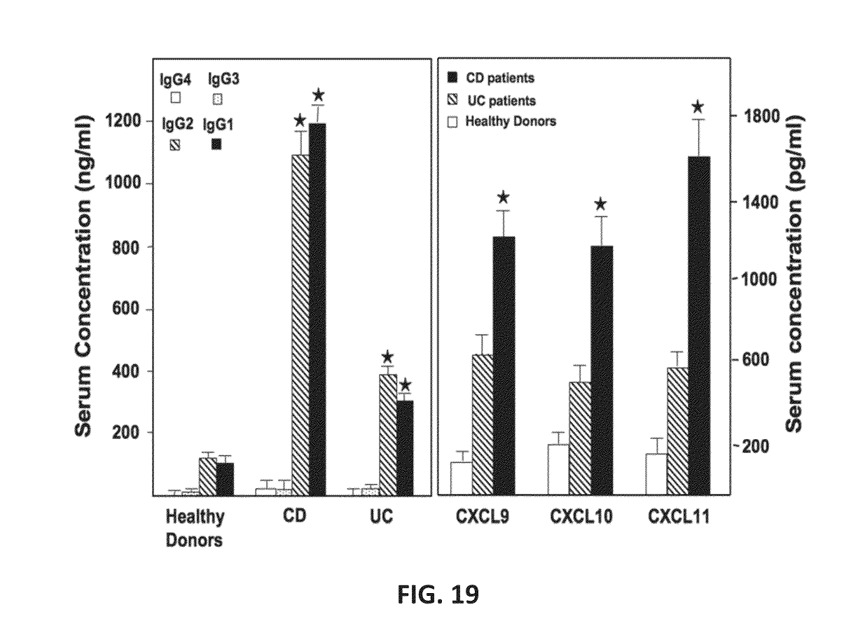

FIG. 19 shows serum CXCR3 ligands and mycobacterial-specific Ab responses in IBD patients. Sera from 62 CD and 88 UC female patients and 32 normal healthy female donors, not undergoing any treatment, were isolated and evaluated for the presence of CXCR3 ligands (i.e., CXCL9, CXCL10, and CXCL11) and mycobacterial-specific IgG1, IgG2, IgG3 and IgG4 Abs. These levels were determined by ELISAs capable of detecting 10>pg/ml of these ligands. The data presented are concentrations±SEM. Asterisk(s) indicate statistically significant differences, i.e., p<0.01, between healthy donors and IBD patients.

While total IgG1, IgG2, IgG3, and IgG4 subclass Abs were significantly higher in the sera of IBD patients compared to healthy donors (data not shown), the profile of the IgG humoral response in IBD patients also revealed increases in Mycobacteria-specific IgG1 and IgG2 Abs (FIG. 19). These responses in CD patients were significantly higher than in UC patients or normal healthy donors. CXCR3 ligands were also increased in these samples than compared to healthy donors. These results suggest that IBD patients have higher CXCL9, CXCL10, and CXCL11 levels and Mycobacteria-specific IgG1 and IgG2 Ab responses. Moreover, these findings correlate with previous findings showing higher levels of Mycobacteria-specific IgG2a and CXCR3 ligands during spontaneous colitis in IL-10^{-/-} mice under conventional housing.

### Example 21

### Changes in SAA Levels in IBD Patients and in IL-10^{-/-} Mice after Mycobacterial Challenge

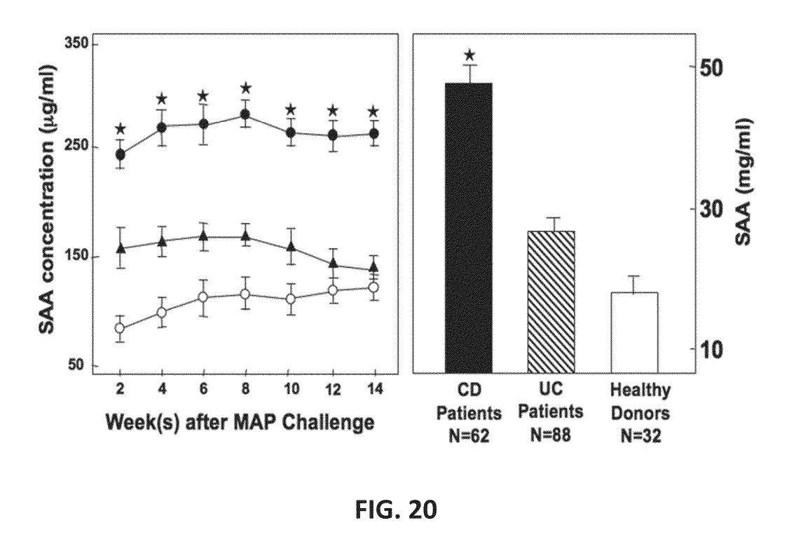

FIG. 20 shows changes in SAA levels in IBD patients and in IL-10^{-/-} mice after mycobacterial challenge. IL-10^{-/-} mice on B6 background, received 200 μl of cream milk alone (open circles; control) or cream milk containing 10⁴ CFU of live (closed circles) or heat-killed (closed triangles) M. avium paratuberculosis. SAA levels during Mycobacteria-enhanced colitis as well as IBD patients and healthy donors were measured by ELISA. Experimental groups consisted of 5 mice, and experiments were repeated 3 times. The data presented are the mean±SEM concentration of SAA. Asterisks indicate statistically significant differences, i.e., p<0.01, between control and Mycobacteria-treated groups or healthy donors and IBD patients.

The results in FIG. 20 show that mice challenged with live Mycobacteria in otherwise specific pathogen-free conditions experienced a significant rise in SAA levels when compared to similar mice challenged with heat-killed Mycobacteria or control mice.

### Example 22

### Intestinal Histological Characteristics of IL-10^{-/-} Mice Challenged with Mycobacteria

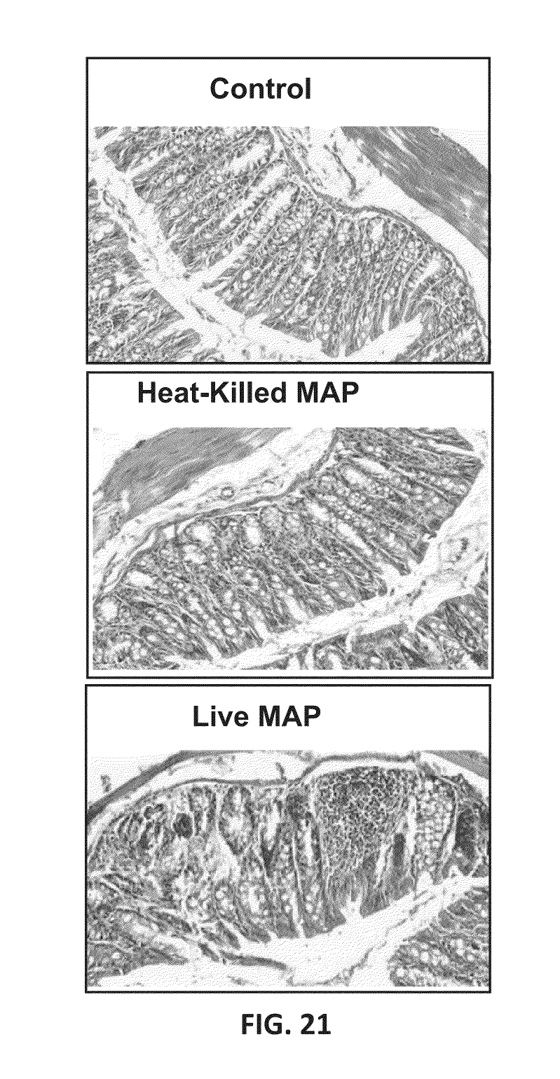

FIG. 21 shows intestinal histological characteristics of IL-10^{-/-} mice challenged with Mycobacteria. IL-10^{-/-} mice on B6 background, received 200 μl of cream milk alone ((open circles; control) or cream milk containing 10⁴ CFU of live (closed circles) or heat-killed (closed triangles) M. avium paratuberculosis. After sacrifice, intestines were fixed, sectioned at 6 μm, and stained with hematoxylin and eosin. Sections were examined by light microscopy. Experimental groups consisted of 5 mice and experiments were repeated 3 times.

The intestinal tissues of mice challenged with Mycobacteria showed higher increases in leukocyte infiltrates, which consisted of lymphocytes and occasionally polymorphonuclear cells as well as a higher frequency of lymphoid follicles in live versus heat-killed Mycobacteria-challenged groups (FIG. 21). Moreover, colitis was more aggressive in mice that received live Mycobacteria, as noted by multi-focal lesions and aggregates of leukocyte infiltrates in the large intestines, than compared to control mice.

### Example 23

### Serum CXCL9, CXCL10 and CXCL11 Concentrations in IC Patients

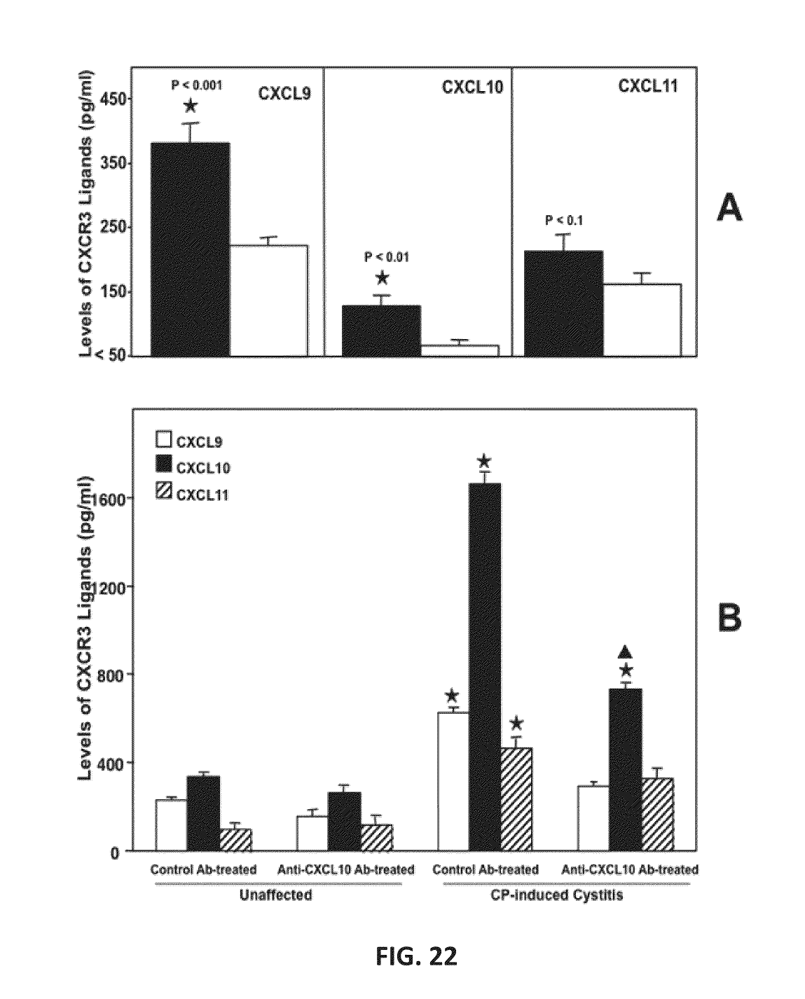

FIG. 22 shows serum CXCL9, CXCL10 and CXCL11 concentrations in IC patients. Panel A: Sera from IC patients (n=32) and normal, healthy donors (n=16) were isolated and evaluated for the presence of CXCR3 ligands by ELISA, capable of detecting >10 pg/ml of each CXCR3 ligand. The data presented are the mean CXCL9, CXCL10, and CXCL11 of IC patient and normal healthy donors concentrations±SEM. Asterisks (*) indicate statistically significant differences, i.e., p<0.01, between the healthy donors and IC patients. Panel B: Control or anti-CXCL10 Ab solutions were administered 2 days prior to CYP challenge and every 2 days thereafter. Five days after CYP administration, the serum levels of CXCL9, CXCL10, and CXCL11 were determined by ELISA. The data presented are the mean concentrations±SEM in each group. Asterisks (*) indicate statistically significant (p<0.01) differences between unaffected and CYP-induced groups. Triangles indicate statistically significant (p<0.01) differences between control Ab- and anti-CXCL10 Ab-treated groups administered CYP.

As shown in FIG. 22A, the serum levels of CXCL9 and CXCL10 in IC patients were significantly higher than levels in unaffected healthy donors. In particular, the difference in serum levels between IC patients and healthy donors were greatest for CXCL9 (p<0.001), followed by CXCL10 (p<0.01) and CXCL11 (p>0.1). These CXCR3 ligand levels also correlated (although not statistically significant) with disease severity as manifested by pathological reports for each individual patient (data not shown). Further, these patients showed multiple pathological features of tissue damage that frequently included urothelium denudation, mucosal edema, and/or leukocyte infiltration.

CYP-induced cystitis in mice led to substantial increases in serum levels of CXCL10>>CXCL9 when compared with the levels in unaffected controls (FIG. 22B). In confirmation with serum CXCR3 ligand levels in IC patients, murine CXCL11 levels did not significantly change in groups induced with CYP. In summary, mice with CYP-induced cystitis expressed higher serum CXCL10>CXCL9 than unaffected controls, while IC patients displayed higher CXCL9>CXCL10 serum levels than unaffected individuals.

### Example 24

### Histological Changes after CYP-Induced Cystitis

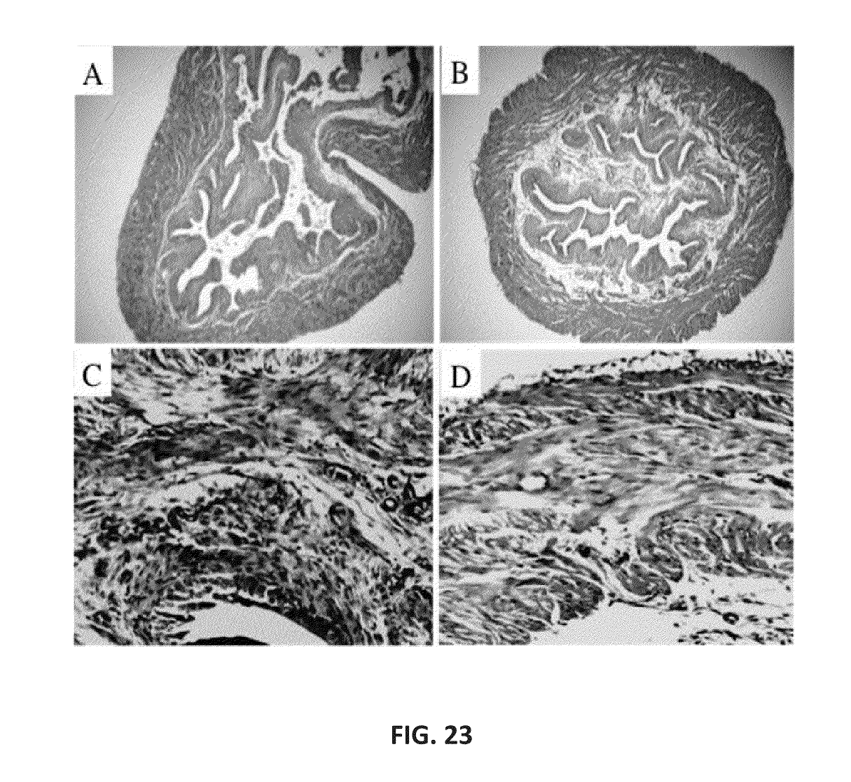

FIG. 23 shows histological changes after CYP-induced cystitis. Control or anti-mouse CXCL10 Ab solutions were administered 2 days prior to CYP treatment and every 2 days thereafter. Five days after CYP administration, the urinary bladders of the mice were fixed, sectioned at 6 μm, and stained with hematoxylin and eosin. The sections were examined microscopically at magnification views of 10× and 100×. Panels A and C show the magnified sections from control Ab-treated mice, while Panels B and D display similar sections from anti-CXCL10 Ab-treated mice given CYP to illustrate inflamed bladders and characterized differences in mucosal wall thickness, enlargement of mucosal layer, leukocyte infiltration, and glandular elongation.

Control Ab-treated mice given CYP showed pathological signs of cystitis (i.e., urinary bladder inflammation, discontinuous uroepitheium). However, affected mice treated with anti-CXCL10 Ab displayed a reduction in cystitis, as noted by a decrease in urinary bladder leukocyte infiltrates (FIG. 23). Histological differences between control Ab- and anti-CXCL10 Ab-treated mice with CYP-induced cystitis were considered significant and showed that CXCL10 blockade significantly reduced CYP-induced cystitis.

### Example 25

### CXCR3, -9, -10, and -11 mRNA Expression in CYP-Treated Mice

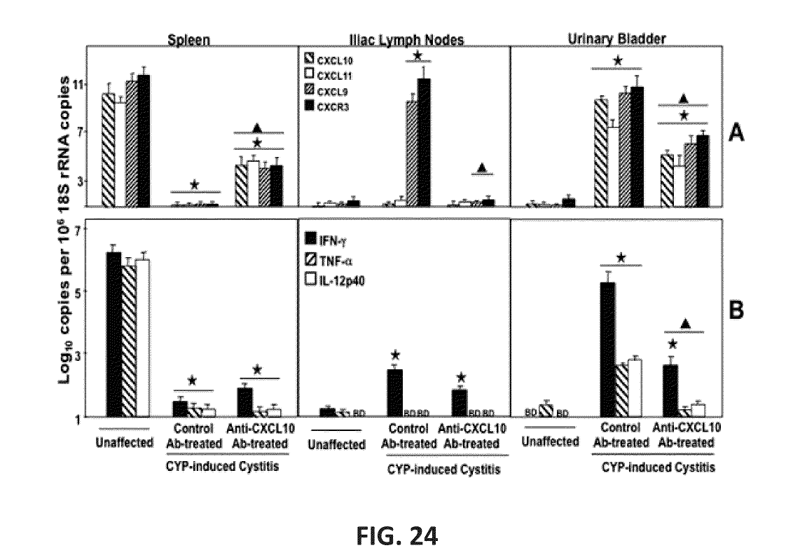

FIG. 24 shows CXCR3, CXCL9, CXCL10, and CXCL11 mRNA expression in CYP-treated mice. Control or anti-mouse CXCL10 Ab solutions were administered 2 days prior to CYP treatment and every 2 days thereafter. Five days after CYP administration, total RNA was isolated from the spleen, iliac lymph nodes, or urinary bladder of the mice. Panel A: RT-PCR analysis of CXCR3, CXCL9, CXCL10, or CXCL11 mRNA expression was performed. Panel B: RT-PCR analysis of IFN-γ, IL-12 p40, or TNF-α mRNA expression was performed. Log₁₀ copies of transcripts±SEM are expressed relative to actual copies of 18S rRNA. Asterisks (*) indicate statistically significant (p<0.01) differences between unaffected and CYP-induced groups. Triangles indicate statistically significant (p<0.01) differences between control Ab- and anti-CXCL10 Ab-treated groups administered CYP.

As shown in FIG. 24A, CYP-induced cystitis in mice led to substantial increases in the expression of CXCL10, CXCL11, and CXCR3 mRNA by urinary bladder leukocytes as well as modest increases in the expression of CXCL9 and CXCR3 transcripts by iliac lymph node lymphocytes than compared to normal, untreated mice. In contrast, the expression of these IFN-γ- and nuclear factor kappa B (NFκB)-inducible chemokines and CXCR3 mRNAs were significantly diminished in splenocytes from CYP-treated mice than compared to similar cells from control mice. Anti-CXCL10 Ab treatment significantly decreased the expression of CXCL9 and CXCR3 mRNAs by iliac lymph node leukocytes and reduced the production of CXCL9, CXCL10, CXCL11, and CXCR3 mRNAs by urinary bladder leukocytes.

To investigate local and peripheral changes in Th1 and inflammatory cytokine expression during CYP-induced cystitis, the levels of IFN-γ, IL-12p40, and TNF-α mRNAs expressed by leukocytes isolated from the spleen, iliac lymph nodes and urinary bladder were measured by quantitative RT-PCR analysis. CYP-induced mice receiving control Ab exhibited substantial decreases in the expression of IFN-γ, IL-12p40, and TNF-α mRNAs by splenocytes; however, this treatment significantly increased the expression of cytokines by urinary bladder leukocytes than compared to unaffected mice (FIG. 24B). Mice with CYP-induced cystitis exhibited increased IFN-γ mRNA expression by iliac lymph node lymphocytes compared to similar cells from unaffected mice. However, the expression of IFN-γ, IL-12p40, and TNF-α mRNAs by urinary bladder lymphocytes from mice with cystitis were significantly decreased following anti-CXCL10 Ab treatment than compared to similar cells from CYP-induced mice treated with control Ab.

### Example 26

### Serum CXCL10 Concentrations During Active Crohn's Disease (CD)

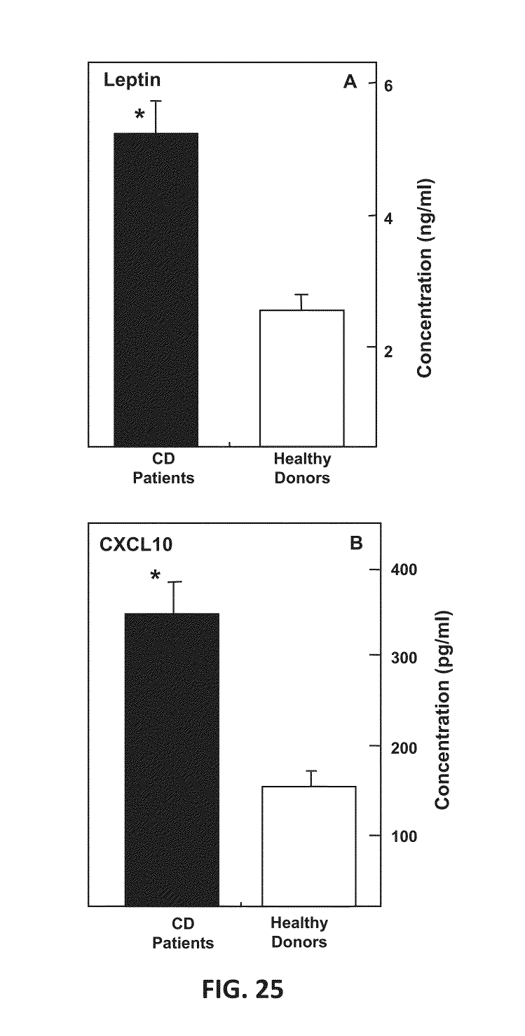

FIG. 25 shows upregulated CXCL10 expression during active CD. Sera from CD patients (n=120) and normal healthy donors (n=30), not undergoing treatment, were isolated and evaluated for the presence of CXCL10. The levels of CXCL10 were determined by an ELISA assay capable of detecting >20 pg/ml of CXCL10. The data presented are the mean CXCL10 concentrations±SEM in CD patients and healthy donors. Asterisk(s) indicate statistically significant differences, i.e., p<0.05 (*), between the 2 groups.

The results in FIG. 25 show that CD patients exhibited significant increases in leptin and CXCL10 compared to healthy donors.

### Example 27

### Serum CXCL11 and CXCL9 Concentrations During Active Crohn's Disease

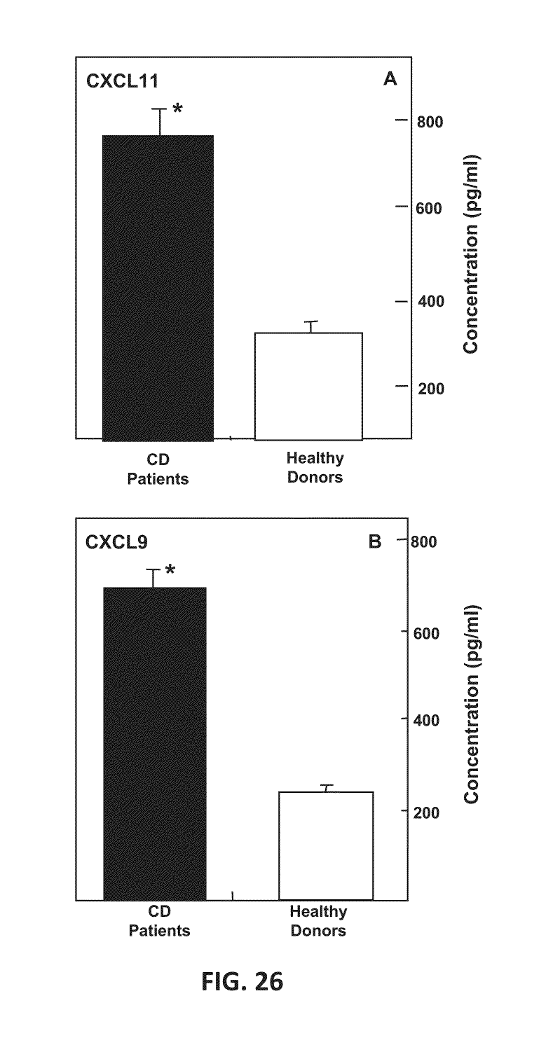

FIG. 26 shows upregulated expression of CXCL11 and CXCL9 during active CD. Sera from CD patients (n=120) and normal healthy donors (n=30), not undergoing treatment, were isolated and evaluated for the presence of CXCL11 and CXCL9. The levels of serum CXCL11 and CXCL9 were determined by ELISA that was capable of detecting >20 pg/ml of each Th1 cytokine. The data presented are mean CXCL11 (FIG. 26A) and CXCL9 (FIG. 26B) concentrations±SEM in CD patients and healthy donors. Asterisk(s) indicate statistically significant differences, i.e., p<0.05 (*), between the 2 groups.

The results in FIG. 26 show that CD patients exhibited significant increases in leptin and CXCL11 and CXCL9 compared to healthy donors.

### Example 28

### Serum Amyloid Protein A (SAA) and IL-6 Concentrations During Active Crohn's Disease

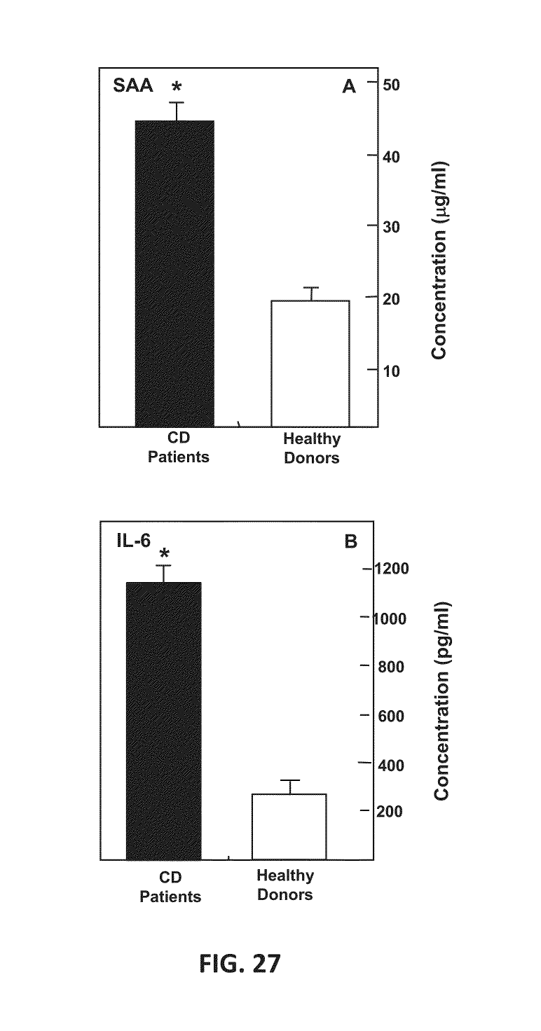

FIG. 27 shows upregulated serum concentrations of serum amyloid A (SAA) and IL-6 in CD patients. Sera from CD patients (n=120) and normal healthy donors (n=30), not undergoing treatment, were isolated and evaluated for the presence of SAA and IL-6 levels. The levels of serum SAA and IL-6 were determined by ELISA that was capable of detecting 20>pg/ml of the SAA and IL-6 concentration. The data presented are the mean of SAA (FIG. 27A) and IL-6 (FIG. 27B) concentrations±SEM in CD patients and healthy donors. Asterisk(s) indicate statistically significant differences, i.e., p<0.05 (*), between the 2 groups. This data is consistent with elevated SAA and serum IL-6 levels corresponding with the severity of CD.

The results in FIG. 27 show that CD patients exhibited significant increases in SAA and IL-6 compared to healthy donors.

### Example 29

### Serum IL-12p40 and IFN-γ Levels Correlate During Active Crohn's Disease

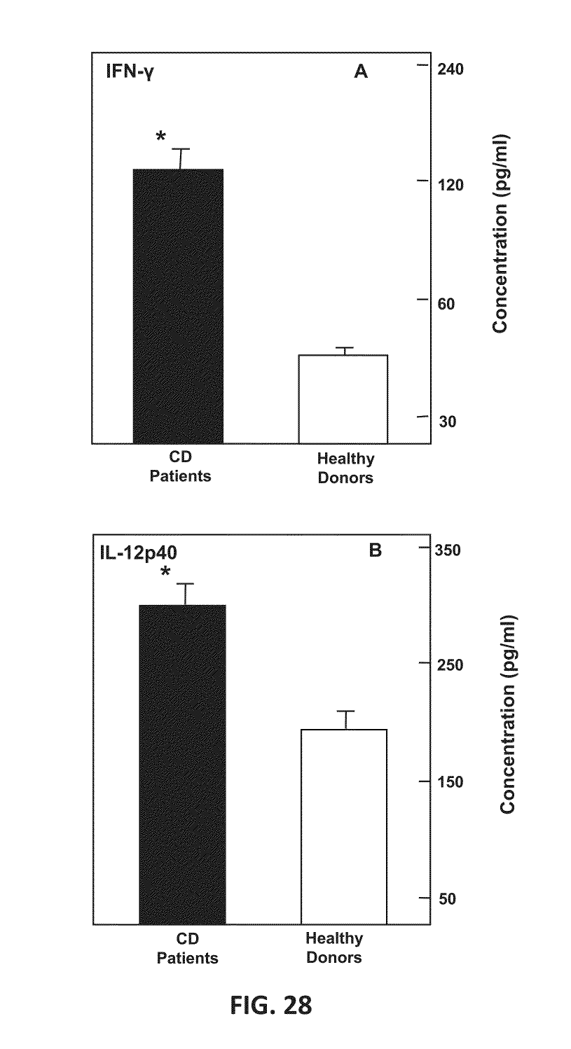

FIG. 28 shows serum IL-12p40 and IFN-γ levels correlate during CD. Sera from CD patients (n=120) and normal healthy donors (n=30), not undergoing treatment, were isolated and evaluated for the presence of IL-12p40 and IFN-γ. The levels of serum IFN-γ and IL-12p40 were determined by ELISA that was capable of detecting >20 pg/ml of each cytokine. The data presented are the mean IL-12p40 (FIG. 28A) and IFN-γ (FIG. 28B) concentrations±SEM from the serum of CD patients and healthy donors. Asterisk(s) indicate statistically significant differences, i.e., _{p<}0.05 (*), between the 2 groups.

The results in FIG. 28 show that CD patients exhibited significant increases in IFN-γ and IL-12p40 compared to healthy donors.

### Example 30

### Inflammatory Cytokine Levels During Active Crohn's Disease

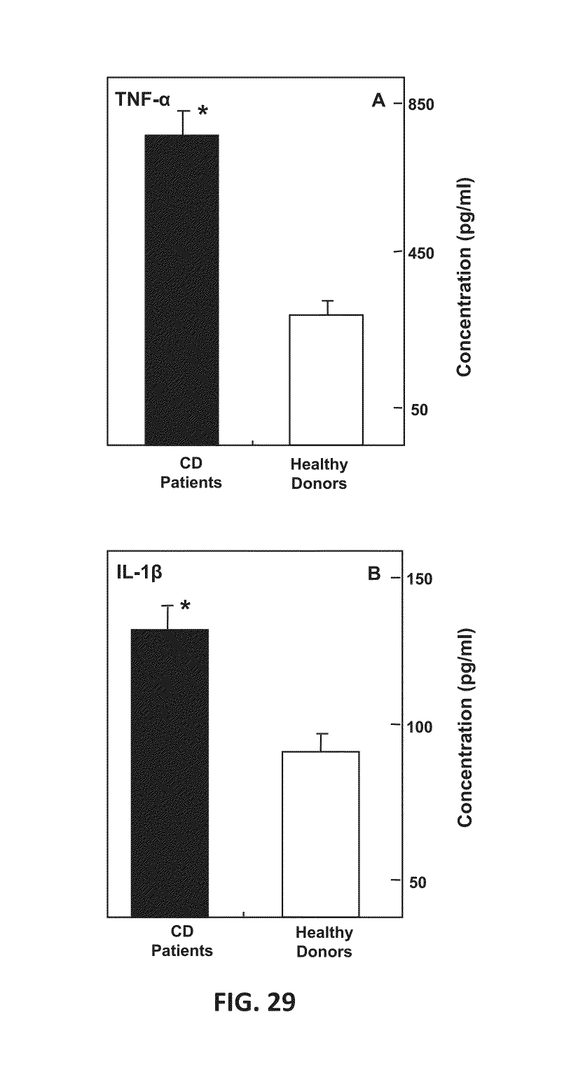

FIG. 29 shows inflammatory cytokine levels during active CD. Sera from CD patients (n=120) and normal healthy donors (n=30), not undergoing treatment were isolated and evaluated for the presence of TNF-α and IL-1β. The levels of serum TNF-α and IL-1β were determined by ELISA that was capable of detecting >20 pg/ml of each cytokine. The data presented are the mean TNF-α (FIG. 29A) and IL-1β (FIG. 29B) concentrations±SEM from serum of CD patients and healthy donors. Asterisk(s) indicate statistically significant differences, i.e., p<0.05 (*), between the 2 groups.

The results in FIG. 29 show that CD patients exhibited significant increases in TNF-α and IL-1β compared to healthy donors.

### Example 31

### Histological Characteristics of Colitis by Normal and CD Patients

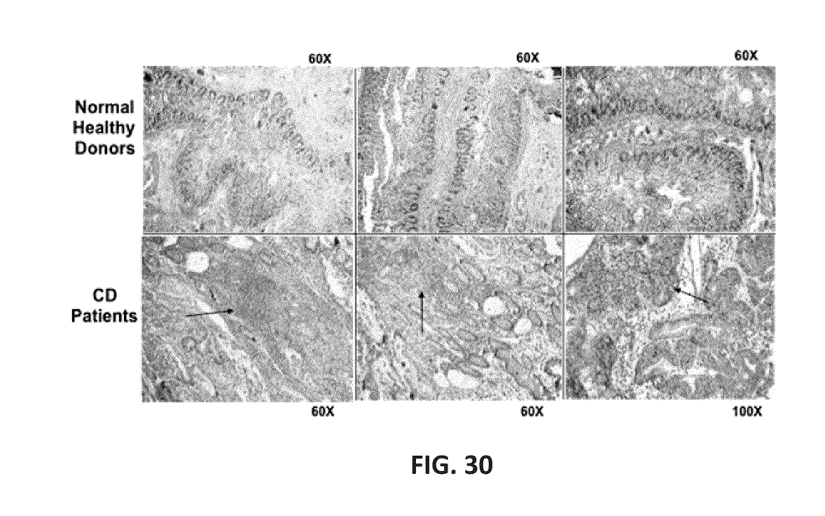

FIG. 30 shows histological characteristics of colitis in normal and CD patients with high serum CXCR3 ligand concentrations. Histopathology of colonic biopsy from normal healthy donors and CD patients were fixed, sectioned at 6 μm, and stained with hematoxylin and eosin. Sections were examined by microscopy.

FIG. 30 shows that the colon in CD patients demonstrates differences in crypt malformation, leukocyte infiltration, glandular elongation/hyperplasia, and edema between normal and CD patients.

### Example 32

### CXCL9, CXCL10, CXCL11 and TNFα Expression in Colons of CD Patients

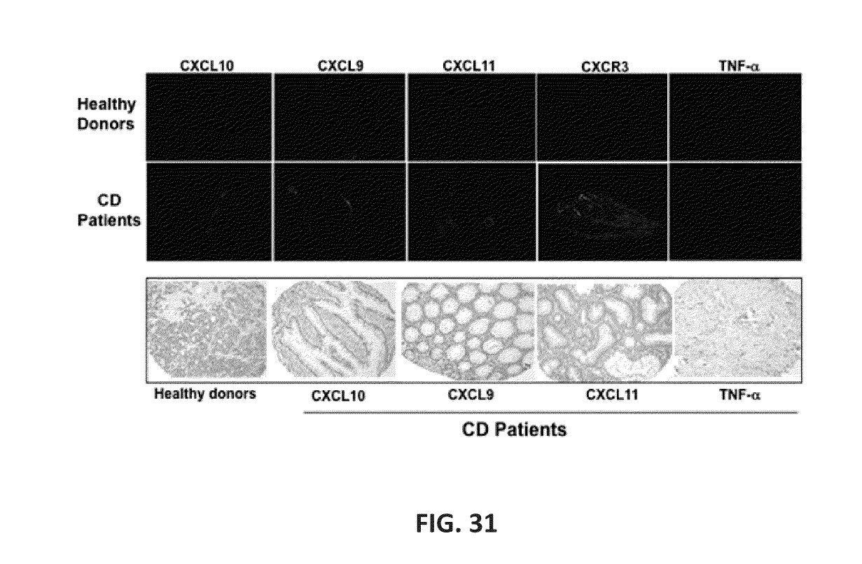

FIG. 31 shows CXCR3 ligands and TNFα expression in colons of normal and CD patients by histopathological examination. The colons from normal and CD patients were frozen, fixed, sectioned at 6 μm, and stained fluorescently for CXCL9-, CXCL10-, CXCL11- and TNFα-positive cells. Sections were examined by fluorescent con-focal microscopy.

FIG. 31 shows that the colon from a CD patient shows increased leukocyte infiltration compared with a normal control patient. These micrographs further demonstrate reduced immunoreactive staining of CXCR3 ligands and TNFα expression in colon of normal control patients.

The above description is for the purpose of teaching the person of ordinary skill in the art how to practice the present invention, and is not intended to detail all those obvious modifications and variations of it that will become apparent to the skilled worker upon reading the description. It is intended, however, that all such obvious modifications and variations be included within the scope of the present invention, which is defined by the following claims. The claims are intended to cover the components and steps in any sequence that is effective to meet the objectives there intended, unless the context specifically indicates the contrary. All the references cited in the specification are herein incorporated by reference in their entirely.

1. A method for treating an inflammatory condition in a subject, comprising:
administering to a subject in need of such treatment a therapeutically effective amount of CXCR5 antibodies antibody,
wherein said antibody is administered in a dosage range from about 10 μg/kg body weight/day to about 10 mg/kg body weight/day,
wherein said inflammatory condition is arthritis, nephritis or systemic lupus erthematosus.

2-4. (canceled)

5. The method of claim 1, wherein said antibody is administered in conjunction with a secondary anti-inflammatory agent selected from the group consisting of anti-inflammatory antibodies, short interfering RNA (siRNA), chemokine and chemokine receptor binding agents, antisense oligonucleotides, triplex forming oligonucleotides, ribozymes, external guide sequences, agent-encoding expression vectors and small molecule anti-inflammatory compounds.

6. The method of claim 5, wherein said secondary anti-inflammatory agent comprises an antibody directed against a cytokine, chemokine, or receptor thereof.

7. The method of claim 5, wherein said secondary anti-inflammatory agent comprises or encodes an siRNA that inhibits expression of CXCL9, CXCL10, CXCL11, CXCL13, CXCR5 or CXCR3.

8. The method of claim 5, wherein said secondary anti-inflammatory agent comprises a small molecule anti-inflammatory compound.

9. The method of claim 8, wherein said small molecule anti-inflammatory compound is an analgesic or non-steroidal anti-inflammatory drug (NSAID).

10. The method of claim 1, wherein said subject is diagnosed with an elevated level of CXCR5 expression.

11. The method of claim 1, wherein said anti-CXCR5 antibody binds to CXCR5 with a kd value in the range of 0.1 pM to 1 μM.

12-22. (canceled)

23. The method of claim 1, further comprising administering to said subject a therapeutically effective amount of anti-CXCL13 antibodies.

24. The method of claim 1, wherein said subject is diagnosed with an elevated level of CXCL13 expression.

25. A method for treating an inflammatory condition in a subject, comprising:
administering to a subject in need of such treatment a therapeutically effective amount of anti-CXCR5 antibody,
wherein said antibody is administered in a dosage range from about 10 μg/kg body weight/day to about 10 mg/kg body weight/day, and
wherein said inflammatory condition is nephritis.

26. The method of claim 25, wherein said anti-CXCR5 antibody is a humanized antibody.

27. The method of claim 25, wherein said anti-CXCR5 antibody is a chimeric antibody.

28. The method of claim 25, wherein said anti-CXCR5 antibody is administered directly into an inflamed tissue.

29. The method of claim 25, wherein said anti-CXCR5 antibody is administered systemically.

30. The method of claim 25, wherein said antibody is administered in conjunction with a secondary anti-inflammatory agent selected from the group consisting of anti-inflammatory antibodies, short interfering RNA (siRNA), chemokine and chemokine receptor binding agents, antisense oligonucleotides, triplex forming oligonucleotides, ribozymes, external guide sequences, agent-encoding expression vectors and small molecule anti-inflammatory compounds.

31. The method of claim 30, wherein said secondary anti-inflammatory agent comprises an antibody directed against a cytokine, chemokine, or receptor thereof.

32. The method of claim 30, wherein said secondary anti-inflammatory agent comprises or encodes an siRNA that inhibits expression of CXCL9, CXCL10, CXCL11, CXCL13, CXCR5 or CXCR3.

33. The method of claim 30, wherein said secondary anti-inflammatory agent comprises a small molecule anti-inflammatory compound.

34. The method of claim 33, wherein said small molecule anti-inflammatory compound is an analgesic or non-steroidal anti-inflammatory drug (NSAID).
